(19) United States
(12) Patent Application Publication
(10) Pub. No.: US 2003/0008309 A1
(43) Pub. Date: Jan. 9, 2003

(54) Microarrays and methods for evaluating activity of compounds having estrogen-like activity

(75) Inventors: Ryoiti Kiyama, Ibaraki (JP); Shinobu Oguchi, Tokyo (JP)

Correspondence Address: FOLEY AND LARDNER, SUITE 500, 3000 K STREET NW, WASHINGTON, DC, 20007, US

(73) Assignee: National Institute of Advanced Industrial Science and Technology

(21) Appl. No.: 10/120,780

(22) Filed: Apr. 12, 2002

Related U.S. Application Data
(63) Continuation-in-part of application No. 09/975,316, filed on Oct. 12, 2001, now abandoned.
(60) Provisional application No. 60/300,835, filed on Jun. 27, 2001.

(30) Foreign Application Priority Data
Oct. 13, 2000 (JP) 2000-313403
Oct. 15, 2001 (JP) 2001-317152

(51) Int. Cl. C12Q 1/68; C12M 1/34

(52) U.S. Cl. 435/6; 435/287.2

(57) ABSTRACT

In order to detect the effects of environmental hormones, the effect of chemical substances having estrogen-like activity are detected and evaluated.

This invention is characterized in that DNA fragments containing portions or wholes of genes and/or ESTs (Expressed Sequence Tags) whose expression is affected by chemical substances having estrogen-like activity are immobilized on the basal plate of the microarray.

### FIELD OF THE INVENTION

This invention relates to a microarray comprising DNA probes immobilized on the basal plate thereof and a method for evaluating activity of compounds having estrogen-like activity that uses this microarray.

### BACKGROUND OF THE INVENTION

So far, the so-called environmental hormones are known to be chemical substances such as those that have estrogen-like activity as natural estrogens, 17β-estradiol for example, do. These environmental hormones are endocrine disrupting chemicals towards the human body and have greatly deleterious effects on each of the tissues within the human body. Therefore, precise evaluation of the activity of environmental hormones, together with detection of environmental hormones, in each of the tissues of the human body is desired. Although detection of environmental hormones by sampling each of the tissues of the human body has been possible, detection of the activity of environmental hormones in each of the tissues has not been possible.

Microarrays that are used for monitoring gene expression, determination and variant analysis of nucleotide sequence, and such are known. A microarray is also called a DNA chip and comprises a basal plate made of slide glass, silicone, or the like, and DNA fragments immobilized as an array on this basal plate. With this microarray, DNAs contained in a sample can be detected by hybridizing them with the DNA fragments immobilized on the basal plate. Since the DNA within the sample is radiolabeled or fluorescently labeled, detection with radio imaging scanner, fluorescence imaging scanner, or the like is possible.

In this way, microarrays enable quantitatively detecting DNA contained within a sample and monitoring gene expression and the like. However, methods for evaluating the detection and the effect of the above-mentioned estrogen using microarrays are not established.

### SUMMARY OF THE INVENTION

This invention has been made in view of the current state of affairs as mentioned above, and an objective of this invention is to provide microarrays capable of detecting and evaluating the effect of chemical substances having estrogen-like activity and methods for evaluating activity of compounds having estrogen-like activity, for the purpose of detecting the effect of environmental hormones.

The microarray of this invention, which has accomplished the above-mentioned objective, is characterized in that DNA fragments containing entire genes, portions thereof, ESTs (Expressed Sequence Tags) thereof, or portions of the ESTs, wherein the expression of the DNA fragments is affected by chemical substances having estrogen-like activity, are immobilized on the basal plate of the microarray.

In this invention, it is preferable that the DNA fragments are selected from the group consisting of genes, gene portions, ESTs, or portions of the ESTs whose expression level changes in breast cancer-derived MCF-7 cells in the presence of estrogen.

Furthermore, it is preferable that the above-mentioned DNA fragments belong to any one of groups (1), (2), and (3) below:

group (1): genes, gene portions, ESTs, or portions of the ESTs whose expression level increases due to the presence of chemical substances having estrogen-like activity,

group (2): genes, gene portions, ESTs, or portions of the ESTs whose expression level decreases due to the presence of chemical substances having estrogen-like activity, and

group (3): genes, gene portions, ESTs, or portions oftheESTs whose change in expression level arising due to the presence of a chemical substance having estrogen-like activity is smaller than that of groups (1) and (2) and whose expression level arising due to the presence of the chemical substance having estrogen-like activity is the same as or not less than that of a gene, gene portion, EST, or portion of the EST that is constitutively expressed regardless of the presence or absence of the chemical substance having estrogen-like activity.

Also, it is preferable that, in the microarray, more than one DNA fragment(s) selected from the group consisting of genes, gene portions, ESTs, or portions of the ESTs belonging to the group (1) above, genes, gene portions, ESTs, or portions of the ESTs belonging to the group (2) above, and genes, gene portions, ESTs, or portions of the ESTs belonging to the group (3) above are further immobilized on the basal plate.

Furthermore, it is preferable that, in the microarray of this invention, additional DNA fragments comprising genes, gene portions, ESTs, or portions of the ESTs that are constitutively expressed regardless of the presence or absence of chemical substances having estrogen-like activity are further immobilized on the basal plate of the microarray.

On the other hand, in the microarray of this invention, it is preferable that the above-mentioned DNA fragments belong to any one of groups (4), (5), (6), (7), and (8) below:

group (4): genes, gene portions, ESTs, or portions of the ESTs related to cancer,

group (5): genes, gene portions, ESTs, or portions of the ESTs involved in cell proliferation and/or division,

group (6): genes, gene portions, ESTs, or portions of the ESTs encoding polypeptides functioning as receptors,

group (7): genes, gene portions, ESTs, or portions of the ESTs involved in transcription reaction, and

group (8): genes, gene portions, ESTs, or portions of the ESTs involved in cell structure.

Here, it is preferable that more than one DNA fragment(s) selected from the group consisting of genes, gene portions, ESTs, or portions of the ESTs belonging to the group (4) above, genes, gene portions, ESTs, or portions of the ESTs belonging to the group (5) above, genes, gene portions, ESTs, or portions of the ESTs belonging to the group (6) above, genes, gene portions, ESTs, or portions of the ESTs belonging to the group (7) above, and genes, gene portions, ESTs, or portions of the ESTs belonging to the group (8) above are further immobilized on the basal plate.

Also, it is preferable that, in the micro array of this invention, the above-mentioned DNA fragments belong to any one of groups (9) (10), and (11) below:

group (9): genes, gene portions, ESTs, or portions of the ESTs functioning in tissues on which estrogen acts,

group (10): genes, gene portions, ESTs, or portions of the ESTs characteristic of epithelial cells, and

group (11): genes, gene portions, ESTs, or portions of the ESTs functioning in nerve cells.

Here, it is preferable that more than one DNA fragment(s) selected from the group consisting of genes, gene portions, ESTs, or portions of the ESTs belonging to the group (9) above, genes, gene portions, ESTs, or portions of the ESTs belonging to the group (10) above, and genes, gene portions, ESTs, or portions of the ESTs belonging to the group (11) above are further immobilized on the basal plate.

Furthermore, it is preferable that, in the microarray of this invention, the above-mentioned DNA fragments belong to group (12) or (13) below:

group (12) genes, gene portions, ESTs, or portions oftheESTs whose expression level increases owing to estrogen in cells resistant to antagonists of estrogen, and

group (13) genes, gene portions, ESTs, or portions of the ESTs whose expression level decreases owing to estrogen in cells resistant to antagonists of estrogen.

Here, it is preferable that more than one DNA fragment(s) selected from the group consisting of genes, gene portions, ESTs, or portions of the ESTs belonging to the group (12) above and genes, gene portions, ESTs, or portions of the ESTs belonging to the group (13) above are further immobilized on the basal plate.

On the other hand, the method of this invention for evaluating the estrogen-like activity of a compound comprises utilizing the microarray of any one of claims 1 to 11.

Furthermore, this invention relates to a method for predicting the effect on a cell of a compound having estrogen-like activity, the method comprising the steps of:

(a) preparing a nucleic acid sample from a cell to be evaluated,

(b) contacting the nucleic acid sample with the microarray of any one of claims 1 to 11,

(c) detecting a nucleic acid hybridizing with the microarray, and

(d) comparing a result detected in step (c) with a result detected using a nucleic acid sample prepared from a control cell.

Here, it is preferable that the control cell is an MCF-7 cell treated with 17β-estradiol.

### BRIEF DESCRIPTION OF THE DRAWINGS

FIG. 1 is a diagram indicating the result of evaluating estrogen-like activity using the microarray after treating MCF-7 cells with 10 nM dioxin and then obtaining the mRNA.

In the experiments of FIGS. 1 to 13, mRNA derived from cells treated with chemical substances was labeled using Cy3, and as a control, mRNA obtained from untreated cells was labeled using Cy5. Each of the vertical and horizontal axes indicates the fluorescence intensity of either label using convenient units. MCF-7 cells and RL95-2 cells are breast cancer cells and endometrial cancer cells, respectively.

FIG. 2 is a diagram indicating the result of evaluating estrogen-like activity using the microarray after treating MCF-7 cells with 1 nM dioxin and then obtaining the mRNA.

FIG. 3 is a diagram indicating the result of evaluating estrogen-like activity using the microarray after treating MCF-7 cells with 0.1 nM dioxin and then obtaining the mRNA.

FIG. 4 is a diagram indicating the result of evaluating estrogen-like activity using the microarray after treating RL95-2 cells with 10 nM dioxin and then obtaining the mRNA.

FIG. 5 is a diagram indicating the result of evaluating estrogen-like activity using the microarray after treating RL95-2 cells with 1 nM dioxin and then obtaining the mRNA.

FIG. 6 is a diagram indicating the result of evaluating estrogen-like activity using the microarray after treating RL95-2 cells with 0.1 nM dioxin and then obtaining the mRNA.

FIG. 7 is a diagram indicating the result of evaluating estrogen-like activity using the microarray after treating MCF-7 cells with 10 nM estrone and then obtaining the mRNA.

FIG. 8 is a diagram indicating the result of evaluating estrogen-like activity using the microarray after treating MCF-7 cells with 1 nM estriol and then obtaining the mRNA.

FIG. 9 is a diagram indicating the result of evaluating estrogen-like activity using the microarray after treating MCF-7 cells with 10 nM genistein and then obtaining the mRNA.

FIG. 10 is a diagram indicating the result of evaluating estrogen-like activity using the microarray after treating MCF-7 cells with 10 nM nonylphenol and then obtaining the mRNA.

FIG. 11 is a diagram indicating the result of evaluating estrogen-like activity using the microarray after treating MCF-7 cells with 10 nM bisphenol A and then obtaining the mRNA.

FIG. 12 is a diagram indicating the result of evaluating estrogen-like activity using the microarray after treating MCF-7 cells with 10 nM diethylstilbestrol and then obtaining the mRNA.

FIG. 13 is a diagram indicating the result of evaluating estrogen-like activity as control data using the microarray after treating MCF-7 cells with 10 nM 17 β-estradiol and then obtaining the mRNA.

### DETAILED DESCRIPTION OF THE INVENTION

The microarrays and the methods of this invention for evaluating activity of compounds having estrogen-like activity are described in detail below.

The microarray to which this invention is applied has multiple DNA fragments immobilized on the basal plate of the microarray. Here, DNA fragments contain a portion or the whole of genes and/or ESTs whose expression is affected by chemical substances having estrogen-like activity. Specifically, examples of the genes and ESTs include those indicating responsiveness towards 17β-estradiol in a breast cancer-derived MCF-7 cell line or MCF-7 cell line-derived cell line. That is, in a breast cancer-derived MCF-7 cell line or MCF-7 cell line-derived cell line, it is possible to use DNA fragments containing a portion or the whole of the genes or ESTs whose expression level changes in response to the presence or absence of 17β-estradiol.

Herein, “expression level changes in response to the presence or absence of 17β-estradiol” means that either the increase in the amount of mRNA is twofold or more when 17β-estradiol is present, or the decrease in the amount of mRNA is ½ or lower when 17β-estradiol is present.

Examples of genes or ESTs indicating responsiveness towards 17β-estradiol in a breast cancer-derived MCF-7 cell line or MCF-7 cell line-derived cell line include

group (1): those whose expression level increases owing to 17β-estradiol,

group (2): those whose expression level decreases owing to 17β-estradiol, and

group (3): those whose change in expression level arising due to the presence of a chemical substance having estrogen-like activity is smaller than that of (1) and (2) above and whose expression level arising due to the presence of the chemical substance having estrogen-like activity is the same as or not less than that of a gene, gene portion, EST, or portion of the EST that is constitutively expressed regardless of the presence or absence of the chemical substance having estrogen-like activity.

Herein, the group (1) means those whose expression level increases to threefold or more owing to the presence of 17β-estradiol, and the group (2) means those whose expression level decreases to ⅓ or lower owing to the presence of 17β-estradiol. In addition, the group (3) means those whose expression level increases to more than twofold and less than threefold or decreases to more than ⅓ and less than ½ owing to the presence of 17β-estradiol and whose expression level is the same as or not less than the expression level of keratin 6B, which is one of the genes used as a negative control.

More specifically, examples of genes or ESTs of the group (1) include

activating transcription factor 3 {N39944};

aldo-keto reductase family 1, member C4 {T73188};

amphiregulin {M30704};

asparagine synthetase {L35946};

B-factor, properdin {X72875};

cyclin A1 {U66838};

DKFZP566G223 protein {AI261366};

early growth response 3 {X63741};

ESTs {AA587912};

ESTs {AI185199};

ESTs {U82984};

ESTs {X62585};

ESTs {N35555};

ESTs {AA975298};

ESTs {AA682502};

ESTs {AA629308};

exportin, tRNA {AF039022};

FOS-like antigen 2 {AI088306};

FOS-like antigen 2 {X16706};

glutamic-oxaloacetic transaminase 1, soluble {M37400};

glutamine-fructose-6-phosphate transaminase 1 {M90516};

glycyl-tRNA synthetase {D30658};

heat shock 70-kD protein 5 {AI878886};

Homo sapiens ccr2b (ccr2), ccr2a (ccr2), ccr5 (ccr5) and ccr6 (ccr6) genes {AF012281};

Homo sapiens integrin-linked kinase (ILK), 1 {XM_{—}006424};

Homo sapiens integrin-linked kinase (ILK), 2 {XM-006424};

Homo sapiens methylene tetrahydrofolate dehydrogenase {XM_{—}002234};

Homo sapiens partial mRNA for A-type potassium channel modulatory protein 2 {AJ276317};

Homo sapiens similar to chemokine receptor 2 {XM_{—}002923};

Homo sapiens solute carrier family 26, member 3 {L02785};

Homo sapiens transcription elongation factor A {NM-006756};

hyaluronan-mediated motility receptor {U29343};

insulin-like growth factor-binding protein 4 {M62403};

interferon stimulated gene {X89773};

KIAA1051 protein {AB028974};

matrix Gla protein {AA484893};

methylene tetrahydrofolate dehydrogenase {X16396};

microtubule-associated protein 1B {L06237};

neuropeptide Y receptor Y1 {NM-00090};

nuclear receptor interacting protein 1 {X84373};

phorbol-12-myristate-13-acetate-induced protein 1 {D90070};

phosphoenolpyruvate carboxykinase 2 {X92720};

phosphoinositide-3-kinase, class 3 {Z46973};

prostate differentiation factor {AA216685};

retinoblastoma-binding protein 8 {U72066};

ribosomal protein S6 kinase, 90-kD, polypeptide 3 {U08316};

S100 calcium-binding protein P {AI151190};

solute carrier family 7, member 5 {M80244};

stanniocalcin 2 {AB012664};

TGFB inducible early growth response {AF050110};

transcobalamin I {J05068};

trefoil factor 1 {X05030};

tumor protein D52-like 1 {U44427};

tumor rejection antigen (gp96) 1 {X15187};

unc-51-like kinase 1 {AB018265}; and v-jun avian sarcoma virus 17 oncogene homolog {AI598150}. GenBank accession numbers are indicated in parentheses.

Examples of genes or ESTs of the group (2) include

absent in melanoma 1 {U83115};

bassoon {Y18448};

cadherin 18 {U59325};

catenin, delta 2 {XM-011351};

EGF-containing fibulin-like extracellular matrix protein 1 {U03877};

enolase 3 {X56832};

Estrogen receptor, alpha {AY033393};

fucosyltransferase 8 {Y17977};

heat shock 70-kD protein 1 {M59828};

Homo sapiens CDC14 homolog B {XM-011585};

Homo sapiens paired basic amino acid cleaving system 4 {NM-002570};

Homo sapiens quiescin Q6 {NM-002826};

Homo sapinens mRNA;cDNA DKFZp434J065 {AL137638};

Human insulin-like growth factor binding protein 5 {L27560};

Human mRNA for KIAA0018 gene {D13643};

insulin-like growth factor binding protein 5 {AA374325};

KIAA0307 gene product {AB002305};

paired basic amino acid cleaving system 4 {D87993};

quiescin Q6 {U97276};

Rho GDP dissociation inhibitor (GDI) alpha {X69550};

selenium binding protein 1 {U29091}; and

solute carrier family 12, member 2 {U30246}.

Examples of genes or ESTs of the group (3) include

aconitase 2, mitochondrial {U87939};

alpha-1-antichymotrypsin {X00947};

argininosuccinate synthetase {AI660571};

calpain, small polypeptide {AC002984};

carnitine palmitoyltransferase I {L39211};

cathepsin D {M63138};

CDC6 homolog {AF022109};

CDP-diacylglycerol-inositol 3-phosphatidyltransferase {AF014807};

chromobox homolog 1 {AL046741};

corneodesmosin {L20815};

enolase 2 {X51956};

ESTs {AI949781};

ESTs {AI188401};

ESTs {AI492976};

ESTs {AI767533};

ESTs {AI332415};

eukaryotic translation initiation factor 3, subunit 9 {U78525};

ferritin, heavy polypeptide 1 {AA102267};

fructose-bisphosphatase 1 {M19922};

general transcription factor II, i, pseudogene 1 {AI700706};

H3 histone, family 3B {Z48950};

histone deacetylase 6 {AJ011972};

Homo sapiens, adipose specific 2 {BC004471};

Homo sapiens BAC clone CTB-163k11 from 7q31 {AC005192};

Homo sapiens cDNA FLJ12748fis {AK022810};

Homo sapiens chromosome 17, Neurofibromatosis 1 locus {AC004526};

Homo sapiens clone 23783 mRNA sequence {AF054996};

Homo sapiens DNA from chromosome 19, cosmid F21856 {AC004030};

Homo sapiens interleukin 2 receptor, beta {XM-009962};

Homo sapiens mRNA for KIAA0226 protein {D86979};

Homo sapiens mRNA for NEFA protein {X76732};

Homo sapiens ryanodine receptor 2 {XM-001778};

Homo sapiens vascular endothelial growth factor {AF024710};

HS1 binding protein {N28312};

Human breast cancer cytosolic NADP(+)-dependent malic enzyme {U43944};

Human clone 23855 mRNA {U79302};

Human low-Mr GTP-binding protein {U59878};

Human meningioma-expressed antigen II {U73682};

Human mRNA for KIAA0373 gene {AB002371};

Human threonyl-tRNA synthetase {M63180};

Human transcripton factor ERF-1 {U85658};

inhibitor of DNA binding 4, dominant negative helix-loop-helix protein {AL022726};

isoleucine-tRNA synthetase {NM-012096};

keratin 8 {X74929};

KIAA0058 gene product {AL036958};

KIAA0123 protein {D21064};

KIAA0196 gene product {D83780};

KIAA0551 protein {AB011123};

KIAA0587 gene product {AB011159};

mal, T-cell differentiation protein {X76220};

mannosidase, alpha, class 1A, member 1 {X74837};

membrane component, chromosome 1, surface marker 1 {X13425};

MYC promoter-binding protein 1 {AA482422};

poliovirus receptor {X64116};

protein geranylgeranyltransferase type I, beta subunit {AA481712};

protein kinase C substrate 80K-H {J03075};

protein kinase C, delta {D10495};

protein tyrosine phosphatase, non-receptor type 18 {X79568};

ras homolog gene family, member C {L25081};

reticulocalbin 1, EF-hand calcium binding domain {D42073};

serine hydroxymethyltransferase 2 {U23143};

SH3-domain binding protein 5 {AB005047};

solute carrier family 1, member 5 {AF105826};

sorbitol dehydrogenase {U07361};

stearoyl-CoA desaturase {AA143530};

synaptogyrin 2 {AJ002308};

TATA box binding protein (TBP)-associated factor {U21858};

tyrosyl-tRNA synthetase {U89436};

U5 snRNP-specific protein {D21163}; and UDP-Gal:betaGlcNAc beta 1,4-galactosyltransferase, polypeptide 1 {X13223}.

Furthermore, in this invention, the genes and/or ESTs to be immobilized on the basal plate are not limited to those belonging to the above-mentioned groups (1), (2), and (3), and any genes and/or any ESTs may be used as long as their expression is affected by chemical substances having estrogen-like activity. The term “chemical substances having estrogen-like activity” used herein means a chemical substance that affects female reproductive organs and such similarly to a human sex hormone, estrogen (a general term for hormones that are steroidal chemical substances secreted from ovarian follicles, placenta, and such and that induce the development of female reproductive organs such as follicles and mammary glands or other organs (such as brain)). Although the functional mechanism of estrogen itself is not yet completely understood, estrogen is thought to affect the expression of other genes by binding to estrogen receptors. Therefore, estrogen is thought to affect tissues such as brain, liver, muscle, bone cells, stomach, and so on where estrogen receptor gene is expressed.

Examples of chemical substances having estrogen-like activity include natural or synthesized female hormones such as 17β-estradiol, estrone, estriol, diethylstilbestrol, and such. In addition, examples of chemical substances having estrogen-like activity include plant estrogens such as coumestrol and daidzein. Furthermore, examples of chemical substances having estrogen-like activity include agricultural chemicals such as DDT, aldrin, and heptachlor. Furthermore, examples of chemical substances having estrogen-like activity include industrial raw materials, materials, products, and by-products such as dioxin, PCB, nonyphenol, bisphenol A, phthalic acid esters, and tributyltin compounds.

The DNA fragment to be immobilized on the basal plate may contain the whole or a portion of the genes and/or the ESTs described above. The term “a portion of a gene and/or an EST” used herein means a portion of the gene and/or the EST described above and a nucleotide sequence equivalent to at least 300 to 500 base pairs.

In addition, it is preferable that, in a microarray, genes and/or ESTs constitutively expressing regardless of the presence or absence of chemical substances having estrogen-like activity (hereinafter, referred to as negative control genes and the like) are immobilized on the basal plates. For the negative control genes and the like, it is preferable to select several of those that have differences in their expression levels. Examples of genes that constitutively express regardless of the presence of absence of chemical substances having estrogen-like activity and that have differences in their expression levels include

actin, alpha 1 {BC003575};

actin, beta {X00351};

general transcription factor IIH {AI239815};

glutamate-cysteine ligase {L35546};

H. sapiens tunp mRNA for transformation {X72727};

Homo sapiens cDNA FLJ12748fis, clone NT2RP2001137 {AK0022810};

Homo sapiens interleukin 6 signal transducer {NM002184};

Homo sapiens protein kinase C delta {XM003106};

Homo sapiens protein kinase related to S.cerevisiae STE20 {XM009395};

Homo sapiens putative transmembrane protein {XM005791};

Homo sapiens STAT-induced STAT inhibitor-2 {AF037989};

Homo sapiens, short-chain deghydrogenase/reductase 1 {BC002730};

Homo sapiens, TLS-associated serine-arginine protein 2 {BC005039};

Human DNA sequence from PAC 204E5 on chromosome 12 {Z98941};

Human GOS2 gene {M72855};

Human GT mitochondrial solute carrier protein {M31659};

Human mRNA for glucose-6-phosphate dehydrogenase (G6PD). {X03673};

Human mRNA for KIAA0349 gene {AB002347};

Human pre-B cell stimulating factor homologue (SDF1b) {L36033};

keratin 6B {L42611};

Macaca fascicularis brain cDNA, clone: QnpA-21739 {AB050427};

mRNA from KIAA 1151 protein {AB032977};

nucleophosmin {AA173870};

ribosomal protein L35 {AI815757};

solute carrier family 29 (nucleoside transporters), member 2 {AI342303};

tumor necrosis factor receptor superfamily, member 7 {AA994925}; and zinc finger protein 147 {D21205}.

The expression level of the genes and/or ESTs of the groups (1) (2), and (3) can be corrected by immobilizing negative control genes and such on the basal plate. That is, by correcting so that the expression level of the negative control genes and such may be always constant, the expression level of the genes and/or ESTs of the groups (1), (2), and (3) can be corrected. Thus, the changes in the expression level of genes and/or ESTs of the groups (1), (2), and (3) can be detected with certainty.

The expression level of genes and/or ESTs indicated in the groups (1), (2), and (3) may be corrected with accuracy by choosing several of negative control genes and such that have different expression levels. That is, when the expression level of a particular gene and/or EST is to be corrected, correction can be carried out with accuracy using negative control genes and such that indicate expression levels close to the expression levels of the genes and/or ESTs in question.

In addition, in the microarray, genes and/or ESTs whose expression shows no remarkable increase or decrease in 17β-estradiol-treated MCF-7 cells and whose relation to estrogen is known or predicted in the effect of estrogen on cells may be used as positive controls (hereinafter, these are referred to as positive control genes and such). That is, a gene such as estrogen receptor beta gene that functions as an estrogen receptor in tissues such as brain, aromatase gene or 17 beta-HSD gene that indicates elevated expression due to estrogen or compounds having estrogen-like activity, or CYP1A1 gene known as a marker for dioxin can be used for performing an evaluation with higher accuracy.

Examples of positive control genes and such include

17 beta-HSD, type 2 {AF411015};

A1B1 {AF012108};

AhR {XM040803};

aromatase {AF397171};

cyclin D {AF318615};

CYP1A1 {AH007293};

erbB2 {M86910};

Estrogen receptor, beta {NM001437};

Estrogen sulfatase {L25275};

H.sapiens SCA1 mRNA for ataxin {X79203};

Homo sapiens cDNA FLJ12048fis, clone HEMBB1001990 {AK022110};

Homo sapiens mRNA full length insert cDNA clone EUROIMAGE781354 {M60922};

Homo sapiens ribosormal protein L31 {NM000993};

Homo sapiens transforming growth factor, alpha {XM002732};

Homo sapiens UDP-Gal: betaGlcNAc beta 1,4-galactocyltransferase, polypeptide 1 {NM001497};

Human 150-kDa oxgen-regulated protein ORP150 {U65785};

Human autotaxin-t {L46720};

Human chromosome 14 DNA sequence BAC R-406H23oq library {AL118556};

Human inositol 1,3,4-trisphosphate 5/6-kinase {U51335};

Human intercrine-alpha {U19495};

Human mRNA amd promoter DNA for progesteron receptor {X51730};

Human mRNA for KIAA0008 {D13633};

Human mRNA for KIAA0062 gene {D31887};

Human mRNA for KIAA0101 gene {D14656};

IGFR {JQ1035};

KIAA0172 {D79994};

progesterone receptor {XP006190}; and SRC-1 {U40396}.

When the DNA fragments and negative control genes are immobilized on the basal plate, a conventionally known technique can be used. For example, the surface of the basal plate can be treated with polycations such as polylysines to electrostatically bind the DNA fragments through their charges on the surface of the basal plate, and techniques to covalently bind the 5′-end of the DNA fragments to the basal plate may be used. Also, a basal plate that has linkers on its surface can be produced, and functional groups that can form covalent bonds with the linkers can be introduced at the end of the DNA fragments. Then, by forming a covalent bond between the linker and the functional group, the DNA fragments and such can be immobilized.

The expression levels of the genes and/or ESTs indicating responsiveness towards estrogen in the cell to be evaluated can be measured by using a micro array composed as described above. Measuring the expression level of genes and/or ESTs in the cell to be evaluated enables evaluation of the effects of chemical substances having estrogen-like activity, such as environmental hormones, etc.

In this invention, there are no particular limitations on cells to be evaluated. Cells obtained from human tissues such as mammary glands, uterus, liver, brain, stomach, and such may be used as cells to be evaluated. To evaluate the effect of environmental hormones and such in the cells to be evaluated, first, total mRNA is extracted from the cells. When mRNA is extracted, a conventionally known technique may be used, and a commercially available mRNA purification kit (for example, MACS ; Miltenyi Biotec) may be used.

Next, with the extracted mRNA as a template, cDNAs are produced using a reverse transcriptase (for example, SuperScript Reverse Transcriptase ; GibcoBRL) and labeled dNTP (for example, Cy3-dUTP and Cy5-dUTP ; Amersham Pharmacia Biotech), and a cDNA sample that reflects the amount of genes expressed within the cells to be evaluated is prepared. This causes labeled cDNA to be included in the cDNA sample. Here, either fluorescent label or radiolabel may be used as a label.

The cDNA sample prepared in this manner is applied to the above-mentioned microarray in its single stranded denatured form, and cDNAs included in the cDNA sample are hybridized with the genes and/or ESTs immobilized on the basal plate. Here, hybridization with cDNA can be accomplished, for example, by incubating at 65° C. for 10 to 20 hours.

Then, cDNAs that hybridized with the DNA fragments on the microarray are detected. In cases where the hybridized cDNAs are fluorescently labeled, the fluorescence is detected with, for example, a fluorescence laser microscope and a CCD camera, and the fluorescence intensity is analyzed with a computer. Similarly, in cases where the hybridized cDNAs are radiolabeled, detection can be carried out using an RI image scanner and such, and the intensity of the radiation can be analyzed with a computer.

The effect of chemical substances having estrogen-like activity on the cells to be evaluated can be determined using a microarray by detecting the expression level of the above-mentioned genes and/or ESTs in the cells to be evaluated. In other words, using the above-mentioned microarray to measure the expression of genes and ESTs in these cells, the effects of environmental hormones and such may be evaluated.

This evaluation uses an evaluation value that represents the sum of expression levels of genes and/or ESTs contained in the DNA fragments immobilized on the basal plate, as a relative value to the sum of expression levels of the genes and/or ESTs in MCF-7 cells in the presence of 10 nM 17β-estradiol. That is, when the sum of expression levels of the genes and/or ESTs in MCF-7 cells in the presence of 10 nM 17β-estradiol is taken to be equal to the sum of the total expression level of the expression levels of the genes and/or ESTs in MCF-7 cells in the presence of a test chemical substance, the evaluation value is calculated as the sum of the absolute values of the ratios of expression level of each gene or EST in the presence of 10 nM 17β-estradiol to expression level of the gene or EST in the presence of the test chemical substance.

Calculation procedure for the evaluation value is described below.

The expression level of the n-th gene or EST, En (n=1 to 203) can be defined as follows:

En=(Cy3/Cy5 corrected value)

where Cy3/Cy5 corrected value=(Cy3/Cy5 value)/(average value of Cy3/Cy5 value for negative control genes and the like). That is, Cy3/Cy5 corrected value is a Cy3/Cy5 value of each gene that has been corrected with the expression level of the negative control genes and the like. A total of 27 genes are selected as the negative control genes and the like, but this may be changed depending on expression conditions and such.

Next, to compare the expression level of genes and ESTs in treatment with various chemical substances, the expression level is represented as follows using corrected value, t:

BDEn=t(En)

where the expression level after correction is represented by BDEn (n=1 to 203). The standard of t value is the expression level when 17β-estradiol is used, E(Estrogen)n. That is, if the expression level when chemical substance A is used is represented by E (A) n, the t value is calculated according to

t=Σ[E(Estrogen)n]/Σ[E(A)n]

where, for 17β-estradiol,

t=1,

and therefore, it follows that

BDEn=En.

When estrogen-like activity is evaluated, the expression level after correction when 17β-estradiol is used is represented by BDE(Estrogen)n and the expression level of gene n after correction when chemical substance A is used is represented by BDE(A)n. Then, the absolute value of the difference between BDE (A)n and BDE(Estrogen)n is calculated, and their sum (n=1 to 203) is taken as the evaluation value.

(evaluation value)=Σ[|log{BDE(A)n/BDE(Estrogen)n}|]

As a result, it can be evaluated that the lower the evaluation value is, the higher the evaluated estrogen-like activity is.

When such an evaluation is performed, in an example, the results of completely independent tests with the same estrogen treatment show

(evaluation value)=16.81,

and, for example, in the case of 10 nM dioxin treatment,

(evaluation value)=46.19,

indicating a significantly higher value. This shows that the lower the evaluation value is, the more similar the effect is to estrogen (17β-estradiol) treatment.

Alternatively, the evaluation value can be calculated by considering a weighting factor based on the characteristics of genes and ESTs whose expression levels are measured with DNA fragments immobilized on the basal plate. That is, a weighting factor Kn(n=1 to 203) can be predetermined in advance for each of the 203 types of genes and ESTs including the negative and positive control genes and the like, and the evaluation value can be calculated as follows:

(evaluation value)=[Σ[Kn|log{BDE(A)n/BDE(Estrogen)n}|]]/Σ(Kn)

where n=1 to 203.

Calculation of the evaluation value in this manner, considering the weighting factors for the genes and ESTs, makes the resulting evaluation value clearly reflect the effect of chemical substances having estrogen-like activity in the cells to be evaluated. For the negative control genes and such, the weighting factor is taken to be zero in the calculation.

Here, weighting factor Kn is not set in a restrictive manner according to genes and/or ESTs but rather, it is preferable that this value is changed, considering the current state of the evaluation results, in order to obtain a more accurate evaluation result. Especially, the value of weighting factor Kn is preferably set to various values depending on the tissue from which the cells to be evaluated are derived, and on the compound having estrogen-like activity. Accordingly, this evaluation method enables a highly accurate evaluation tailored to an objective.

In addition, by using the cells obtained from human tissues such as mammary glands, uterus, liver, brain, and stomach as evaluation subjects, this method can distinguish the effect of chemical substances having estrogen-like activity on these tissues. That is, this microarray enables evaluation of the effect of chemical substances having estrogen-like activity on the tissues.

In addition, this microarray not only allows evaluation of the effect of chemical substances having estrogen-like activity on the tissues as described above, but it can also elucidate the estrogen activity of known or unknown chemical substances. That is, by using this microarray for chemical substances whose effects on cells are unknown, one can evaluate whether or not these substances have estrogen-like activity.

Furthermore, this microarray allows distinguishment of whether an activity is enhanced or inhibited when a chemical substance having estrogen-like activity coexists with another chemical substance.

The microarray of this invention not only evaluates the effect of chemical substances having estrogen-like activity on cells to be evaluated, as described above, but can also indicate the details of this effect. Especially, in the above-mentioned microarray, a total of 176 kinds of genes and ESTs comprising genes and ESTs of the groups (1), (2), and (3) and positive control genes and such can be categorized into

group (4): genes and/or ESTs related to cancer,

group (5): genes and/or ESTs involved in cell proliferation and/or division,

group (6): genes and/or ESTs encoding polypeptides functioning as receptors,

group (7): genes and/or ESTs involved in transcription reaction, and

group (8): genes and/or ESTs involved in cell structure.

Specifically, examples of genes and/or ESTs of the group (4) related to cancer (expression genes or cancer suppressor genes) include

trefoil factor 1 {X05030};

tumor protein D52-like 1 {U44427};

phorbol-12-myristate-13-acetate-induced protein 1 {D90070};

down-regulated in adenoma {L02785};

retinoblastoma-binding protein 8 {U72066};

tumor rejection antigen (gp96) 1 {X15187};

v-jun avian sarcoma virus 17 oncogene homolog {AI598150};

pituitary tumor-transforming 1 {AA430241};

ras homolog gene family, member C {L25081};

MYC promoter-binding protein 1 {AA482422}; and

absent in melanoma 1 {U83115}.

Examples of genes and/or ESTs the group (5) related to cell

proliferation and/or division include

early growth response 3 {X63741};

amphiregulin {M30704};

insulin-like growth factor-binding protein 4 {M62403};

cyclin A1 {U66838};

TGFB inducible early growth response {AF050110};

CDC6 homolog {AF022109};

catenin, delta 2 {U96136};

cadherin 18 {U59325};

EGF-containing fibulin-like extracellular matrix protein 1 {U03877};

insulin-like growth factor binding protein 5 {AA374325};

Human insulin-like growth factor binding protein5 (IGFBP5) {L27560};

cyclin A1 {U66838}; and

ribosomal protein S6 kinase, 90-kD, polypeptide 3 {U08316}.

Examples of genes and/or ESTs of the group (6) encoding polypeptides functioning as receptors include

hyaluronan-mediated motility receptor (RHAMM) {U29343};

neuropeptide Y receptor Y1 {M84755};

nuclear receptor interacting protein 1 {X84373};

poliovirus receptor {X64116}; and

estrogen receptor 1 {X03635}.

Examples of genes and/or ESTs of the group (7) involved in transcription reaction include

CCAAT/enhancer binding protein (C/EBP), beta {AA557306};

FOS-like antigen 2 {AI088306};

prostate differentiation factor {AA216685};

activating transcription factor 3 {N39944};

FOS-like antigen 2 {X16706};

H3 histone, family 3B (H3.3B) {Z48950};

TATA box binding protein (TBP)-associated factor, RNA polymerase II, G {U21858 };

histone deacetylase 6 {AJ011972};

general transcription factor II, i, pseudogene 1 {AI700706}; and

heat shock 70-kD protein 1 {AI700706}.

Examples of genes and/or ESTs of the group (8) involved in cell structure include

H3 histone, family 3B {Z48950}; and

histone deacetylase 6 {AJ011972}.

By individually evaluating the expression level of genes and/or ESTs categorized into the groups (4) to (8) in this manner, it is possible to distinguish how chemical substances having estrogen-like activity affect the cells to be evaluated. In other words, it is preferable that, in the microarrays, the DNA fragments containing a portion or the whole of genes belonging to the groups (4) to (8) are immobilized. In this case, the use of microarrays allows distinction of the types of effects of chemical substances having estrogen-like activity on the cells to be evaluated.

Additionally, a total of 176 kinds of genes and ESTs comprising genes and ESTs of the groups (1), (2), and (3) and positive control genes and such can be categorized into groups of genes that show tissue-specific expression or function. That is, the 176 kinds of genes and ESTs can be categorized into

group (9): genes and/or ESTs functioning in tissues on which estrogen acts,

group (10): genes and/or ESTs characteristic of epithelial cells, and

group (11): genes and/or ESTs functioning in nerve cells.

Specifically, examples of genes and/or ESTs of the group (9) functioning in tissues on which estrogen acts include

trefoil factor 1 {X05030}; and estrogen receptor a {AY033393}.

Examples of genes and/or ESTs of the group (10) characteristic of epithelial cells include

tumor protein D52-like 1 {U44427};

solute carrier family 1, member 4 {L14595};

solute carrier family 12, member 2 {U30246}; and

EGF-containing fibulin-like extracellular matrix protein 1 {U03877}.

Examples of genes and/or ESTs of the group (11) functioning in nerve cells include

amphiregulin {M30704};

neuropeptide Y receptor Y1 {M84755};

synaptogyrin 2 {AJ002308}; and protein tyrosine phosphatase, non-receptor type 18 {X79568}.

By evaluating the expression level of genes categorized into the groups (9) to (11) in this manner, the tissues on which the chemical substances having estrogen-like activity has an effect can be distinguished in the cells to be evaluated.

Furthermore, a total of 176 kinds of genes and ESTs comprising genes and ESTs of the groups (1), (2), and (3) and positive control genes and such may be categorized according to the responsiveness towards tamoxifen, which is an antagonist of estrogen. That is, these 176 kinds of genes and ESTs may be classified into

group (12): genes and/or ESTs whose expression level increases owing to estrogen in tamoxifen-resistant cells, and

group (13): genes and/or ESTs whose expression level decreases owing to estrogen in tamoxifen-resistant cells.

Specifically, examples genes and/or ESTs of the group (12) whose expression level increases owing to estrogen in tamoxifen-resistant cells include

down-regulated in adenoma (DRA) {AI700706}; and

v-jun avian sarcoma virus 17 oncogene homolog {AI598150}.

Examples of genes and/or ESTs of the group (13) whose expression level decreases owing to estrogen in tamoxifen-resistant cells include

carnitine palmitoyltransferase I {L39211};

cadherin 18 {U59325};

insulin-like growth factor binding protein 5 {AA374325}; and Human insulin-like growth factor binding protein 5 {L27560}.

By evaluating the expression levels of genes classified into the groups (12) and (13) in this manner, the effect of chemical substances having estrogen-like activity can be distinguished using tamoxifen-resistant cells as the targets of evaluation.

As described in detail above, this invention provides a microarray that can distinguish the effect of chemical substances having estrogen-like activity on cells to be evaluated. Using this microarray, it is possible to evaluate the effect of environmental hormones and such on cells to be evaluated.

In addition, according to this invention, measurement of the expression levels of genes and/or ESTs in the cells to be evaluated may provide a method for evaluating activity of compounds having estrogen-like activity. The use of this method for evaluating activity of compounds having estrogen-like activity enables evaluation of the effect of environmental hormones and such on the cells to be evaluated.

All the references cited herein are incorporated by reference.

Hereinafter, the use of the microarray of this invention to evaluate the effect of chemical substances having estrogen-like activity in the MCF-7 cell derived tamoxifen resistant strain clone 9 cells will be described as examples. However, this invention is not to be construed as being limited thereto.

### EXAMPLE 1

### The Effect of 17β-estradiol

(1) Preparation of cDNA Sample

In this example, the mRNA extracted from MCF-7 cell-derived tamoxifen-resistant strain clone 9 cells using MACS mRNA separation/purification kit (Miltenyi Biotec) was used as a template to synthesize the labeled cDNA sample.

The labeling reaction was carried out as described below. First, 2 μg of poly (A) RNA and 4.5 μg of oligo(dT) primer were mixed. This was then denatured at 70° C. for 10 minutes and cooled rapidly. Immediately, 6 μl of 5× Superscript buffer (GibcoBRL), 3 μl of DTT, 0.6 μl of 5-× dNTP (Promega), 3 μl of Cy3-dUTP or Cy5-dUTP (Amersham Pharmacia Biotech), and 2 μl of Superscript (GibcoBRL) were added, and reaction was performed for 1 hour at42° C. Again, 2μl of Superscript (GibcoBRL) was added, and reaction was performed for 1 hour at 42° C. Then, 270 μl of TE buffer was added. The reaction solutions to which Cy3-dUTP and Cy5-dUTP, respectively, had been added, were combined and transferred to a single Microcon-30 (MILLIPORE). This was centrifuged at 10,000 rpm until the volume of liquid remaining in the upper cup of Microcon-30 was approximately 10 μl. The solution that passed the cup was transferred to a different tube. To the upper cup of Microcon-30, 500 μl of TE buffer and 15 μg of human Cot1 DNA (GibcoBRL) were added. This was centrifuged at 10,000 rpm until the volume of liquid remaining in the upper cup of Microcon-30 was approximately 10 μl. The upper cup of Microcon-30 was removed and was placed into a new centrifuge tube in an inverted manner. The solution was collected into the centrifuge tube by centrifugation at 3,000 rpm for 3 minutes. To the recovered solution, 10 μg of yeast tRNA (Sigma), 4 μg of poly (dA) (Sigma), and distilled water were added so that the total volume would be 8 μl. To this solution, 1.7 μl of 20×SSC and 0.3 μl of 10% SDS were added, and denaturation was performed for 1 minute at 100° C. By leaving the sample at room temperature for 30 minutes to slowly cool the sample, the cDNA sample containing labeled cDNAs was prepared.

(2) Measurement of Expression Levels Using Microarrays

The cDNA sample prepared in (1) was added dropwise to a microarray in which DNA fragments containing a portion or the whole of the genes and/or ESTs indicated in row “Gene Name” of Table 1 shown below were immobilized on a slide glass (Nisshinbo). The 27 genes of NOs. 5, 6, 59, 60, 64, 75, 83, 90, 91, 92, 98, 103, 104, 112, 113, 114, 121, 126, 128, 138, 148, 155, 159, 176, 184, 195, and 203 in Table 1 are genes constitutively expressing regardless of the presence or absence of estrogen, and the DNA fragments containing a portion or the whole of these genes were immobilized on the basal plate as negative control genes. In addition, changes in the expression levels of the 27 negative control genes and the 176 kinds of genes and ESTs in the presence of 10 nM 17β-estradiol in MCF-7 cells are shown in row “BDE” of Table 1. Here, negative BDE values indicate decrease in expression levels.

After dropwise addition of the cDNA sample onto the DNA microarray, a cover glass (Matsunami) was placed. To prevent drying of the slide glass, the microarray was placed into a hybridization chamber and the chamber was sealed. The hybridization chamber was incubated at 65° C. for 10 to 20 hours. The slide glass was taken out of the hybridization chamber and soaked into 2×SSC/0.2×SDS solution to remove the cover glass. The slide glass was washed with gentle shaking in 1×SSC and was washed again with gentle shaking in 0.2×SSC. After the slide glass was dried, the amount of fluorescence on the DNA microarrays was measured using a DNA microarray fluorescence scanner (Virtek, ChipReader). The results are shown in row “E(example)” of Table 1 shown below. As to genes and ESTs excluding the negative control genes, the weighting factors (row “K” in Table 1) were set based on the respective characteristics. As to the 27 negative control genes, K was taken to be zero.

TABLE 1

| | | | | BDE | | | (evaluation value, | (evaluation value, | | BDE | (evaluation value, | (evaluation value, |
|---|---|---|---|---|---|---|---|---|---|---|---|---|
| No. | Gene Name | GenBank ID | K | (Estrogen) | E(example 1) | BED(example 1) | example 1) | example 1, +K) | E(example 2) | (example 2) | example 2) | example 2, +K) |
| 1 | 17 β-HSD type2 | AF411015 | 1 | 1.17 | 1.01 | 0.95 | 0.09 | 0.09 | 0.41 | 0.60 | 0.29 | 0.29 |
| 2 | A1B1 | AF102108 | 1 | 1.01 | 1.09 | 1.02 | 0.00 | 0.00 | 0.58 | 0.85 | 0.08 | 0.08 |
| 3 | absent in melanoma 1 | U83115 | 1.2 | 0.70 | 0.46 | 0.43 | 0.21 | 0.26 | 1.01 | 1.48 | 0.32 | 0.39 |
| 4 | aconitase 2, | U87939 | 0.5 | 0.94 | 0.82 | 0.77 | 0.08 | 0.04 | 0.55 | 0.80 | 0.07 | 0.03 |
| | mitochondrial | | | | | | | | | | | |
| 5 | actin, alphal, clone | BC003575 | 0 | 1.09 | 1.38 | 1.29 | 0.08 | 0.00 | 0.58 | 0.85 | 0.11 | 0.00 |
| | MGC: 2358 | | | | | | | | | | | |
| 6 | actin, beta | X00351 | 0 | 0.78 | 1.02 | 0.96 | 0.09 | 0.00 | 0.42 | 0.62 | 0.10 | 0.00 |
| 7 | activating transcription | N39944 | 1 | 3.52 | 4.70 | 4.42 | 0.10 | 0.10 | 1.00 | 1.46 | 0.38 | 0.38 |
| | factor 3 | | | | | | | | | | | |
| 8 | AhR | XM040803 | 1 | 1.54 | 1.45 | 1.36 | 0.05 | 0.05 | 0.89 | 1.30 | 0.07 | 0.07 |
| 9 | aldo-keto reductase | T73188 | 0.5 | 0.84 | 0.95 | 0.89 | 0.03 | 0.01 | 0.65 | 0.96 | 0.06 | 0.03 |
| | family 1, member C4 | | | | | | | | | | | |
| | (chlordecone reductase: | | | | | | | | | | | |
| | 3-alpha hydroxysteroid | | | | | | | | | | | |
| | dehydrogenase, type I; | | | | | | | | | | | |
| | dihydrodiol | | | | | | | | | | | |
| | dehydrogenase 4) | | | | | | | | | | | |
| 10 | alpha-1-antichymotrypsin | X00947 | 0.5 | 0.72 | 1.58 | 1.49 | 0.32 | 0.16 | 0.67 | 0.99 | 0.14 | 0.07 |
| 11 | amphiregulin | M30704 | 1 | 2.22 | 2.11 | 1.98 | 0.05 | 0.05 | 0.81 | 1.18 | 0.28 | 0.28 |
| | (schwannoma-derived | | | | | | | | | | | |
| | growth factor) | | | | | | | | | | | |
| 12 | argininosuccinate | AI660571 | 0.5 | 2.58 | 3.39 | 3.19 | 0.09 | 0.05 | 0.64 | 0.93 | 0.44 | 0.22 |
| | synthetase | | | | | | | | | | | |
| 13 | aromatase | AF397171 | 1 | 1.19 | 1.05 | 0.99 | 0.08 | 0.08 | 0.85 | 1.24 | 0.02 | 0.02 |
| 14 | asparagine synthetase | L35946 | 0.5 | 5.69 | 8.39 | 7.88 | 0.14 | 0.07 | 0.88 | 1.28 | 0.65 | 0.32 |
| 15 | B-factor, properdin | X72875 | 0.5 | 1.97 | 1.67 | 1.57 | 0.10 | 0.05 | 0.79 | 1.16 | 0.23 | 0.12 |
| 16 | bassoon (presynaptic | Y18448 | 0.5 | 2.22 | 0.71 | 0.67 | 0.52 | 0.26 | 0.90 | 1.31 | 0.23 | 0.11 |
| | cytomatrix protein) | | | | | | | | | | | |
| 17 | cadherin 18 | U59325 | 1 | 0.77 | 0.58 | 0.54 | 0.15 | 0.15 | 0.75 | 1.10 | 0.16 | 0.16 |
| 18 | calpain, small | AC002984 | 0.5 | 0.47 | 0.42 | 0.40 | 0.07 | 0.04 | 0.47 | 0.69 | 0.16 | 0.08 |
| | polypeptide | | | | | | | | | | | |
| 19 | carnitine | L39211 | 1 | 1.06 | 0.85 | 0.80 | 0.12 | 0.12 | 0.68 | 0.99 | 0.03 | 0.03 |
| | palmitoyltransferase I, | | | | | | | | | | | |
| | liver | | | | | | | | | | | |
| 20 | catenin (cadherin- | U96136 | 1 | 0.54 | 0.39 | 0.37 | 0.17 | 0.17 | 0.64 | 0.94 | 0.24 | 0.24 |
| | associated protein), | | | | | | | | | | | |
| | delta 2 (neural | | | | | | | | | | | |
| | plakophilin-related arm- | | | | | | | | | | | |
| | repeat protein) | | | | | | | | | | | |
| 21 | cathepsin D (lysosomal | M63138 | 0.5 | 1.56 | 1.68 | 1.57 | 0.00 | 0.00 | 0.50 | 0.73 | 0.33 | 0.17 |
| | aspartyl protease) | | | | | | | | | | | |
| 22 | CDC6 (cell division | AF022109 | 1 | 0.96 | 0.80 | 0.75 | 0.11 | 0.11 | 0.68 | 0.99 | 0.01 | 0.01 |
| | cycle 6, S. cerevisiae) | | | | | | | | | | | |
| | homolog | | | | | | | | | | | |
| 23 | CDP-diacylglycerol- | AF014807 | 0.5 | 0.65 | 0.72 | 0.68 | 0.02 | 0.01 | 0.51 | 0.75 | 0.06 | 0.03 |
| | inositol 3- | | | | | | | | | | | |
| | phosphatidyl transferase | | | | | | | | | | | |
| | (phosphatidylinositol | | | | | | | | | | | |
| | synthase) | | | | | | | | | | | |
| 24 | chromobox homolog 1 | AL046741 | 0.5 | 0.69 | 0.67 | 0.63 | 0.04 | 0.02 | 0.70 | 1.02 | 0.17 | 0.08 |
| | (Drosophila HP1 beta) | | | | | | | | | | | |
| 25 | corneodesmosin | L20815 | 0.5 | 0.74 | 0.78 | 0.73 | 0.01 | 0.00 | 0.54 | 0.79 | 0.02 | 0.01 |
| 26 | cyclin A1 | U66838 | 1 | 2.03 | 2.68 | 2.52 | 0.09 | 0.09 | 0.85 | 1.24 | 0.21 | 0.21 |
| 27 | cyclin D | AF318615 | 1 | 1.30 | 1.22 | 1.14 | 0.05 | 0.05 | 0.49 | 0.72 | 0.26 | 0.26 |
| 28 | CYP1A1 | AH007293 | 1 | 1.28 | 1.02 | 0.96 | 0.12 | 0.12 | 42.86 | 62.68 | 1.69 | 1.69 |
| 29 | DKFZP566G223 protein | AI261366 | 0.5 | 2.13 | 2.08 | 1.95 | 0.04 | 0.02 | 0.64 | 0.94 | 0.35 | 0.18 |
| 30 | early growth response 3 | X63741 | 1 | 6.89 | 5.98 | 5.63 | 0.09 | 0.09 | 0.67 | 0.99 | 0.84 | 0.84 |
| 31 | EGF-containing fibulin- | U03877 | 1 | 0.60 | 0.45 | 0.42 | 0.16 | 0.16 | 0.51 | 0.74 | 0.09 | 0.09 |
| | like extracellular | | | | | | | | | | | |
| | matrix protein 1 | | | | | | | | | | | |
| 32 | enolase 2, (gamma, | X51956 | 0.5 | 0.23 | 0.29 | 0.28 | 0.07 | 0.04 | 0.64 | 0.94 | 0.60 | 0.30 |
| | neuronal) | | | | | | | | | | | |
| 33 | enolase 3, (beta, | X56832 | 0.5 | 0.32 | 0.31 | 0.29 | 0.03 | 0.02 | 0.55 | 0.81 | 0.41 | 0.20 |
| | muscle) | | | | | | | | | | | |
| 34 | erbB2 | M86910 | 1 | 0.72 | 0.63 | 0.60 | 0.08 | 0.08 | 0.67 | 0.98 | 0.13 | 0.13 |
| 35 | Estrogen receptor alpha | AY033393 | 1 | 0.55 | 0.48 | 0.45 | 0.09 | 0.09 | 0.42 | 0.61 | 0.04 | 0.04 |
| 36 | Estrogen receptor beta | NM001437 | 1 | 0.91 | 0.87 | 0.82 | 0.05 | 0.05 | 2.79 | 4.08 | 0.65 | 0.65 |
| 37 | Estrogen sulfatase | L25275 | 1 | 1.06 | 0.96 | 0.90 | 0.07 | 0.07 | 1.85 | 2.71 | 0.41 | 0.41 |
| 38 | ESTs(1) | AA587912 | 0.5 | 6.73 | 7.56 | 7.11 | 0.02 | 0.01 | 0.96 | 1.40 | 0.68 | 0.34 |
| 39 | ESTs(2) | AI185199 | 0.5 | 1.39 | 1.49 | 1.40 | 0.00 | 0.00 | 1.22 | 1.78 | 0.11 | 0.05 |
| 40 | ESTs(3) | U82984 | 0.5 | 1.29 | 1.37 | 1.29 | 0.00 | 0.00 | 0.54 | 0.79 | 0.22 | 0.11 |
| 41 | ESTs(4) | X62585 | 0.5 | 2.57 | 2.21 | 2.08 | 0.09 | 0.05 | 0.74 | 1.08 | 0.38 | 0.19 |
| 42 | ESTs(5) | AI949781 | 0.5 | 1.26 | 1.07 | 1.01 | 0.10 | 0.05 | 0.60 | 0.88 | 0.16 | 0.08 |
| 43 | ESTs(6) | AI188401 | 0.5 | 0.64 | 0.60 | 0.56 | 0.05 | 0.03 | 0.72 | 1.05 | 0.22 | 0.11 |
| 44 | ESTs(7) | AI492976 | 0.5 | 0.91 | 0.85 | 0.80 | 0.05 | 0.03 | 0.80 | 1.18 | 0.11 | 0.06 |
| 45 | ESTs(8) | AI767533 | 0.5 | 0.44 | 0.45 | 0.42 | 0.02 | 0.01 | 0.47 | 0.69 | 0.20 | 0.10 |
| 46 | ESTs(9) | N35555 | 0.5 | 3.95 | 9.25 | 8.69 | 0.34 | 0.17 | 1.86 | 2.72 | 0.16 | 0.08 |
| 47 | ESTs(10) | AA975298 | 0.5 | 1.95 | 2.36 | 2.22 | 0.06 | 0.03 | 0.68 | 0.99 | 0.29 | 0.15 |
| 48 | ESTs(11) | AA682502 | 0.5 | 1.36 | 1.08 | 1.02 | 0.13 | 0.06 | 0.52 | 0.76 | 0.25 | 0.13 |
| 49 | ESTs(12) | AI332415 | 0.5 | 1.20 | 1.17 | 1.10 | 0.04 | 0.02 | 0.80 | 1.17 | 0.01 | 0.01 |
| 50 | ESTs(13) | AA629308 | 0.5 | 1.58 | 1.54 | 1.45 | 0.04 | 0.02 | 1.02 | 1.49 | 0.03 | 0.01 |
| 51 | eukaryotic translation | U78525 | 0.5 | 1.51 | 1.48 | 1.39 | 0.03 | 0.02 | 0.66 | 0.97 | 0.19 | 0.10 |
| | initiation factor 3. | | | | | | | | | | | |
| | subunit 9 (eta, 116 kD) | | | | | | | | | | | |
| 52 | exportin, tRNA (nuclear | AF039022 | 0.5 | 2.97 | 3.12 | 2.93 | 0.01 | 0.00 | 1.00 | 1.47 | 0.31 | 0.15 |
| | export receptor for | | | | | | | | | | | |
| | tRNAs) | | | | | | | | | | | |
| 53 | ferritin, heavy | AA102267 | 0.5 | 2.23 | 2.73 | 2.57 | 0.06 | 0.03 | 0.62 | 0.90 | 0.39 | 0.20 |
| | polypeptide 1 | | | | | | | | | | | |
| 54 | FOS-like antigen 2 | X16706 | 1 | 1.56 | 1.98 | 1.86 | 0.08 | 0.08 | 1.10 | 1.61 | 0.01 | 0.01 |
| 55 | FOS-like antigen 2 | AI088306 | 1 | 1.24 | 2.21 | 2.08 | 0.22 | 0.22 | 0.98 | 1.43 | 0.06 | 0.06 |
| 56 | fructose-bisphosphatase | M19922 | 0.5 | 0.40 | 0.45 | 0.42 | 0.03 | 0.01 | 0.60 | 0.88 | 0.34 | 0.17 |
| | 1 | | | | | | | | | | | |
| 57 | fucosyltransferase 8 | Y17977 | 0.5 | 0.75 | 0.56 | 0.53 | 0.15 | 0.08 | 0.65 | 0.94 | 0.10 | 0.05 |
| | (alpha (1,6) | | | | | | | | | | | |
| | fucosyltransferase | | | | | | | | | | | |
| 58 | general transcription | AI700706 | 1 | 0.71 | 0.59 | 0.56 | 0.11 | 0.11 | 0.58 | 0.85 | 0.07 | 0.07 |
| | factor II, i, pseudogene | | | | | | | | | | | |
| | 1 | | | | | | | | | | | |
| 59 | general transcription | AI239815 | 0 | 0.86 | 0.86 | 0.81 | 0.03 | 0.00 | 0.52 | 0.76 | 0.05 | 0.00 |
| | factor IIH, polypeptide | | | | | | | | | | | |
| | 2 (44 kD subunit) | | | | | | | | | | | |
| 60 | glutamate-cysteine | L35546 | 0 | 0.69 | 0.86 | 0.81 | 0.07 | 0.00 | 0.49 | 0.72 | 0.02 | 0.00 |
| | ligase (gamma- | | | | | | | | | | | |
| | glutamylcysteine | | | | | | | | | | | |
| | synthetase), regulatory | | | | | | | | | | | |
| | (30.8 kD) | | | | | | | | | | | |
| 61 | glutamic-oxaloacetic | M37400 | 0.5 | 1.97 | 2.52 | 2.36 | 0.08 | 0.04 | 0.90 | 1.32 | 0.17 | 0.09 |
| | transaminase 1, soluble | | | | | | | | | | | |
| | (aspartate | | | | | | | | | | | |
| | aminotransferase 1) | | | | | | | | | | | |
| 62 | glutamine-fructose-6- | M90516 | 0.5 | 3.91 | 3.44 | 3.24 | 0.08 | 0.04 | 0.73 | 1.07 | 0.56 | 0.28 |
| | phosphate transaminase 1 | | | | | | | | | | | |
| 63 | glycyl-tRNA synthetase | D30658 | 0.5 | 1.92 | 2.22 | 2.09 | 0.04 | 0.02 | 0.80 | 1.17 | 0.21 | 0.11 |
| 64 | H. sapiens tunp mRNA for | X72727 | 0 | 1.29 | 1.32 | 1.24 | 0.02 | 0.00 | 0.59 | 0.87 | 0.17 | 0.00 |
| | transformation | | | | | | | | | | | |
| | upregulated nuclear | | | | | | | | | | | |
| | protein | | | | | | | | | | | |
| 65 | H. sapiens SCA1 mRNA for | X79203 | 1 | 2.05 | 2.14 | 2.01 | 0.01 | 0.01 | 1.12 | 1.64 | 0.10 | 0.10 |
| | ataxin | | | | | | | | | | | |
| 66 | H3 histone, family 3B | Z48950 | 1 | 1.86 | 1.85 | 1.74 | 0.03 | 0.03 | 0.45 | 0.66 | 0.45 | 0.45 |
| | (H3. 3B) | | | | | | | | | | | |
| 67 | heat shock 70 kD protein | M59828 | 1 | 0.56 | 0.54 | 0.50 | 0.05 | 0.05 | 0.68 | 1.00 | 0.25 | 0.25 |
| | 1 | | | | | | | | | | | |
| 68 | heat shock 70 kD protein | AI878886 | 0.5 | 4.32 | 4.22 | 3.97 | 0.04 | 0.02 | 0.57 | 0.83 | 0.72 | 0.36 |
| | 5 (glucose-regulated | | | | | | | | | | | |
| | protein, 78 kD) | | | | | | | | | | | |
| 69 | histone deacetylase 6 | AJ011972 | 1 | 0.72 | 0.70 | 0.66 | 0.04 | 0.04 | 0.56 | 0.82 | 0.06 | 0.06 |
| 70 | Homo sapiens adipose | BC004471 | 1 | 0.38 | 0.39 | 0.37 | 0.02 | 0.02 | 0.81 | 1.18 | 0.49 | 0.49 |
| | specific 2, clone | | | | | | | | | | | |
| | MGC: 10343, mRNA | | | | | | | | | | | |
| | complete. | | | | | | | | | | | |
| 71 | Homo sapiens BAC clone | AC005192 | 1 | 2.69 | 2.29 | 2.15 | 0.10 | 0.10 | 0.80 | 1.17 | 0.36 | 0.36 |
| | CTB-163k11 from 7q31, | | | | | | | | | | | |
| | complete cds. | | | | | | | | | | | |
| 72 | Homo sapiens ccr2b | AF012281 | 0.5 | 2.13 | 1.52 | 1.43 | 0.17 | 0.09 | 0.96 | 1.40 | 0.18 | 0.09 |
| | (ccr2), ccr2a (ccr2), | | | | | | | | | | | |
| | ccr5 (ccr5) and ccr6 | | | | | | | | | | | |
| | (ccr6) genes, complete | | | | | | | | | | | |
| | cds, and lactoferrin | | | | | | | | | | | |
| | (lactoferrin) | | | | | | | | | | | |
| | gene, partial cds, | | | | | | | | | | | |
| | complete sequence | | | | | | | | | | | |
| 73 | Homo sapiens CDC14 (cell | XM-011585 | 1 | 1.00 | 1.02 | 0.96 | 0.02 | 0.02 | 0.87 | 1.27 | 0.11 | 0.11 |
| | division cycle 14, S. | | | | | | | | | | | |
| | cerevisiae) homolog B | | | | | | | | | | | |
| | (CDC14B), mRNA | | | | | | | | | | | |
| 74 | Homo sapiens cDNA | AK022110 | 1 | 1.02 | 1.15 | 1.08 | 0.03 | 0.03 | 0.78 | 1.14 | 0.05 | 0.05 |
| | FLJ12048fis, clone | | | | | | | | | | | |
| | HEMBB1001990 | | | | | | | | | | | |
| 75 | Homo sapiens cDNA | AK0022810 | 0 | 1.85 | 1.86 | 1.75 | 0.02 | 0.00 | 0.71 | 1.03 | 0.25 | 0.00 |
| | FLJ12748fis, clone | | | | | | | | | | | |
| | NT2RP2001137 | | | | | | | | | | | |
| 76 | Homo sapiens cDNA | AK022810 | 1 | 1.20 | 1.03 | 0.97 | 0.09 | 0.09 | 3.07 | 4.49 | 0.57 | 0.57 |
| | FLJ12748fis, clone | | | | | | | | | | | |
| | NT2RP2001137 | | | | | | | | | | | |
| 77 | Homo sapiens chromosome | AC004526 | 1 | 0.71 | 0.48 | 0.45 | 0.20 | 0.20 | 0.76 | 1.11 | 0.19 | 0.19 |
| | 17, Neurofibromatosis 1 | | | | | | | | | | | |
| | locus, complete sequence | | | | | | | | | | | |
| 78 | Homo sapiens clone 23783 | AF054996 | 0.5 | 0.74 | 0.71 | 0.67 | 0.04 | 0.02 | 0.63 | 0.92 | 0.09 | 0.05 |
| | mRNA sequence | | | | | | | | | | | |
| 79 | Homo sapiens DNA from | AC004030 | 0.5 | 0.46 | 0.41 | 0.39 | 0.08 | 0.04 | 0.57 | 0.84 | 0.26 | 0.13 |
| | chromosome 19, cosmid | | | | | | | | | | | |
| | F21856 | | | | | | | | | | | |
| 80 | Homo sapiens integrin- | XM_006424 | 1 | 2.79 | 1.08 | 1.02 | 0.44 | 0.44 | 1.17 | 1.71 | 0.21 | 0.21 |
| | linked kinase (ILK), | | | | | | | | | | | |
| | mRNA | | | | | | | | | | | |
| 81 | Homo sapiens integrin- | XM-006424 | 1 | 1.12 | 1.17 | 1.10 | 0.01 | 0.01 | 0.59 | 0.86 | 0.11 | 0.11 |
| | linked kinase (ILK), | | | | | | | | | | | |
| | mRNA | | | | | | | | | | | |
| 82 | Homo sapiens interleukin | XM-009962 | 1 | 0.89 | 1.26 | 1.18 | 0.13 | 0.13 | 0.53 | 0.78 | 0.06 | 0.06 |
| | 2 receptor, beta | | | | | | | | | | | |
| | (IL2RB), mRNA | | | | | | | | | | | |
| 83 | Homo sapiens interleukin | NM002184 | 0 | 1.62 | 2.17 | 2.04 | 0.10 | 0.00 | 0.75 | 1.09 | 0.17 | 0.00 |
| | 6 signal transducer | | | | | | | | | | | |
| | (gp130, oncostatin M | | | | | | | | | | | |
| | receptor) (IL6ST), mRNA | | | | | | | | | | | |
| 84 | Homo sapiens methylene | XM_002234 | 1 | 5.94 | 6.85 | 6.44 | 0.03 | 0.03 | 0.72 | 1.06 | 0.75 | 0.75 |
| | tetrahydrofolate | | | | | | | | | | | |
| | dehydrogenase (NAD + | | | | | | | | | | | |
| | dependent), | | | | | | | | | | | |
| | methenyltetrahydrofolate | | | | | | | | | | | |
| | cyclohydrolase(MTHFD2), | | | | | | | | | | | |
| | mRNA | | | | | | | | | | | |
| 85 | Homo sapiens mRNA for | D86979 | 1 | 0.90 | 0.89 | 0.84 | 0.03 | 0.03 | 0.71 | 1.04 | 0.06 | 0.06 |
| | KIAA0226 protein, | | | | | | | | | | | |
| | partial cds | | | | | | | | | | | |
| 86 | Homo sapiens mRNA for | X76732 | 1 | 2.46 | 1.82 | 1.71 | 0.16 | 0.16 | 0.73 | 1.07 | 0.36 | 0.36 |
| | NEFA protein | | | | | | | | | | | |
| 87 | Homo sapiens mRNA full | M60922 | 1 | 0.73 | 0.70 | 0.66 | 0.04 | 0.04 | 0.61 | 0.89 | 0.09 | 0.09 |
| | length insert cDNA clone | | | | | | | | | | | |
| | EUROIMAGE781354 | | | | | | | | | | | |
| 88 | Homo sapiens paired | NM-002570 | 1 | 0.66 | 0.67 | 0.63 | 0.02 | 0.02 | 0.65 | 0.96 | 0.16 | 0.16 |
| | basic amino acid | | | | | | | | | | | |
| | cleaving system 4 | | | | | | | | | | | |
| | (PACE4), mRNA | | | | | | | | | | | |
| 89 | Homo sapiens partial | AJ276317 | 1 | 3.54 | 4.44 | 4.17 | 0.07 | 0.07 | 1.21 | 1.77 | 0.30 | 0.30 |
| | mRNA for A-type | | | | | | | | | | | |
| | potassium channel | | | | | | | | | | | |
| | modulatory protein 2 | | | | | | | | | | | |
| | (KCHIP2 gene), 3′ UTR | | | | | | | | | | | |
| 90 | Homo sapiens protein | XM003106 | 0 | 0.70 | 0.50 | 0.47 | 0.18 | 0.00 | 0.74 | 1.09 | 0.19 | 0.00 |
| | kinase C delta(PRKCD) | | | | | | | | | | | |
| | mRNA | | | | | | | | | | | |
| 91 | Homo sapiens protein | XM009395 | 0 | 0.86 | 0.74 | 0.69 | 0.09 | 0.00 | 0.58 | 0.85 | 0.00 | 0.00 |
| | kinase related to | | | | | | | | | | | |
| | S.cerevisiae STE20, | | | | | | | | | | | |
| | effector for | | | | | | | | | | | |
| | cde42HS(PAK4), mRNA | | | | | | | | | | | |
| 92 | Homo sapiens putative | XM005791 | 0 | 0.88 | 0.59 | 0.56 | 0.20 | 0.00 | 0.92 | 1.35 | 0.18 | 0.00 |
| | transmembrane | | | | | | | | | | | |
| | protein(NMA), mRNA | | | | | | | | | | | |
| 93 | Homo sapiens quiescin Q6 | NM-002826 | 1 | 0.73 | 0.67 | 0.63 | 0.06 | 0.06 | 1.04 | 1.52 | 0.32 | 0.32 |
| | (QSCN6), mRNA | | | | | | | | | | | |
| 94 | Homo sapiens ribosormal | NM000993 | 1 | 0.63 | 0.64 | 0.61 | 0.02 | 0.02 | 0.71 | 1.04 | 0.22 | 0.22 |
| | protein L31(RPL31), mRNA | | | | | | | | | | | |
| 95 | Homo sapiens ryanodine | XM-001778 | 0.5 | 1.16 | 1.06 | 1.00 | 0.06 | 0.03 | 1.07 | 1.57 | 0.13 | 0.07 |
| | receptor 2 (cardiac) | | | | | | | | | | | |
| | (RYR2), mRNA | | | | | | | | | | | |
| 96 | Homo sapiens similar to | XM_002923 | 1 | 2.20 | 2.36 | 2.22 | 0.00 | 0.00 | 0.65 | 0.95 | 0.36 | 0.36 |
| | chemokine (C—C motif) | | | | | | | | | | | |
| | receptor 2 (H. sapiens) | | | | | | | | | | | |
| | (LOC82514), mRNA | | | | | | | | | | | |
| 97 | Homo sapiens solute | L02785 | 1 | 1.15 | 1.24 | 1.17 | 0.01 | 0.01 | 0.90 | 1.32 | 0.06 | 0.06 |
| | carrier family 26, | | | | | | | | | | | |
| | member 3 (SLC26A3), mRNA | | | | | | | | | | | |
| 98 | Homo sapiens STAT- | AF037989 | 0 | 0.91 | 0.67 | 0.63 | 0.16 | 0.00 | 2.60 | 3.80 | 0.62 | 0.00 |
| | induced STAT inhibitor-2 | | | | | | | | | | | |
| | mRNA | | | | | | | | | | | |
| 99 | Homo sapiens | NM-006756 | 1 | 1.71 | 1.59 | 1.49 | 0.06 | 0.06 | 0.67 | 0.98 | 0.24 | 0.24 |
| | transcription elongation | | | | | | | | | | | |
| | factor A (SII), 1 | | | | | | | | | | | |
| | (TCEA1), mRNA | | | | | | | | | | | |
| 100 | Homo sapiens | XM002732 | 1 | 1.52 | 1.38 | 1.30 | 0.07 | 0.07 | 2.17 | 3.17 | 0.32 | 0.32 |
| | transforming growth | | | | | | | | | | | |
| | factor, alpha(TGFA), mRNA | | | | | | | | | | | |
| 101 | Homo sapiens UDP- | NM001497 | 1 | 1.02 | 0.87 | 0.82 | 0.10 | 0.10 | 0.46 | 0.68 | 0.18 | 0.18 |
| | Gal:betaGlcNAc beta 1,4- | | | | | | | | | | | |
| | galactocyltransferase, po | | | | | | | | | | | |
| | lypeptide1(B4GALT1), mRNA | | | | | | | | | | | |
| 102 | Homo sapiens vascular | AF024710 | 1 | 1.61 | 1.86 | 1.75 | 0.04 | 0.04 | 1.21 | 1.76 | 0.04 | 0.04 |
| | endothelial growth | | | | | | | | | | | |
| | factor(VEGF) mRNA, 3′ UTR | | | | | | | | | | | |
| 103 | Homo sapiens , short- | BC002730 | 0 | 1.48 | 1.50 | 1.41 | 0.02 | 0.00 | 1.24 | 1.81 | 0.09 | 0.00 |
| | chain | | | | | | | | | | | |
| | deghydrogenase/reductase | | | | | | | | | | | |
| | 1, clone MGC3784, mRNA | | | | | | | | | | | |
| 104 | Homo sapiens , TLS- | BC005039 | 0 | 0.84 | 0.97 | 0.91 | 0.04 | 0.00 | 0.56 | 0.82 | 0.01 | 0.00 |
| | associated serine- | | | | | | | | | | | |
| | arginine protein2, clone | | | | | | | | | | | |
| | MGC:12764, mRNA | | | | | | | | | | | |
| 105 | Homo sapinens mRNA; cDNA | AL137638 | 1 | 0.64 | 0.32 | 0.30 | 0.32 | 0.32 | 0.49 | 0.72 | 0.05 | 0.05 |
| | DKFZp434J065(from clone | | | | | | | | | | | |
| | DKFZp434J065) | | | | | | | | | | | |
| 106 | HS1 binding protein | N28312 | 0.5 | 1.50 | 1.87 | 1.76 | 0.07 | 0.03 | 0.58 | 0.85 | 0.25 | 0.12 |
| 107 | Human 150 kDa oxgen- | U65785 | 1 | 2.09 | 2.58 | 2.43 | 0.06 | 0.06 | 0.80 | 1.17 | 0.25 | 0.25 |
| | regulated protein ORP150 | | | | | | | | | | | |
| | mRNA, complete. | | | | | | | | | | | |
| 108 | Human autotaxin-t(atx- | L46720 | 1 | 1.01 | 0.62 | 0.58 | 0.24 | 0.24 | 1.62 | 2.37 | 0.37 | 0.37 |
| | t)gene, complete code. | | | | | | | | | | | |
| 109 | Human breast cancer | U43944 | 1 | 1.88 | 2.19 | 2.06 | 0.04 | 0.04 | 1.11 | 1.63 | 0.06 | 0.06 |
| | cytosolic NADP(+)- | | | | | | | | | | | |
| | dependent malic enzyme | | | | | | | | | | | |
| | mRNA, partial cds | | | | | | | | | | | |
| 110 | Human chromosome 14 DNA | AL118556 | 1 | 1.25 | 1.38 | 1.30 | 0.02 | 0.02 | 1.08 | 1.58 | 0.10 | 0.10 |
| | sequence BAC R-406H23oq | | | | | | | | | | | |
| | library | | | | | | | | | | | |
| 111 | Human clone 23855 mRNA, | U79302 | 0.5 | 1.27 | 0.83 | 0.78 | 0.21 | 0.11 | 0.62 | 0.91 | 0.14 | 0.07 |
| | partial cds | | | | | | | | | | | |
| 112 | Human DNA sequence from | Z98941 | 0 | 1.20 | 1.02 | 0.96 | 0.10 | 0.00 | 0.90 | 1.32 | 0.04 | 0.00 |
| | PAC 204E5 on chromosome | | | | | | | | | | | |
| | 12. Contains exon | | | | | | | | | | | |
| | similar to Wilms' | | | | | | | | | | | |
| | Tumour-related protein | | | | | | | | | | | |
| | QM-like P2X-like | | | | | | | | | | | |
| | receptor, ATP ligand | | | | | | | | | | | |
| | gated ion channel, ESTs, | | | | | | | | | | | |
| | CpG island | | | | | | | | | | | |
| 113 | Human GOS2 gene, 5′ | M72855 | 0 | 1.20 | 1.13 | 1.06 | 0.05 | 0.00 | 1.00 | 1.47 | 0.09 | 0.00 |
| | flank and cds | | | | | | | | | | | |
| 114 | Human GT mitochondrial | M31659 | 0 | 0.60 | 0.50 | 0.47 | 0.11 | 0.00 | 0.69 | 1.01 | 0.22 | 0.00 |
| | solute carrier protein | | | | | | | | | | | |
| 115 | Human inositol 1,3,4- | U51335 | 1 | 1.47 | 1.51 | 1.42 | 0.02 | 0.02 | 0.65 | 0.95 | 0.19 | 0.19 |
| | trisphosphate 5/6-kinase | | | | | | | | | | | |
| | mRNA, complete cds | | | | | | | | | | | |
| 116 | Human insulin-like | L27560 | 1 | 0.60 | 0.67 | 0.63 | 0.02 | 0.02 | 0.74 | 1.08 | 0.26 | 0.26 |
| | growth factor binding | | | | | | | | | | | |
| | protein 5 (IGFBP5) mRNA | | | | | | | | | | | |
| 117 | Human intercrine-alpha | U19495 | 1 | 0.99 | 1.33 | 1.25 | 0.10 | 0.10 | 0.68 | 1.00 | 0.00 | 0.00 |
| | (hIRH) mRNA, complete | | | | | | | | | | | |
| | cds | | | | | | | | | | | |
| 118 | Human low-Mr GTP-binding | U59878 | 1 | 1.73 | 2.23 | 2.10 | 0.09 | 0.09 | 0.74 | 1.08 | 0.20 | 0.20 |
| | protein(RAB32)mRNA, | | | | | | | | | | | |
| | partial cds | | | | | | | | | | | |
| 119 | Human meningioma- | U73682 | 1 | 2.40 | 2.27 | 2.13 | 0.05 | 0.05 | 0.80 | 1.17 | 0.31 | 0.31 |
| | expressed antigen | | | | | | | | | | | |
| | II(MEAII) mRNA, partial | | | | | | | | | | | |
| | cds. | | | | | | | | | | | |
| 120 | Human mRNA amd promoter | X51730 | 1 | 1.94 | 2.04 | 1.92 | 0.00 | 0.00 | 0.76 | 1.11 | 0.24 | 0.24 |
| | DNA for progesteron | | | | | | | | | | | |
| | receptor | | | | | | | | | | | |
| 121 | Human mRNA for glucose- | X03673 | 0 | 1.26 | 1.32 | 1.24 | 0.01 | 0.00 | 0.77 | 1.13 | 0.05 | 0.00 |
| | 6-phosphate | | | | | | | | | | | |
| | dehydrogenase (G6PD). | | | | | | | | | | | |
| 122 | Human mRNA for KIAA0008 | D13633 | 1 | 1.64 | 1.83 | 1.72 | 0.02 | 0.02 | 0.62 | 0.90 | 0.26 | 0.26 |
| 123 | Human mRNA for KIAA0018 | D13643 | 0.5 | 1.38 | 1.62 | 1.52 | 0.04 | 0.02 | 0.50 | 0.73 | 0.27 | 0.14 |
| | gene, complete cds | | | | | | | | | | | |
| 124 | Human mRNA for KIAA0062 | D31887 | 1 | 2.11 | 1.96 | 1.84 | 0.06 | 0.06 | 0.73 | 1.07 | 0.30 | 0.30 |
| | gene, partial cds | | | | | | | | | | | |
| 125 | Human mRNA for KIAA0101 | D14656 | 1 | 0.62 | 0.72 | 0.68 | 0.04 | 0.04 | 0.55 | 0.80 | 0.11 | 0.11 |
| | gene, complete cds | | | | | | | | | | | |
| 126 | Human mRNA for KIAA0349 | AB002347 | 0 | 1.12 | 1.04 | 0.98 | 0.06 | 0.00 | 0.85 | 1.25 | 0.05 | 0.00 |
| | gene, partial | | | | | | | | | | | |
| 127 | Human mRNA for KIAA0373 | AB002371 | 1 | 0.58 | 0.67 | 0.63 | 0.03 | 0.03 | 0.98 | 1.44 | 0.39 | 0.39 |
| | gene, complete cds | | | | | | | | | | | |
| 128 | Human pre-B cell | L36033 | 0 | 0.99 | 0.68 | 0.64 | 0.19 | 0.00 | 0.90 | 1.31 | 0.12 | 0.00 |
| | stimulating factor | | | | | | | | | | | |
| | homologue (SDF1b)mRNA, | | | | | | | | | | | |
| | complete | | | | | | | | | | | |
| 129 | Human threonyl-tRNA | M63180 | 1 | 2.46 | 2.32 | 2.18 | 0.05 | 0.05 | 0.83 | 1.21 | 0.31 | 0.31 |
| | synthetase mRNA, | | | | | | | | | | | |
| | complete | | | | | | | | | | | |
| 130 | Human transcripton | U85658 | 1 | 1.51 | 1.63 | 1.53 | 0.01 | 0.01 | 0.68 | 1.00 | 0.18 | 0.18 |
| | factor ERF-1 mRNA, | | | | | | | | | | | |
| | complete | | | | | | | | | | | |
| 131 | hyaluronan-mediated | U29343 | 1.5 | 2.53 | 2.27 | 2.14 | 0.07 | 0.11 | 0.72 | 1.05 | 0.38 | 0.57 |
| | motility receptor | | | | | | | | | | | |
| | (RHAMM) | | | | | | | | | | | |
| 132 | IGFR | JQ1035 | 1 | 1.13 | 1.07 | 1.00 | 0.05 | 0.05 | 0.74 | 1.08 | 0.02 | 0.02 |
| 133 | inhibitor of DNA binding | AL022726 | 0.5 | 0.51 | 0.51 | 0.48 | 0.02 | 0.01 | 0.55 | 0.81 | 0.20 | 0.10 |
| | 4, dominant negative | | | | | | | | | | | |
| | helix-loop-helix protein | | | | | | | | | | | |
| 134 | insulin-like growth | AA374325 | 1 | 0.69 | 0.33 | 0.31 | 0.35 | 0.35 | 0.56 | 0.82 | 0.08 | 0.08 |
| | factor binding protein 5 | | | | | | | | | | | |
| 135 | insulin-like growth | M62403 | 1 | 3.25 | 4.53 | 4.26 | 0.12 | 0.12 | 0.52 | 0.77 | 0.63 | 0.63 |
| | factor-binding protein 4 | | | | | | | | | | | |
| 136 | interferon stimulated | X89773 | 0.5 | 1.75 | 2.35 | 2.20 | 0.10 | 0.05 | 0.58 | 0.85 | 0.31 | 0.16 |
| | gene (20 kD) | | | | | | | | | | | |
| 137 | isoleucine-tRNA | D28473 | 0.5 | 0.85 | 0.90 | 0.85 | 0.00 | 0.00 | 0.52 | 0.76 | 0.05 | 0.02 |
| | synthetase | | | | | | | | | | | |
| 138 | keratin 68 | L42611 | 0 | 1.64 | 1.62 | 1.52 | 0.03 | 0.00 | 0.47 | 0.68 | 0.38 | 0.00 |
| 139 | keratin 8 | AI273887 | 0.5 | 1.22 | 1.34 | 1.26 | 0.01 | 0.01 | 0.42 | 0.62 | 0.29 | 0.15 |
| 140 | KIAA0058 gene product | AL036958 | 0.5 | 0.98 | 0.79 | 0.74 | 0.12 | 0.06 | 0.45 | 0.66 | 0.18 | 0.09 |
| 141 | KIAA0123 protein | D21064 | 0.5 | 0.81 | 0.73 | 0.68 | 0.07 | 0.04 | 0.71 | 1.04 | 0.11 | 0.05 |
| 142 | KIAA0172 | D79994 | 0.5 | 0.91 | 0.64 | 0.60 | 0.18 | 0.09 | 0.72 | 1.05 | 0.06 | 0.03 |
| 143 | KIAA0196 gene product | D83780 | 0.5 | 0.82 | 0.72 | 0.67 | 0.09 | 0.04 | 0.65 | 0.95 | 0.06 | 0.03 |
| 144 | KIAA0307 gene product | AB002305 | 0.5 | 0.60 | 0.72 | 0.68 | 0.05 | 0.03 | 0.77 | 1.12 | 0.27 | 0.14 |
| 145 | KIAA0551 protein | AB011123 | 0.5 | 1.06 | 1.12 | 1.05 | 0.01 | 0.00 | 0.58 | 0.84 | 0.10 | 0.05 |
| 146 | KIAA0587 gene product | AB011159 | 0.5 | 1.59 | 1.65 | 1.55 | 0.01 | 0.01 | 0.72 | 1.06 | 0.18 | 0.09 |
| 147 | KIAA1051 protein | AB028974 | 0.5 | 1.41 | 1.40 | 1.32 | 0.03 | 0.01 | 0.80 | 1.18 | 0.08 | 0.04 |
| 148 | Macaca fascicularis | AB050427 | 0 | 1.01 | 1.24 | 1.17 | 0.06 | 0.00 | 3.83 | 5.60 | 0.75 | 0.00 |
| | brain cDNA, clone:QnpA- | | | | | | | | | | | |
| | 21739 | | | | | | | | | | | |
| 149 | mal, T-cell | X76220 | 0.5 | 1.01 | 1.01 | 0.94 | 0.03 | 0.02 | 1.67 | 2.44 | 0.38 | 0.19 |
| | differentiation protein | | | | | | | | | | | |
| 150 | mannosidase, alpha, | X74837 | 0.5 | 1.56 | 1.37 | 1.29 | 0.08 | 0.04 | 0.84 | 1.23 | 0.10 | 0.05 |
| | class 1A, member 1 | | | | | | | | | | | |
| 151 | matrix Gla protein | AA484893 | 0.5 | 3.14 | 4.78 | 4.49 | 0.16 | 0.08 | 0.36 | 0.53 | 0.77 | 0.39 |
| 152 | membrane component, | X13425 | 0.5 | 0.44 | 0.44 | 0.41 | 0.03 | 0.01 | 0.45 | 0.65 | 0.17 | 0.09 |
| | chromosome 1, surface | | | | | | | | | | | |
| | marker 1 (40 kD | | | | | | | | | | | |
| | glycoprotein, identified | | | | | | | | | | | |
| | by monoclonal antibody | | | | | | | | | | | |
| | GA733) | | | | | | | | | | | |
| 153 | methylene | X16396 | 0.5 | 3.26 | 3.15 | 2.96 | 0.04 | 0.02 | 0.63 | 0.92 | 0.55 | 0.27 |
| | tetrahydrofolate | | | | | | | | | | | |
| | dehydrogenase (NAD + | | | | | | | | | | | |
| | dependent), | | | | | | | | | | | |
| | methenyltetrahydrofolate | | | | | | | | | | | |
| | cyclohydrolase {Incyte | | | | | | | | | | | |
| | PD: 2455118} | | | | | | | | | | | |
| 154 | microtubule-associated | L06237 | 0.5 | 1.27 | 1.19 | 1.12 | 0.06 | 0.03 | 0.83 | 1.21 | 0.02 | 0.01 |
| | protein 1B | | | | | | | | | | | |
| 155 | mRNA from KIAA 1151 | AB032977 | 0 | 1.34 | 1.22 | 1.15 | 0.07 | 0.00 | 1.84 | 2.68 | 0.30 | 0.00 |
| | protein, partial. | | | | | | | | | | | |
| 156 | MYC promoter-binding | AA482422 | 1.2 | 0.25 | 0.29 | 0.28 | 0.04 | 0.05 | 0.46 | 0.67 | 0.43 | 0.52 |
| | protein 1 | | | | | | | | | | | |
| 157 | neuropeptide Y receptor | M84755 | 1.5 | 0.84 | 0.99 | 0.93 | 0.04 | 0.06 | 0.75 | 1.10 | 0.12 | 0.18 |
| | Y1 | | | | | | | | | | | |
| 158 | nuclear receptor | X84373 | 1.5 | 2.14 | 2.40 | 2.25 | 0.02 | 0.03 | 0.81 | 1.19 | 0.25 | 0.38 |
| | interacting protein 1 | | | | | | | | | | | |
| 159 | nucleophosmin (nucleolar | AA173870 | 0 | 0.69 | 0.89 | 0.83 | 0.09 | 0.00 | 0.79 | 1.15 | 0.22 | 0.00 |
| | phosphoprotein B23, | | | | | | | | | | | |
| | numatrin) | | | | | | | | | | | |
| 160 | paired basic amino acid | D87993 | 0.5 | 0.72 | 0.76 | 0.71 | 0.01 | 0.00 | 0.55 | 0.81 | 0.05 | 0.03 |
| | cleaving system 4 | | | | | | | | | | | |
| 161 | phorbol-12-myristate-13- | D90070 | 1.2 | 1.21 | 1.48 | 1.40 | 0.06 | 0.08 | 0.69 | 1.00 | 0.08 | 0.10 |
| | acetate-induced protein | | | | | | | | | | | |
| | 1 | | | | | | | | | | | |
| 162 | phosphoenolpyruvate | X92720 | 0.5 | 3.80 | 6.80 | 6.39 | 0.23 | 0.11 | 0.56 | 0.81 | 0.67 | 0.34 |
| | carboxykinase 2 | | | | | | | | | | | |
| | (mitochondrial) | | | | | | | | | | | |
| 163 | phosphoinositide-3- | Z46973 | 0.5 | 1.16 | 0.97 | 0.91 | 0.10 | 0.05 | 1.56 | 2.28 | 0.29 | 0.15 |
| | kinase, class 3 | | | | | | | | | | | |
| 164 | poliovirus receptor | X64116 | 1.5 | 0.71 | 1.11 | 1.04 | 0.17 | 0.25 | 0.71 | 1.04 | 0.17 | 0.25 |
| 165 | progesterone receptor | XP006190 | 1 | 0.86 | 0.96 | 0.90 | 0.02 | 0.02 | 0.96 | 1.41 | 0.21 | 0.21 |
| 166 | prostate differentiation | AA216685 | 1 | 1.67 | 1.97 | 1.85 | 0.05 | 0.05 | 0.89 | 1.30 | 0.11 | 0.11 |
| | factor | | | | | | | | | | | |
| 167 | protein | AA481712 | 0.5 | 1.68 | 1.83 | 1.72 | 0.01 | 0.01 | 0.45 | 0.66 | 0.40 | 0.20 |
| | geranylgeranyltransferas | | | | | | | | | | | |
| | e type I, beta subunit | | | | | | | | | | | |
| 168 | protein kinase C | J03075 | 0.5 | 0.80 | 0.62 | 0.58 | 0.14 | 0.07 | 0.57 | 0.83 | 0.02 | 0.01 |
| | substrate 80K-H | | | | | | | | | | | |
| 169 | protein kinase C, delta | D10495 | 0.5 | 1.80 | 2.24 | 2.11 | 0.07 | 0.03 | 0.58 | 0.85 | 0.33 | 0.16 |
| 170 | protein tyrosine | X79568 | 1 | 0.79 | 0.81 | 0.76 | 0.01 | 0.01 | 0.61 | 0.90 | 0.06 | 0.06 |
| | phosphatase, non- | | | | | | | | | | | |
| | receptor type 18 (brain- | | | | | | | | | | | |
| | derived) | | | | | | | | | | | |
| 171 | quiescin Q6 | U97276 | 0.5 | 0.43 | 0.43 | 0.40 | 0.03 | 0.02 | 0.74 | 1.08 | 0.40 | 0.20 |
| 172 | ras homolog gene family, | L25081 | 1.2 | 1.00 | 0.94 | 0.88 | 0.05 | 0.07 | 0.46 | 0.68 | 0.17 | 0.20 |
| | member C | | | | | | | | | | | |
| 173 | reticulocalbin 1, EF- | D42073 | 0.5 | 1.92 | 2.33 | 2.19 | 0.06 | 0.03 | 0.80 | 1.17 | 0.22 | 0.11 |
| | hand calcium binding | | | | | | | | | | | |
| | domain | | | | | | | | | | | |
| 174 | retinoblastoma-binding | U72066 | 1.2 | 1.56 | 3.14 | 2.95 | 0.28 | 0.33 | 1.56 | 2.28 | 0.17 | 0.20 |
| | protein 8 | | | | | | | | | | | |
| 175 | Rho GDP dissociation | X69550 | 0.5 | 0.44 | 0.48 | 0.45 | 0.01 | 0.01 | 0.48 | 0.70 | 0.20 | 0.10 |
| | inhibitor (GDI) alpha | | | | | | | | | | | |
| 176 | ribosomal protein L35 | AI815757 | 0 | 0.68 | 0.84 | 0.79 | 0.06 | 0.00 | 0.50 | 0.74 | 0.03 | 0.00 |
| 177 | ribosomal protein S6 | U08316 | 1 | 1.43 | 1.85 | 1.74 | 0.08 | 0.08 | 0.78 | 1.13 | 0.10 | 0.10 |
| | kinase, 90 kD, | | | | | | | | | | | |
| | polypeptide 3 | | | | | | | | | | | |
| 178 | S100 calcium-binding | AI151190 | 0.5 | 2.27 | 2.34 | 2.20 | 0.01 | 0.01 | 1.13 | 1.66 | 0.14 | 0.07 |
| | protein P | | | | | | | | | | | |
| 179 | selenium binding protein | U29091 | 0.5 | 0.68 | 0.47 | 0.45 | 0.18 | 0.09 | 0.43 | 0.63 | 0.03 | 0.02 |
| | 1 | | | | | | | | | | | |
| 180 | serine | U23143 | 0.5 | 2.59 | 3.34 | 3.14 | 0.08 | 0.04 | 0.63 | 0.92 | 0.45 | 0.22 |
| | hydroxymethyltransferase | | | | | | | | | | | |
| | 2 (mitochondrial) | | | | | | | | | | | |
| 181 | SH3-domain binding | AB005047 | 0.5 | 2.14 | 2.41 | 2.27 | 0.03 | 0.01 | 0.58 | 0.85 | 0.40 | 0.20 |
| | protein 5 (BTK- | | | | | | | | | | | |
| | associated) | | | | | | | | | | | |
| 182 | solute carrier family 1 | AF105826 | 0.5 | 1.90 | 1.18 | 1.11 | 0.23 | 0.12 | 1.01 | 1.48 | 0.11 | 0.05 |
| | (neutral amino acid | | | | | | | | | | | |
| | transporter), member 5 | | | | | | | | | | | |
| 183 | solute carrier family 12 | U30246 | 1 | 0.55 | 0.38 | 0.36 | 0.18 | 0.18 | 0.76 | 1.11 | 0.31 | 0.31 |
| | (sodium/potassium/chlori | | | | | | | | | | | |
| | de transporters), member | | | | | | | | | | | |
| | 2 | | | | | | | | | | | |
| 184 | solute carrier family 29 | AI342303 | 0 | 0.75 | 0.44 | 0.41 | 0.26 | 0.00 | 0.51 | 0.75 | 0.00 | 0.00 |
| | (nucleoside | | | | | | | | | | | |
| | transporters), member 2 | | | | | | | | | | | |
| 185 | solute carrier family 7 | M80244 | 0.5 | 4.89 | 5.26 | 4.95 | 0.01 | 0.00 | 4.01 | 5.87 | 0.08 | 0.04 |
| | (cationic amino acid | | | | | | | | | | | |
| | transporter, y + system), | | | | | | | | | | | |
| | member 5 | | | | | | | | | | | |
| 186 | sorbitol dehydrogenase | U07361 | 0.5 | 1.31 | 0.96 | 0.90 | 0.16 | 0.08 | 0.80 | 1.18 | 0.05 | 0.02 |
| 187 | SRC-1 | U40396 | 1 | 1.11 | 1.10 | 1.03 | 0.03 | 0.03 | 0.69 | 1.01 | 0.04 | 0.04 |
| 188 | stanniocalcin 2 | AB012664 | 0.5 | 4.77 | 1.21 | 1.14 | 0.62 | 0.31 | 0.99 | 1.45 | 0.52 | 0.26 |
| 189 | stearoyl-CoA desaturase | AA143530 | 0.5 | 0.89 | 0.83 | 0.78 | 0.06 | 0.03 | 0.64 | 0.93 | 0.02 | 0.01 |
| | (delta-9-desaturase) | | | | | | | | | | | |
| 190 | synaptogyrin 2 | AJ002308 | 1 | 0.45 | 0.50 | 0.47 | 0.02 | 0.02 | 0.44 | 0.65 | 0.16 | 0.16 |
| 191 | TATA box binding protein | U21858 | 1 | 1.58 | 1.56 | 1.46 | 0.03 | 0.03 | 0.64 | 0.94 | 0.23 | 0.23 |
| | (TBP)-associated factor, | | | | | | | | | | | |
| | RNA polymerase II, G, | | | | | | | | | | | |
| | 32 kD | | | | | | | | | | | |
| 192 | TGFB inducible early | AF050110 | 1 | 2.07 | 2.23 | 2.09 | 0.00 | 0.00 | 0.51 | 0.75 | 0.44 | 0.44 |
| | growth response | | | | | | | | | | | |
| 193 | transcobalamin I | J05068 | 0.5 | 1.06 | 1.20 | 1.13 | 0.03 | 0.01 | 0.85 | 1.25 | 0.07 | 0.04 |
| | (vitamin B12 binding | | | | | | | | | | | |
| | protein, R binder | | | | | | | | | | | |
| | family) | | | | | | | | | | | |
| 194 | trefoil factor 1 (breast | X05030 | 1.5 | 1.94 | 2.86 | 2.69 | 0.14 | 0.21 | 0.47 | 0.69 | 0.45 | 0.68 |
| | cancer, estrogen- | | | | | | | | | | | |
| | inducible sequence | | | | | | | | | | | |
| | expressed in) | | | | | | | | | | | |
| 195 | tumor necrosis factor | AA994925 | 0 | 0.60 | 0.84 | 0.79 | 0.12 | 0.00 | 0.47 | 0.68 | 0.06 | 0.00 |
| | receptor superfamily, | | | | | | | | | | | |
| | member 7 | | | | | | | | | | | |
| 196 | tumor protein D52-like 1 | U44427 | 1.2 | 3.07 | 4.04 | 3.80 | 0.09 | 0.11 | 0.89 | 1.30 | 0.37 | 0.45 |
| 197 | tumor rejection antigen | X15187 | 1.2 | 2.60 | 2.77 | 2.61 | 0.00 | 0.00 | 0.97 | 1.42 | 0.26 | 0.32 |
| | (gp96) 1 | | | | | | | | | | | |
| 198 | tyrosyl-tRNA synthetase | U89436 | 0.5 | 2.36 | 2.48 | 2.33 | 0.01 | 0.00 | 0.79 | 1.15 | 0.31 | 0.16 |
| 199 | U5 snRNP-specific | D21163 | 0.5 | 0.93 | 0.88 | 0.83 | 0.05 | 0.02 | 0.66 | 0.96 | 0.01 | 0.01 |
| | protein, 116 kD | | | | | | | | | | | |
| 200 | UDP-Gal: betaGlcNAc beta | X13223 | 1 | 1.68 | 2.38 | 2.24 | 0.12 | 0.12 | 0.71 | 1.04 | 0.21 | 0.21 |
| | 1,4- | | | | | | | | | | | |
| | galactosyltransferase, | | | | | | | | | | | |
| | polypeptide 1 | | | | | | | | | | | |
| 201 | unc-51 (C. elegans)-like | AB018265 | 0.5 | 1.74 | 2.09 | 1.97 | 0.05 | 0.03 | 0.73 | 1.06 | 0.21 | 0.11 |
| | kinase 1 | | | | | | | | | | | |
| 202 | v-jun avian sarcoma | AI598150 | 1.2 | 1.28 | 1.56 | 1.47 | 0.06 | 0.07 | 0.84 | 1.23 | 0.02 | 0.02 |
| | virus 17 oncogene | | | | | | | | | | | |
| | homolog | | | | | | | | | | | |
| 203 | zinc finger protein 147 | D21205 | 0 | 0.83 | 0.99 | 0.93 | 0.05 | 0.00 | 0.69 | 1.00 | 0.08 | 0.00 |
| | (estrogen-responsive | | | | | | | | | | | |
| | finger protein) | | | | | | | | | | | |

Table 1 indicates the expression level (BDE(Estrogen)) and weighting factor (K) for each of the genes and ESTs as examples of expression levels of genes and ESTs in MCF-7 cells in the presence of 10 nM 17β-estradiol. Furthermore, as examples of expression level changes, the expression level in 10 nM 17β-estradiol treatment (E(example 1)) and the expression level in 10 nM dioxin treatment (E(example 2)), which were determined completely independently, were indicated. When the evaluation value was calculating from these E(example) values, first, En and BDE(Estrogen)n (n=1 to 203) were calculated to correct the total expression level. Then, calculation of their summation value results in

ΣBDE(Estrogen)n=303.41,

Σ(E(example 1)n)=322.74, and

Σ(E(example 2)n)=207.49.

Furthermore, a value (BDE value) obtained by correcting the expression level using the ratio ΣBDE(Estrogen)n/Σ(En) was calculated. Using this value, each of BDE (example 1) and BDE (example 2) were calculated, and the evaluation value was calculated. As a result, for Table 1, the evaluation values were

(evaluation value, example 1)=16.81

(evaluation value, example 2)=46.19.

In addition, when the calculation was performed considering a weighting factor (K), the values were

(evaluation value, example 1, +K)=11.36 and

(evaluation value, example 2, +K)=32.90.

In this way, the evaluation value obtained in 10-nM dioxin treatment was significantly higher than the evaluation value obtained in 10-nM 17β-estradiol treatment. Therefore, these results showed that the effect of chemical substances having estrogen-like activity, such as environmental hormones and such, on cells could be evaluated by the microarray mentioned above.

### EXAMPLE 2

### The Effect of Dioxin

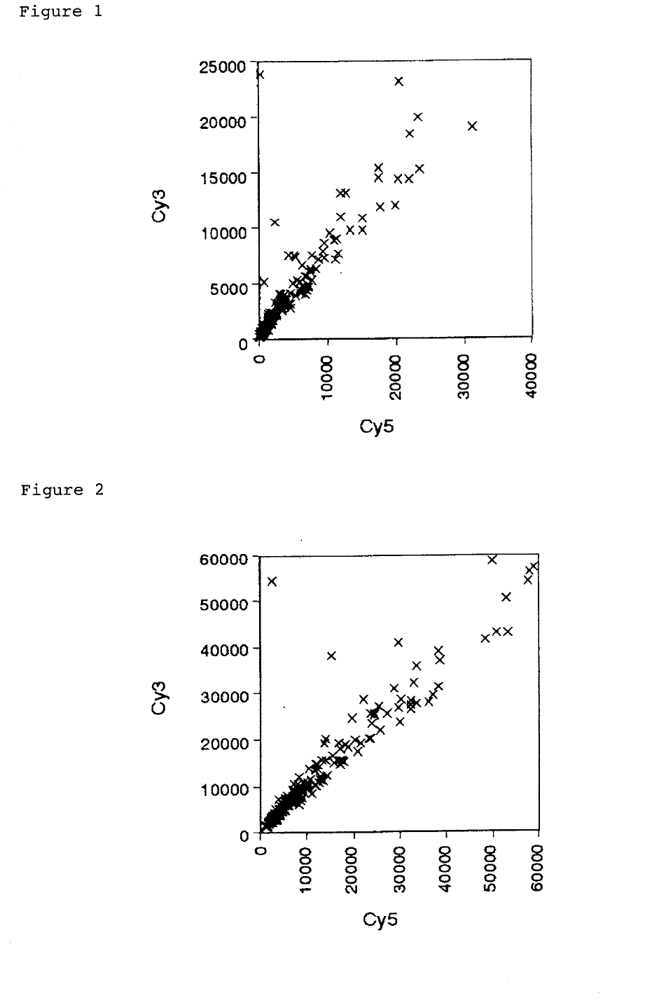

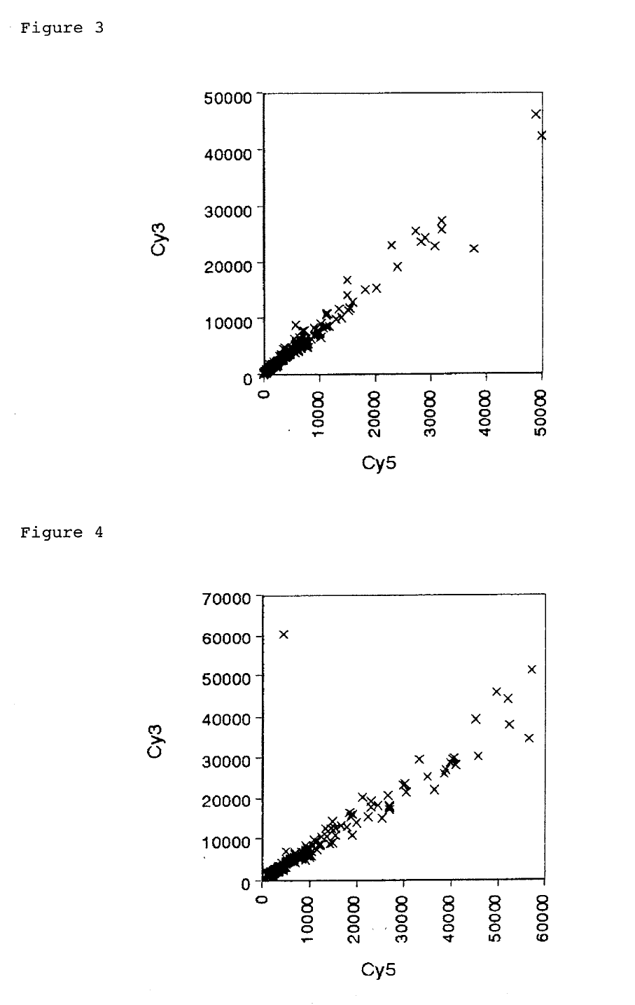

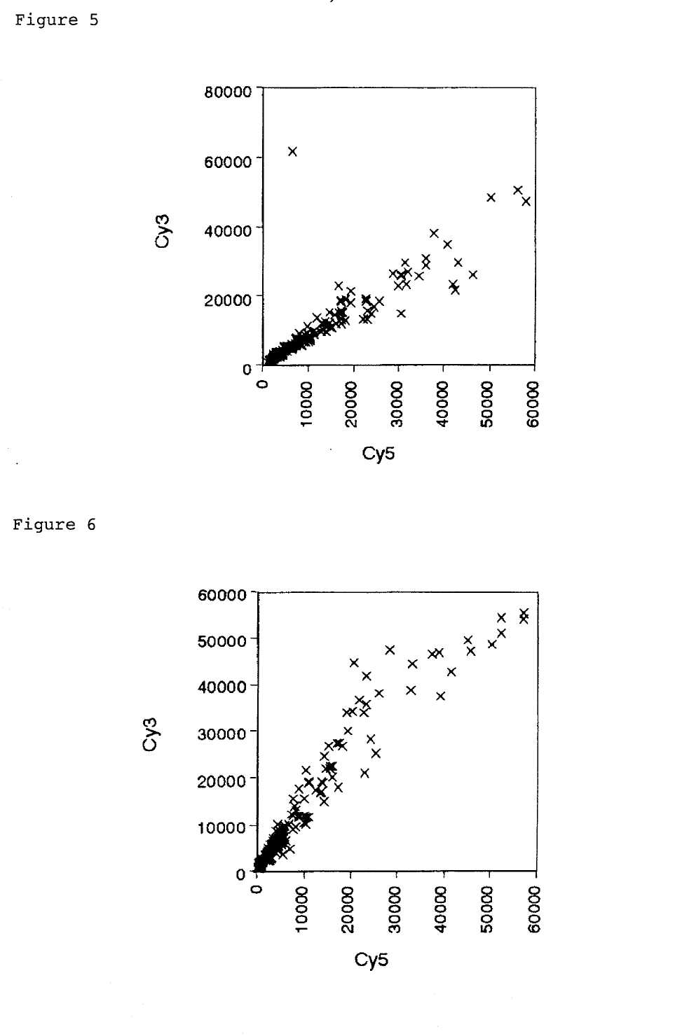

MCF-7 cells or RL95-2 cells were cultivated for 24 hours using 10 nM, 1 nM, or 0.1 nM dioxin (2,3,7,8-tetrachlorodibenzo-p-dioxin, Cambridge Isotope Laboratory (Andover, MA, USA)). After their mRNA was obtained, labeling was performed using fluorescent pigment Cy3 (post-treatment), and estrogen-like activity was evaluated using the microarray of this invention. As a control, after RNA was similarly obtained from the same cells without dioxin treatment, labeling was performed using fluorescent pigment Cy5, and estrogen-like activity was evaluated using the micro array of this invention. The cell culture conditions, obtaining of mRNA, labeling reaction, micro array operation, and such were similar to those in Example 1. These results are indicated in FIGS. 1 to 6.

### EXAMPLE 3

### The Effect of Estrone

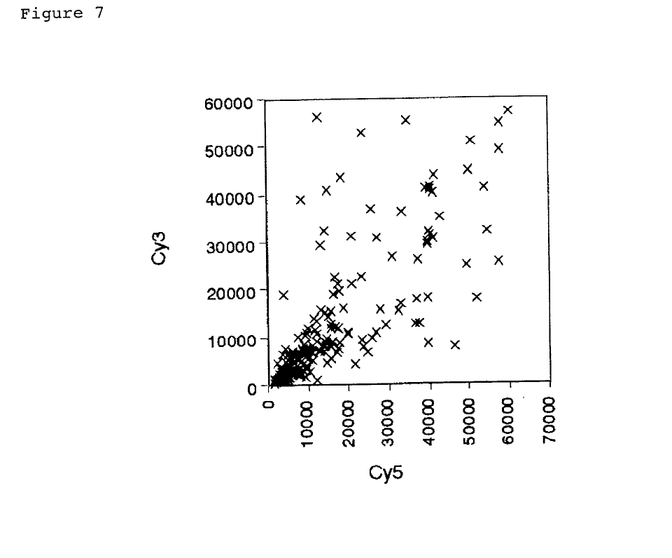

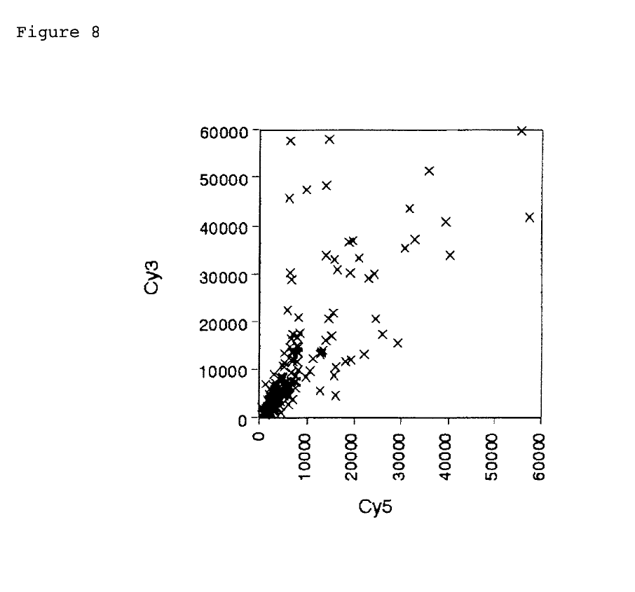

MCF-7 cells were cultivated for 72 hours using 10 nM estrone (Sigma, catalog No. E-9750). After their mRNA was obtained, labeling was performed using fluorescent pigment Cy3 (post-treatment), and estrogen-like activity was evaluated using the microarray of this invention. As a control, after RNA was similarly obtained from the same cells without estrone treatment, labeling was performed using fluorescent pigment Cy5, and estrogen-like activity was evaluated using the microarray of this invention. The cell culture conditions, obtaining of mRNA, labeling reaction, microarray operation, and such were similar to those in Example 1. These results are indicated in FIG. 7.

### EXAMPLE 4

### The Effect of Estriol

MCF-7cells were cultivated for 72 hours using 1 nM estriol (Sigma, catalog No. E-1253). After their mRNA was obtained, labeling was performed using fluorescent pigment Cy3 (post-treatment), and estrogen-like activity was evaluated using the microarray of this invention. As a control, after RNA was similarly obtained from the same cells without estriol treatment, labeling was performed using fluorescent pigment Cy5, and estrogen-like activity was evaluated using the microarray of this invention The cell culture conditions, obtaining of mRNA, labeling reaction, microarray operation, and such were similar to those in Example 1. These results are indicated in FIG. 8.

### EXAMPLE 5

### The Effect of Genistein

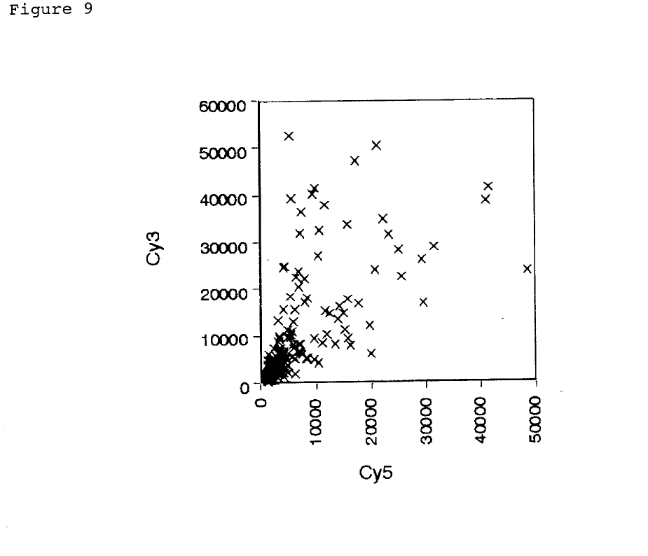

MCF-7 cells were cultivated for 72 hours using 10 nM genistein (Sigma, catalog No. G-6649). After their mRNA was obtained, labeling was performed using fluorescent pigment Cy3 (post-treatment), and estrogen-like activity was evaluated using the microarray of this invention. As a control, after RNA was similarly obtained from the same cells without genistein treatment, labeling was performed using fluorescent pigment Cy5, and estrogen-like activity was evaluated using the microarray of this invention. The cell culture conditions, obtaining of mRNA, labeling reaction, microarray operation, and such were similar to those in Example 1. These results are indicated in FIG. 9.

### EXAMPLE 6

### The Effect of Nonylphenol

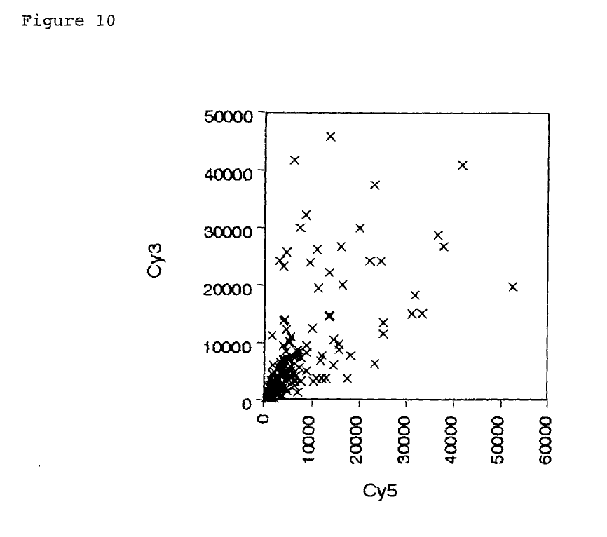

MCF-7 cells were cultivated for 72 hours using 10 nM nonylphenol (Aldrich, catalog No. 29085-8). After their mRNA was obtained, labeling was performed using fluorescent pigment Cy3 (post-treatment), and estrogen-like activity was evaluated using the microarray of this invention. As a control, after RNA was similarly obtained from the same cells without nonylphenol treatment, labeling was performed using fluorescent pigment Cy5, and estrogen-like activity was evaluated using the microarray of this invention. The cell culture conditions, obtaining of mRNA, labeling reaction, microarray operation, and such were similar to those in Example 1. These results are indicated in FIG. 10.

### EXAMPLE 7

### The Effect of Bisphenol A

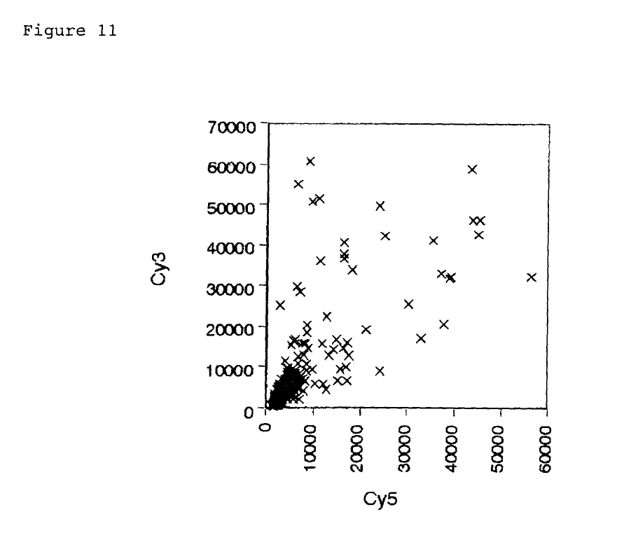

MCF-7 cells were cultivated for 72 hours using 10 nM bisphenol A (Sigma, catalog No. I-0635). After their mRNA was obtained, labeling was performed using fluorescent pigment Cy3 (post-treatment), and estrogen-like activity was evaluated using the microarray of this invention. As a control, after RNA was similarly obtained from the same cells without bisphenol A treatment, labeling was performed using fluorescent pigment Cy5, and estrogen-like activity was evaluated using the microarray of this invention. The cell culture conditions, obtaining of mRNA, labeling reaction, microarray operation, and such were similar to those in Example 1. These results are indicated in FIG. 11.

### EXAMPLE 8

### The Effect of Diethylstilbestrol

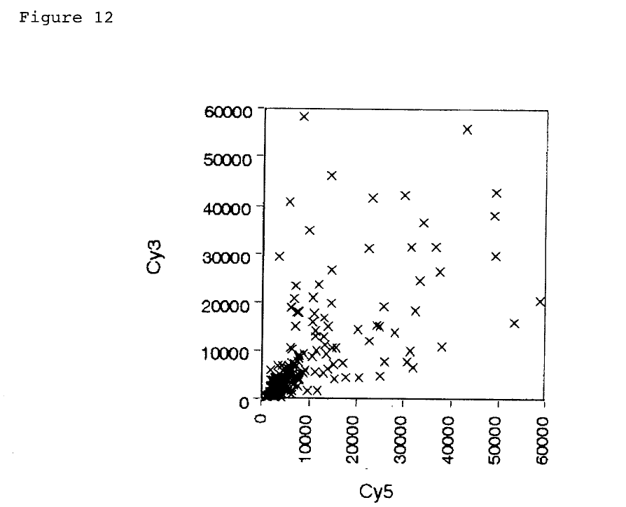

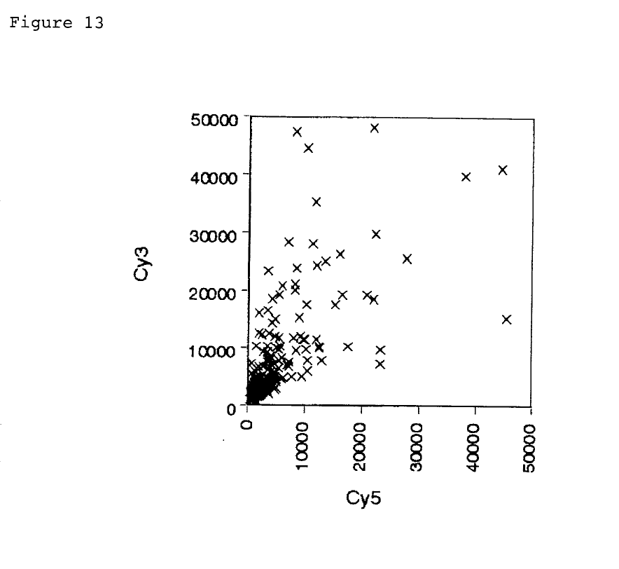

MCF-7 cells were cultivated for 72 hours using 10 nM diethylstilbestrol (DES, Sigma, catalog No. D-4628). After their mRNA was obtained, labeling was performed using fluorescent pigment Cy3 (post-treatment), and estrogen-like activity was evaluated using the microarray of this invention. As a control, after RNA was similarly obtained from the same cells without diethylstilbestrol treatment, labeling was performed using fluorescent pigment Cy5, and estrogen-like activity was evaluated using the microarray of this invention. The cell culture conditions, obtaining of mRNA, labeling reaction, microarray operation, and such were similar to those in Example 1. These results are indicated in FIG. 12.

### EXAMPLE 9

### Control Experiment

As control data for Examples 2 to 8, MCF-7 cells were cultivated for 72 hours using 10 nM 17β-estradiol. After their mRNA was obtained, labeling was performed using fluorescent pigment Cy3 (post-treatment), and estrogen-like activity was evaluated using the microarray of this invention. As a control, after RNA was similarly obtained from the same cells without 17β-estradiol treatment, labeling was performed using fluorescent pigment Cy5, and estrogen-like activity was evaluated using the microarray of this invention. The cell culture conditions, obtaining of mRNA, labeling reaction, microarray operation, and such were similar to those in Example 1. These results are indicated in FIG. 13.

In addition, measurement data corresponding to FIGS. 1 to 13 are indicated in Table 2.

TABLE 2

| Data 1:Cy5 | Data 1:Cy3 | Data 2:Cy5 | Data 2:Cy3 | Data 3:Cy5 | Data 3:Cy3 | Data 4:Cy5 | Data 4:Cy3 | Data 5:Cy5 | Data 5:Cy3 |
|---|---|---|---|---|---|---|---|---|---|
| 284.575 | 165.965 | 3284.93 | 4305.02 | 7650.74 | 4804.385 | 4093.675 | 4111.615 | 5192.145 | 5285.635 |
| 17571.14 | 14535.555 | 38580.355 | 36838.565 | 16025.425 | 12625.265 | 6885.115 | 6178.475 | 7316.775 | 7515.085 |
| 876.685 | 1263.93 | 8585.065 | 9306.34 | 4111.225 | 3252.015 | 2970.325 | 2437.515 | 4100.44 | 3216.08 |
| 3872.395 | 3015.94 | 10159.76 | 10248.94 | 1355.245 | 1264.73 | 11176.86 | 7726.42 | 11346.445 | 8701.92 |
| 9481.28 | 7873.115 | 27327.815 | 25365.005 | 9621.83 | 7748.255 | 22362.57 | 15412.41 | 22669.445 | 18510.405 |
| 19964.365 | 11978 | 59137.13 | 57003.5 | 49951.325 | 42185.48 | 56489.12 | 34359.685 | 58196.675 | 47258.65 |
| 184.33 | 261.47 | 2025.79 | 2578.945 | 2251 | 2180.635 | 2144.14 | 2173.77 | 3758.915 | 3899.565 |
| 382.55 | 485.69 | 2927.18 | 3274.64 | 995.64 | 887.445 | 3173.845 | 2730.92 | 7071.955 | 5971.08 |
| 2519.505 | 2348.735 | 7859.865 | 6612.09 | 3925.795 | 3078.2 | 4882.34 | 3786.35 | 8000.98 | 6554.065 |
| 2981.95 | 2863.83 | 25683.155 | 21807.735 | 11164.88 | 10422.555 | 9235.76 | 7203.065 | 15285.515 | 10805.915 |
| 294.805 | 338.34 | 1492.835 | 1421.38 | 1949.715 | 1464.07 | 1003.36 | 1070.33 | 1984.94 | 1659.16 |
| 4560.2 | 4128.275 | 12529.425 | 10726.62 | 9913.48 | 7544.28 | 3239.92 | 1970.01 | 3736.055 | 2814.215 |
| 413.22 | 500.08 | 3133.745 | 4237.555 | 1529.195 | 1394.395 | 1964.74 | 1836.63 | 3384.485 | 3204.8 |
| 1636.51 | 2044.825 | 4546.385 | 4482.29 | 6297.885 | 4332.25 | 15617.375 | 10883.205 | 11309.615 | 10064.945 |
| 755.38 | 852.875 | 2345.11 | 2852.345 | 1837.515 | 1855.13 | 3917.89 | 3599.54 | 3058.565 | 3509.02 |
| 112.19 | 143.37 | 7202.55 | 9386.91 | 190.435 | 343.42 | 1182.505 | 1055.09 | 5694.01 | 4998.67 |
| 139.65 | 149.63 | 1843.205 | 1855.23 | 955.955 | 930.365 | 1579.265 | 2068.115 | 2290.655 | 1824.9 |
| 17742.045 | 11882.83 | 32283.535 | 27196.295 | 1715.355 | 1496.03 | 25396.05 | 14966.865 | 24046.3 | 14925.205 |
| 1323.89 | 1273.055 | 14245.115 | 15706.825 | 855.435 | 760.595 | 3188.37 | 2580.445 | 8142.14 | 6896.215 |
| 1434.385 | 1316.26 | 4173.49 | 4047.87 | 1 | 1 | 4495.17 | 3669.35 | 3456.47 | 2683.48 |
| 5422.815 | 3848.475 | 12983.315 | 11349.87 | 6495.73 | 5331.07 | 15050.675 | 8948.19 | 15137.075 | 10823.34 |
| 561.505 | 543.37 | 3304.63 | 3938.93 | 3921.265 | 3040.665 | 3973.93 | 3184.79 | 4179.015 | 3414.35 |
| 4262.825 | 3118.95 | 8295.295 | 6149.82 | 702.67 | 1048.93 | 3662.165 | 2531.335 | 3611.3 | 2644.05 |
| 5062.375 | 5027.195 | 16939.475 | 19248.48 | 6558.05 | 6505.725 | 6120.835 | 4768.11 | 9292.645 | 8933.67 |
| 826.955 | 635.365 | 5703.79 | 5650.18 | 6789.78 | 5702.47 | 3341.195 | 2420.985 | 5359.505 | 3927.42 |
| 267.685 | 323.61 | 3889.545 | 4145.185 | 691.06 | 654.94 | 1111.775 | 1181.69 | 1847.93 | 1434.685 |
| 6599.74 | 4634.705 | 23360.12 | 20079.6 | 1187.9 | 1499.885 | 4548.74 | 2956.04 | 9519.98 | 6797.95 |
| 390.865 | 23863.995 | 2840.74 | 54167.325 | 204.63 | 366.555 | 4419.75 | 60121.255 | 6694.755 | 61695.52 |
| 1958.23 | 1798.825 | 5030.05 | 5731.945 | 4368.34 | 3414.82 | 5363.895 | 4748.5 | 5529.99 | 5444.54 |
| 476.22 | 457.655 | 5281.385 | 7620.92 | 9839.315 | 7365.01 | 1912.195 | 1777.555 | 2415.32 | 3203.115 |
| 6497.475 | 4694.995 | 17106.515 | 15717.66 | 2421.22 | 2032.99 | 17816.48 | 13084.975 | 17382.15 | 15500.125 |
| 10338.125 | 9458.76 | 25526.67 | 27073.37 | 10228.505 | 8970.005 | 18917.45 | 15536.935 | 9993.25 | 11125.305 |
| 11399.53 | 8963.85 | 23999.57 | 23520.37 | 300.385 | 428.03 | 15434.585 | 12465.875 | 16019.745 | 14579.405 |
| 1026.64 | 977.185 | 3443.72 | 3179.965 | 330.325 | 347.64 | 3032.105 | 2384.87 | 6584.525 | 4903.205 |
| 6771.91 | 4014.65 | 17584.365 | 15688.285 | 7058.215 | 5251.425 | 2611.98 | 1670.05 | 3533.275 | 3019.53 |
| 80.455 | 319.685 | 2541.69 | 2784.45 | 1159.05 | 1116.23 | 1917.155 | 1534.2 | 4283.325 | 3166.685 |
| 170.35 | 449.565 | 1328.575 | 1364.655 | 832.81 | 888.33 | 2562.805 | 2259.47 | 3727.1 | 2984.7 |
| 305.005 | 416.13 | 1382.175 | 1314.095 | 628.39 | 793.72 | 36513.3 | 21957.78 | 30011 | 22906.33 |
| 1329.18 | 2301.6 | 4013.685 | 7214.85 | 961.945 | 1360.65 | 1807.165 | 2098.125 | 2518.69 | 2691.035 |
| 1792.2 | 1370.53 | 6995.655 | 7865.34 | 8489.81 | 6327.895 | 4765.14 | 3089.11 | 6583.77 | 6105.61 |
| 3319.8 | 3494.725 | 10586.1 | 13746.77 | 6007.685 | 5027.195 | 18417.685 | 16304.835 | 19457.165 | 21051.79 |
| 1276.945 | 1096.025 | 8770.16 | 9436.045 | 4956.57 | 4599.015 | 8675.71 | 6090.63 | 16253.18 | 11824.92 |
| 1178.615 | 1209.18 | 8892.58 | 9675.05 | 10257.215 | 6610.875 | 4741.39 | 3040.11 | 7622.81 | 6132.99 |
| 407.17 | 466.135 | 4701.205 | 5402.715 | 6192.205 | 5281.25 | 2563.65 | 1882.075 | 3901.21 | 3786.41 |
| 1788.245 | 1204.47 | 13562.55 | 11119.71 | 31979.015 | 27226.39 | 9218.005 | 5038.81 | 17387.31 | 13202.91 |
| 182.545 | 483.81 | 1791.345 | 1964.41 | 1182.85 | 1405.73 | 13409.71 | 12536.935 | 8056.195 | 9075.925 |
| 1265.71 | 1226.27 | 7899.15 | 9482.61 | 5519.89 | 4480.51 | 9139.935 | 7128 | 9971.58 | 9030.055 |
| 4193.435 | 3117.3 | 18305.275 | 18918.035 | 1310.875 | 1147.265 | 14395.24 | 8824.505 | 13782.545 | 11188.93 |
| 405.83 | 463 | 5760.325 | 6492.78 | 9236.875 | 6978.325 | 2494.21 | 2230.035 | 4927.62 | 5386.395 |
| 1108.955 | 1606.37 | 4100.365 | 5041.84 | 3521.89 | 3170.035 | 1112.72 | 1047.52 | 2937.735 | 2652.485 |
| 5593.395 | 5290.985 | 30265.31 | 28402.515 | 12833.415 | 9786.43 | 13778.845 | 10665.28 | 17415.03 | 11856.43 |
| 5254.79 | 7498.83 | 14171.195 | 20088.015 | 3428.71 | 4403.97 | 14608.325 | 12497.13 | 14634.75 | 15003.525 |
| 17507.88 | 15341.035 | 51013.07 | 43048.485 | 28895.595 | 24333.32 | 49683.375 | 45753.275 | 31550.295 | 29439.635 |
| 1513.435 | 2377.095 | 2515.39 | 3278.53 | 1147.47 | 1190.49 | 3628.935 | 2710.72 | 3841.38 | 3244.45 |
| 2947.265 | 4097.84 | 11612.69 | 13403.54 | 14024.275 | 10021.51 | 9757.125 | 7182.86 | 14078.42 | 11294.16 |
| 6072.645 | 5186.68 | 23754.78 | 20035 | 11622.55 | 8381.68 | 2539.365 | 2564.01 | 2838.92 | 1957.93 |
| 3837.485 | 3531.655 | 10765.575 | 11375.055 | 1043.335 | 1840.83 | 2197.02 | 1852.615 | 2987.525 | 2418.595 |
| 22285.35 | 18351 | 38267.29 | 39060.405 | 3748.745 | 3007.4 | 30249.335 | 23392.51 | 17367.985 | 18520.485 |
| 8457.15 | 6269.035 | 32519.25 | 26390.775 | 37636.525 | 22204.485 | 35002.74 | 25070.43 | 42476.52 | 21757.205 |
| 20415.835 | 14377.26 | 33740.59 | 27659.245 | 28194.385 | 23517.525 | 57324.615 | 50994.67 | 46595.385 | 26105.04 |
| 475.09 | 610.3 | 3872.94 | 4313.6 | 5430.355 | 3965.46 | 1602.465 | 1348.9 | 3801.905 | 3643.455 |
| 1119.065 | 1168.635 | 5810.9 | 8071.82 | 999.175 | 1186.98 | 4998.675 | 7029.09 | 6209.055 | 6403.635 |
| 2065.715 | 2354.345 | 7017.545 | 6162.38 | 6152.91 | 3992.5 | 7743.625 | 5430.155 | 14810.025 | 12166.77 |
| 23398.265 | 19818.42 | 33085.285 | 31953.65 | 11197.625 | 10670.185 | 40988.06 | 27870.37 | 28851.965 | 26522.35 |
| 366.17 | 584.38 | 1798.565 | 2278.79 | 723.985 | 871.405 | 1942.05 | 2001.915 | 1541.775 | 1174.92 |
| 23655.63 | 15263.78 | 53110.74 | 50326.095 | 18072.7 | 15027.23 | 26789.87 | 17042.14 | 17720.96 | 15180.715 |
| 7702.42 | 7478.56 | 23556.03 | 25419.725 | 8994.375 | 6774.24 | 6615.93 | 4832.795 | 12891.905 | 9659.515 |
| 10963.01 | 8889.295 | 33586.74 | 35735.97 | 7058.095 | 7806.875 | 40413.235 | 29324.73 | 40932.04 | 34779.51 |
| 972.21 | 780.89 | 4298.055 | 4053.655 | 10659.81 | 8139.045 | 4349.835 | 2517.935 | 4337.975 | 3367.66 |
| 379.55 | 436.55 | 1764.695 | 1728.21 | 174.24 | 560.245 | 19271.065 | 16141.255 | 16912.99 | 15265.275 |
| 1462.51 | 1667.03 | 3112.84 | 3726.025 | 275.22 | 305.83 | 4347.875 | 3512.055 | 6336.17 | 5740.485 |
| 258.015 | 352.76 | 3822.285 | 4599.48 | 5230.275 | 4200.36 | 1487.59 | 1554.56 | 3429.61 | 3608.925 |
| 350.67 | 434.64 | 2149.335 | 2173.655 | 2833.41 | 2242.2 | 1416.72 | 1348.075 | 2788.98 | 2125.685 |
| 353.54 | 391.915 | 1456.645 | 1332.93 | 1526.94 | 1492.365 | 2084.55 | 1653.3 | 3026.3 | 2668.035 |
| 2388.315 | 10444.085 | 2135.855 | 2010.23 | 5786.895 | 8795.92 | 2025.75 | 1316.005 | 1830.175 | 1348.215 |
| 617.3 | 620.88 | 3782.585 | 5006.115 | 5660.2 | 5065.17 | 1933.725 | 1918.7 | 3416.845 | 3538.98 |
| 265.605 | 286.47 | 5830.68 | 6919.415 | 2352.275 | 2498.28 | 2281.815 | 2174.83 | 3928.03 | 2465.075 |
| 2562.3 | 2290.75 | 6128.585 | 5830.125 | 2162.42 | 1886.565 | 4401.3 | 3197.625 | 3533.06 | 2914.47 |
| 1725.595 | 1406.55 | 3552.43 | 3050.865 | 1026.45 | 1107.315 | 3653.085 | 3008.22 | 3140.94 | 2516.285 |
| 698.835 | 1165.625 | 1752.56 | 1892.185 | 684.105 | 969.81 | 2729.355 | 2672.955 | 3378.885 | 2373.685 |
| 2012.465 | 1682.855 | 3906.435 | 3541.315 | 2631.235 | 2187.625 | 5001.44 | 3808.96 | 6195.04 | 5212.61 |
| 3379.165 | 2560.05 | 18118.005 | 15206.375 | 15194.345 | 11275.005 | 11950.43 | 8571.855 | 23014.52 | 15548.81 |
| 1415.67 | 1507.64 | 3002.645 | 3432.01 | 2544.405 | 2219.945 | 3862.54 | 3466.555 | 3115.06 | 3304.805 |
| 2749.22 | 2834.92 | 7565.7 | 7739.105 | 771.13 | 697.065 | 16683.075 | 13454.475 | 17460.08 | 15378.29 |
| 662.155 | 669.24 | 3276.46 | 3181.035 | 3555.845 | 2823.075 | 1899.345 | 1577.415 | 1819.24 | 1537.16 |
| 1914.855 | 1990.94 | 6962.665 | 9242.575 | 1 | 208.97 | 4283.16 | 3590.315 | 3794.785 | 3773.25 |
| 2563.385 | 2227.845 | 7225.55 | 6569.165 | 2418.14 | 1693.14 | 6671.855 | 4449.8 | 6706.33 | 5031.12 |
| 1691.77 | 1578.32 | 9433.215 | 8801.29 | 5870.355 | 4794.665 | 5154.52 | 4073.675 | 6434.21 | 5487.96 |
| 4360.105 | 7497.55 | 13837.06 | 19154.09 | 11290.765 | 8459.9 | 26616.27 | 20492.985 | 32207.715 | 26651.665 |
| 3822.36 | 4047.565 | 10076.84 | 10869.04 | 11393.12 | 10767.325 | 3120.855 | 2122.495 | 7683.705 | 6390.32 |
| 1917.555 | 1596.925 | 8139.18 | 7843.65 | 2369.045 | 2234.245 | 4126.63 | 3130.64 | 6599.06 | 4332.29 |
| 3027.755 | 3970.615 | 13245.225 | 15579.595 | 357.425 | 462.005 | 1260.535 | 1755.615 | 2752.525 | 2075.335 |
| 283.36 | 418.755 | 3306.645 | 3675.72 | 1 | 1 | 1864.38 | 1804.745 | 3165.36 | 2266.675 |
| 12890.93 | 13078.115 | 24484.38 | 25543.665 | 27298.625 | 25458.455 | 40575.435 | 29669.355 | 36012.745 | 30986.055 |
| 269.185 | 410.815 | 3018.965 | 3240.695 | 828.05 | 655.46 | 1351.93 | 1076.11 | 2148.02 | 1761.71 |
| 1436.55 | 1334.26 | 3581.455 | 2817.685 | 1173.64 | 1079.62 | 3212.83 | 2767.75 | 5762.875 | 5510.355 |
| 267.47 | 344.335 | 3161.615 | 3967.935 | 4610.935 | 3606.585 | 2187.365 | 1647.075 | 3606.24 | 2983.93 |
| 152.89 | 565.725 | 3358.46 | 3531.935 | 1655.935 | 1682.345 | 1863.54 | 1697.535 | 3188.78 | 2667.17 |
| 1776.395 | 1692.35 | 8031.94 | 10609.36 | 5928.135 | 5749.93 | 12446.215 | 10554.635 | 11963.675 | 13763.965 |
| 129.72 | 400.775 | 2811.875 | 3556.57 | 937.235 | 1178.47 | 1141.915 | 1228.96 | 3901.805 | 3218.61 |
| 881.005 | 580.865 | 5614.65 | 6341.765 | 1377.02 | 1312.46 | 10015.46 | 5599.68 | 23019.085 | 13182.415 |
| 120.025 | 206.05 | 2322.9 | 2399.27 | 1672.76 | 1374.63 | 1840.76 | 1843.595 | 2057.095 | 1855.18 |
| 197.105 | 348.15 | 1786.59 | 1910.765 | 1805.455 | 1553.99 | 2178.525 | 1792.72 | 3671.715 | 3614.44 |
| 7077.145 | 5669.855 | 30085.92 | 23841.79 | 23893.67 | 19054.775 | 30504.135 | 21267.37 | 22254.485 | 13279.355 |
| 1069.475 | 748.08 | 4249.08 | 3634.03 | 2021.225 | 1740.855 | 1819.935 | 1677.81 | 2538.065 | 2240.74 |
| 3868.155 | 3189.77 | 5414.07 | 4873.89 | 4040.785 | 3481.125 | 9292.235 | 8379.185 | 6881.575 | 5579.74 |
| 1084.935 | 1234.935 | 3444.38 | 3753.325 | 1421.78 | 1598.2 | 4079.99 | 3205.045 | 3451.935 | 3715.03 |
| 175.615 | 405.235 | 4078.47 | 5197.715 | 403.89 | 750.415 | 2801.35 | 2755.155 | 2471.925 | 1770.685 |
| 1226.445 | 1944.03 | 3303.375 | 5021.995 | 1861.09 | 1695.28 | 1509.71 | 1187.245 | 2074.235 | 1792.21 |
| 256.94 | 395.125 | 2099.57 | 2314.015 | 690.485 | 989.4 | 1728.45 | 1610.635 | 2888.975 | 2360.2 |
| 3227.365 | 2873.095 | 15835.15 | 15086.835 | 2292.335 | 1956.975 | 5108.76 | 3285.045 | 8214.58 | 5880.565 |
| 388.23 | 497.43 | 2344.005 | 2300.915 | 629.345 | 928.55 | 1915.355 | 1835.25 | 2420.62 | 1977.495 |
| 343.365 | 490.945 | 2698.13 | 3150.555 | 3145.54 | 2734.37 | 2014.29 | 1793.025 | 2271.5 | 2084.64 |
| 682.95 | 670.53 | 4891.355 | 4357.61 | 5663.965 | 5397.985 | 2803.625 | 1983.585 | 9110.34 | 6748.15 |
| 2267.53 | 2099.295 | 9118.915 | 9006.145 | 1172.605 | 1082.59 | 1817.665 | 1536.985 | 2289.92 | 1798.955 |
| 2009.165 | 2112.865 | 8886.82 | 9392.465 | 568.265 | 685.75 | 3687.305 | 2971.315 | 3652.995 | 2804.69 |
| 1342.77 | 1301.63 | 4414.315 | 4419.26 | 2140.93 | 1543.595 | 3364.725 | 2975.985 | 3473.625 | 2678.02 |
| 781.125 | 822.12 | 2922.605 | 3084.94 | 1627.13 | 2135.755 | 2917.535 | 2420.105 | 2988.355 | 2413.195 |
| 1980.475 | 2265 | 7330.8 | 8563.165 | 1356.33 | 1200.175 | 6772.24 | 6387.44 | 8617.54 | 8534.645 |
| 382.365 | 414.18 | 2796.045 | 2901.26 | 233.645 | 317.725 | 2248.12 | 2001.435 | 2677.415 | 2170.475 |
| 11957.45 | 13150.35 | 22348.68 | 28505.975 | 8895.34 | 8253.775 | 5337.36 | 4350.665 | 8622.895 | 7954.09 |
| 1978.205 | 1735.305 | 4850.325 | 6326.94 | 3147.6 | 2468.605 | 5776.78 | 4669.55 | 4944.82 | 5541.25 |
| 1818.435 | 1300.48 | 17158.925 | 14630.93 | 8061.135 | 5603.405 | 2426.755 | 1944.525 | 8762.135 | 5641.88 |
| 820.39 | 851.765 | 1985.915 | 2173.455 | 224.75 | 343.935 | 6024.625 | 5407.705 | 7098.755 | 6247.76 |
| 7690.045 | 5998.245 | 11720.63 | 10158.035 | 3878.415 | 3328.01 | 7045.98 | 4341.89 | 8175.3 | 6191.68 |
| 407.105 | 494.055 | 2435.37 | 2635.405 | 336.495 | 358.995 | 2093.25 | 1935.98 | 1987.66 | 1668.835 |
| 725.675 | 1015.7 | 2216.19 | 2589.8 | 668.91 | 857.785 | 2370.855 | 2320.75 | 2248.12 | 2100.285 |
| 509.63 | 650.27 | 1803.24 | 2018.765 | 1531.585 | 1360.25 | 1536.37 | 1583.28 | 1724.52 | 1342.925 |
| 1741.075 | 2051.735 | 7302.66 | 8570.795 | 4634.645 | 3680.365 | 10777.315 | 8543.715 | 13906.045 | 12479.185 |
| 3061.15 | 2979.225 | 10786.495 | 11430.225 | 3815.185 | 3091.395 | 8360.545 | 6937.475 | 8056.63 | 7409.54 |
| 1001 | 1027.99 | 4369.325 | 5571.735 | 4693.245 | 4364.87 | 3632.2 | 3351.74 | 4602.25 | 4593.425 |
| 505.9 | 530.155 | 2305.34 | 2445.885 | 1099.21 | 1104.83 | 3399.725 | 1930.285 | 4450.78 | 3276.885 |
| 740.25 | 584.375 | 8567.655 | 6991.205 | 7860.055 | 4813.345 | 4312.42 | 2614.25 | 4540.825 | 2947.68 |
| 1273.865 | 1015.91 | 3160.2 | 2947.135 | 291.49 | 280.865 | 2051.22 | 1668.1 | 2232.41 | 1639.225 |
| 6063.925 | 4534.495 | 21681.46 | 19175.03 | 2797.955 | 3262.705 | 1780.89 | 1708.57 | 2581.055 | 1808.985 |
| 775.96 | 645.17 | 2642.13 | 2202.26 | 3504.94 | 2564.45 | 1726.225 | 1154.78 | 2962.54 | 2477.425 |
| 9665.205 | 7194.645 | 38432.225 | 31270.91 | 31888.685 | 25855.47 | 39952.41 | 28630.4 | 41882.28 | 23390.665 |
| 7828.95 | 5220.715 | 58095.2 | 56197.845 | 13429.235 | 11516.24 | 24637.74 | 18359.485 | 35959.415 | 28602.185 |
| 31338.685 | 18945.685 | 53487.62 | 42953.055 | 48842.22 | 46114.32 | 45727.915 | 30070.705 | 31822.03 | 23099.395 |
| 15231.92 | 9735.485 | 32463.445 | 28139.27 | 166.145 | 231.875 | 10547.395 | 7065.675 | 9596.055 | 6721.47 |
| 2183.67 | 2214.14 | 8264.555 | 12134.22 | 1138.61 | 941.6 | 6894.055 | 5471.405 | 4941.08 | 3858.845 |
| 367.835 | 376.645 | 4365.175 | 5126.64 | 1593.205 | 1255.87 | 3762.385 | 2982.46 | 7210.105 | 5469.725 |
| 4020.095 | 3718.52 | 7855.61 | 7479 | 1475.92 | 1337.745 | 5903.335 | 4561.11 | 5960.825 | 4911.51 |
| 506.385 | 553.455 | 2966.345 | 2669.995 | 736.89 | 668.035 | 1303.91 | 1332.52 | 3269.435 | 2697.615 |
| 2622.675 | 2149.295 | 8660.865 | 9002.08 | 5980.265 | 5329.565 | 8770.455 | 6001.12 | 10164.35 | 8406.815 |
| 6488.26 | 6683.825 | 8094.67 | 9549.68 | 6293.145 | 4965.135 | 22928.15 | 19309.455 | 17089.31 | 18429.9 |
| 270.37 | 309.475 | 1902.635 | 1753.03 | 2411.17 | 1820.87 | 2191.975 | 1496.18 | 3120.06 | 2721.49 |
| 73.115 | 399.145 | 4249.215 | 4871.325 | 3275.55 | 3401.165 | 1103.69 | 1259.76 | 1548.165 | 1405.915 |
| 67.14 | 159.755 | 1689.55 | 1768.65 | 2072.11 | 1181.855 | 1277.35 | 1175.395 | 1770.97 | 1351.31 |
| 1133.215 | 1358.005 | 2588.63 | 3571.77 | 193.855 | 272.56 | 1364.085 | 1405.705 | 1868.275 | 1564.675 |
| 1440.455 | 740 | 5998.715 | 5893.73 | 5873.175 | 4716.38 | 1035.185 | 1113.29 | 3151.045 | 3547.285 |
| 11240.775 | 7126.34 | 36182.805 | 27848.735 | 4268.195 | 3137.39 | 52347.625 | 37843.955 | 43144.68 | 29681.495 |
| 9546.37 | 8581.82 | 20301.445 | 19819.52 | 7700.555 | 5371.745 | 27026.08 | 18309.32 | 30689.875 | 25993.24 |
| 116.565 | 137.715 | 3652.155 | 4327.715 | 7460.805 | 5030.41 | 1338.04 | 1250.025 | 2843.99 | 2745.395 |
| 117.09 | 306.085 | 2072.47 | 2149.27 | 689 | 636.155 | 1474.285 | 1483.97 | 2112.345 | 1535.38 |
| 22068.295 | 14420.655 | 57764.59 | 54105.13 | 3250.04 | 3056.915 | 38816.25 | 26958.985 | 34560.805 | 25753.11 |
| 465.365 | 500.335 | 6015.235 | 6495.225 | 5112.02 | 4420.945 | 2017.985 | 1695.815 | 2837.985 | 2382.505 |
| 1472.02 | 1704.165 | 6070.66 | 7229.035 | 9788.6 | 7105.705 | 2642.52 | 2260.655 | 3885.12 | 4096.455 |
| 20648.24 | 23101.08 | 49953.645 | 58491.985 | 22926.68 | 22944.4 | 45078.93 | 39283.34 | 37814.075 | 37856.655 |
| 3724.84 | 2932.97 | 5036.92 | 4774.605 | 652.115 | 684.95 | 9424.65 | 7620.395 | 4517.665 | 3782.015 |
| 574.705 | 561.365 | 6789.945 | 8018.72 | 5623.84 | 3863.45 | 6602.615 | 5319.79 | 7610.81 | 7846.365 |
| 3447.625 | 2725.655 | 5896.985 | 5910.19 | 4174.005 | 3371.18 | 11086.84 | 9735.53 | 10518.215 | 8102.555 |
| 155.785 | 346.37 | 3705.815 | 4919.995 | 7540.575 | 5383 | 2593.83 | 2518.37 | 3390.335 | 3720.905 |
| 1354.695 | 1371.45 | 7101.72 | 7122.77 | 3299.365 | 3556.175 | 6736.25 | 5255.11 | 7357.73 | 5709.7 |
| 5333.41 | 7323.215 | 19750.665 | 24477.11 | 14885.27 | 14114.775 | 14840.4 | 14274.515 | 18371.23 | 18986.63 |
| 1360.675 | 1718.31 | 4178.225 | 5054.345 | 2946.225 | 3102.9 | 4895.2 | 3907.165 | 14098.585 | 9465.455 |
| 7190.205 | 4625.14 | 12793.445 | 11858.04 | 1477.95 | 1837.235 | 3954.7 | 3022.82 | 3901.8 | 2900.14 |
| 8706.585 | 7075.675 | 24507.59 | 25233.12 | 1905.005 | 1853.42 | 19934.99 | 14045.215 | 22804.475 | 19038.085 |
| 7624.835 | 6307.59 | 10194.835 | 9849.985 | 6930.85 | 5721.095 | 6052.265 | 4762.195 | 5408.87 | 4148.76 |
| 1380.915 | 1204.18 | 8629.36 | 8211.445 | 858.12 | 865.925 | 3082.835 | 2532.345 | 8419.57 | 5676.25 |
| 3738.15 | 3938.535 | 12096.655 | 14722.77 | 30587.045 | 22709.465 | 11335.195 | 9372.46 | 11223.955 | 9417.83 |
| 11500.215 | 7603.92 | 29559.965 | 26604.845 | 2431.375 | 1515.31 | 19084.865 | 10923.09 | 24739.93 | 16730.12 |
| 400.23 | 455.385 | 5143.655 | 6938.27 | 3962.965 | 3195.77 | 22929.195 | 17916.895 | 19441.81 | 18092.145 |
| 441.535 | 981.5 | 5853.935 | 7230.91 | 5438.725 | 6160.135 | 5347.94 | 4691.495 | 4125.195 | 3530.95 |
| 7170.915 | 4921.055 | 11944.665 | 10323 | 5134.26 | 4735.785 | 9422.54 | 6052.785 | 6508.24 | 4991.025 |
| 15199.27 | 10891.345 | 37219.125 | 29424.665 | 14891.895 | 16750.985 | 38543.295 | 25737.94 | 25782.55 | 18564.86 |
| 830.81 | 918.305 | 1973.3 | 2101.37 | 1183.715 | 1074.875 | 2345.465 | 2020.785 | 2692.145 | 2501.05 |
| 289.13 | 467.285 | 3267.475 | 4290.52 | 4118.1 | 3459.01 | 1408.82 | 1437.275 | 2608.47 | 2451.225 |
| 4511.08 | 2756.735 | 7861.875 | 6507.315 | 1 | 1 | 2167.95 | 1880.045 | 4400.825 | 3417.745 |
| 3908.42 | 3519.765 | 9041.555 | 7585.825 | 2405.71 | 2387.395 | 11580.015 | 7343.625 | 17488.29 | 11884.835 |
| 6761.99 | 5591.27 | 9426.24 | 10676.43 | 5698.73 | 4403.685 | 5049.06 | 4419.96 | 3782.385 | 3248.335 |
| 649.45 | 935.395 | 18851.58 | 18287.91 | 3806.145 | 4830.415 | 2491.425 | 2156.52 | 18381.205 | 12625.54 |
| 3231.76 | 3490.42 | 12175.61 | 14773.51 | 906.03 | 857.25 | 7037.475 | 6673.56 | 7387.91 | 7070.565 |
| 1296.65 | 944.48 | 17206.54 | 17994.32 | 7382.135 | 6328.04 | 8365.14 | 5363.17 | 10098.405 | 6621.74 |
| 897.4 | 5131.715 | 15266.335 | 38151.51 | 15538.55 | 11642.365 | 33241.085 | 29449.775 | 50412.295 | 48488.275 |
| 2664.87 | 3052.73 | 12223.74 | 13811.485 | 1195.08 | 1052.4 | 12266.75 | 8771.185 | 13175.86 | 10284.265 |
| 810.66 | 798.13 | 2818.24 | 2955.045 | 576.4 | 631.3 | 1849.495 | 1787.94 | 3308.955 | 2503.47 |
| 922.385 | 1306.94 | 29550.475 | 40865.845 | 4873.955 | 4698.395 | 2428.155 | 2257.22 | 10511.485 | 7466.52 |
| 12086.625 | 10958.15 | 28827.675 | 30824.42 | 6767.46 | 6221.335 | 52081.54 | 44211.7 | 56390.495 | 50551.48 |
| 6992.84 | 4399.665 | 11054.55 | 8430.115 | 2840.05 | 2506.25 | 10437.835 | 5995.33 | 10576.455 | 7103.615 |
| 3233.02 | 2966.82 | 9706.44 | 9645.57 | 1842.455 | 1886.81 | 6344.035 | 4821.145 | 10459.675 | 8320.57 |
| 13309.78 | 9761.955 | 48747.26 | 41357.08 | 20079.2 | 15236.075 | 26878.25 | 17944.215 | 30655.82 | 25466.42 |
| 560.325 | 681.855 | 1127.03 | 1148.16 | 1099.63 | 1084.565 | 1977.13 | 1562.365 | 1053.49 | 909.38 |
| 4602.155 | 3072.645 | 14432.1 | 12399.945 | 6572.01 | 4498.385 | 4368.465 | 3086.405 | 2310.495 | 1710.755 |
| 6253.38 | 4151.835 | 21134.07 | 17340.84 | 4541.675 | 3416.835 | 30006.395 | 23240.46 | 30650.49 | 14960.445 |
| 3124.735 | 3955.18 | 7155.57 | 10596.265 | 3144.575 | 3355.89 | 7528.64 | 5563.895 | 7809.75 | 6436.295 |
| 2328.235 | 3211.75 | 5924.35 | 7520.92 | 2264.09 | 2233.885 | 21145.325 | 20425.275 | 16940.265 | 22764.27 |
| 2877.015 | 3226.76 | 15670.04 | 16362.45 | 7305.61 | 7635.88 | 10219.04 | 7603.95 | 12763.16 | 11842.385 |
| 748.115 | 698.79 | 3173.05 | 2808.665 | 912.255 | 1044.025 | 2871.685 | 2169.82 | 4612.16 | 3579.855 |
| 3384.64 | 3433.84 | 5179.415 | 5063.74 | 2214.795 | 2260.22 | 12336.45 | 8513.005 | 5940.675 | 5353.26 |
| 1568.34 | 1625.925 | 3866.115 | 3975.155 | 3546.665 | 2500.985 | 1819.895 | 1493.43 | 3213.315 | 2697.165 |
| 1711.92 | 2051.65 | 5201.13 | 5733.005 | 7469.38 | 5448.07 | 2736.42 | 1909.75 | 5047.76 | 4696.915 |
| 1543.78 | 1509.065 | 7446.305 | 6661.67 | 7421.185 | 5748.105 | 2737.025 | 2213.03 | 5438.9 | 3962.76 |
| Data 6:Cy5 | Data 6:Cy3 | Data 7:Cy5 | Data 7:Cy3 | Data 8:Cy5 | Data 8:Cy3 | Data 9:Cy5 | Data 9:Cy3 | Data 10:Cy5 | Data 10:Cy3 |
| 490.83 | 766.79 | 1951.36 | 643.895 | 1394.385 | 864.035 | 1860.32 | 816.285 | 1217.765 | 476.08 |
| 3460 | 7300.145 | 46388.86 | 7799.63 | 16211.28 | 4358.465 | 13368.255 | 8180.1 | 12252.13 | 3739.46 |
| 1537.37 | 2159.51 | 9414.69 | 5731.56 | 4835.385 | 6094.055 | 4225.02 | 4385.155 | 3575.365 | 2021.495 |
| 12385.45 | 17411.06 | 17518.285 | 11758.93 | 5784.325 | 7554 | 5565.72 | 5374.73 | 5345.315 | 5169.935 |
| 18253.885 | 26824.05 | 40435.225 | 31453.775 | 13877.33 | 16146.425 | 11795.21 | 15179.65 | 8878.185 | 9470.42 |
| 52524.645 | 50976.35 | 51183.015 | 50683.165 | 36053.8 | 51188.445 | 22270.15 | 34780.995 | 13823.49 | 22279.04 |
| 581.29 | 1543.525 | 2204.185 | 761.2 | 1976.22 | 1175.035 | 1687.74 | 2585.625 | 1204.06 | 1362.435 |
| 3010.385 | 4352.9 | 4801.81 | 1989.52 | 1805.71 | 1464 | 1891.12 | 1435.15 | 1409.37 | 882.935 |
| 3983.195 | 4958.98 | 15559.95 | 11564.35 | 3810.63 | 4789.925 | 4825.21 | 3636.86 | 2700.22 | 1897.505 |
| 4166.985 | 6419.74 | 33419.45 | 36355.125 | 15885.58 | 32896.075 | 3706.825 | 6563.48 | 2247.335 | 3390.52 |
| 222.005 | 776.965 | 2421.84 | 4410.705 | 663.48 | 2119.91 | 3328.07 | 3951.92 | 1543.835 | 1417.085 |
| 3116.345 | 2597.35 | 27094.29 | 30877.915 | 8723.61 | 17584.1 | 7821.305 | 17035.845 | 5474.02 | 10250.37 |
| 685.355 | 1597.73 | 2715.495 | 1662.045 | 2087.67 | 2325 | 1651.63 | 1221.115 | 1024.55 | 666.84 |
| 14752.935 | 21988.835 | 10358.065 | 7240.115 | 2350.455 | 2299.75 | 3293.905 | 4723.45 | 1805.795 | 3006.425 |
| 2240.985 | 4387.345 | 5453.16 | 2794.43 | 2864.24 | 2597.45 | 3341.435 | 2173.83 | 1380.33 | 972.235 |
| 1023.295 | 1359.145 | 9399.53 | 6736.88 | 5412.96 | 11385.9 | 1587.015 | 738.725 | 1679.755 | 510.5 |
| 236.455 | 579.36 | 4882.12 | 1323.59 | 2209.065 | 1391.25 | 1879.63 | 1143.625 | 1692.31 | 274.475 |
| 39212.65 | 37390.115 | 40267.92 | 32051.96 | 14599.87 | 20564.985 | 17626.685 | 16700.78 | 13837.68 | 14390.375 |
| 3905.7 | 6139.7 | 19787.235 | 10874.535 | 8241.8 | 9874.54 | 3343.425 | 2227.44 | 2311.23 | 1431.66 |
| 2887.705 | 4635.765 | 15557.245 | 5521.925 | 5663.665 | 4694.975 | 4979.52 | 2066.76 | 5027.855 | 1495.27 |
| 25398.965 | 25301.39 | 14403.03 | 15145.21 | 7143.91 | 13491.315 | 6062.165 | 7995.21 | 3653.275 | 6418.075 |
| 1566.04 | 2777.525 | 5436.225 | 5680.505 | 3081.48 | 4316.505 | 1218.09 | 4933.72 | 1522.455 | 3582.625 |
| 5267.015 | 5800.41 | 15693.4 | 12537.78 | 5428.745 | 6585.57 | 7443.605 | 6019.505 | 4907.915 | 3666.95 |
| 15177.39 | 26715.235 | 17784.245 | 19625.575 | 8300.605 | 13412.75 | 6428.88 | 22581.98 | 5350.38 | 10041.955 |
| 2636.665 | 2577.665 | 4554.51 | 2564.735 | 3288.46 | 3729.42 | 2423.845 | 2609.03 | 1974.66 | 1483.5 |
| 676.625 | 1254.165 | 4091.055 | 1334.12 | 1511.76 | 1036.02 | 1314.99 | 713.51 | 859.26 | 639.8 |
| 4301.04 | 6398.99 | 9628.815 | 7644.405 | 3582 | 4450.155 | 5466.15 | 10116.255 | 3936.1 | 5794.9 |
| 6133.845 | 6666.685 | 7872.565 | 5442.765 | 3045.17 | 3897.925 | 3463.87 | 1959.855 | 2709.535 | 1078.98 |
| 3704.02 | 5888.605 | 5868.805 | 2834.42 | 3394.89 | 3477.265 | 2363.56 | 3353.865 | 1664.37 | 1560.59 |
| 1217.76 | 1742.45 | 3751.995 | 6419.36 | 1880.74 | 4754.68 | 2118.59 | 4718.515 | 1753.895 | 4577.605 |
| 17094.645 | 27348.455 | 37616.815 | 12642.75 | 18200.645 | 11711.465 | 16167.745 | 7767.91 | 17505.905 | 3831.62 |
| 21681.39 | 36694.03 | 57587.925 | 25379.92 | 29360.71 | 15560.485 | 29497.695 | 16880.085 | 33297.515 | 14914.545 |
| 14243.11 | 24710.615 | 54796.205 | 32148.775 | 26117.195 | 17387.195 | 19700.74 | 12021.8 | 24995.14 | 11529.875 |
| 2972.57 | 4515.085 | 9823.34 | 3220.41 | 2397.475 | 1220.945 | 4277.435 | 1994.645 | 3237.575 | 1296.365 |
| 999.065 | 1506.255 | 39641.32 | 8411.865 | 12902.83 | 5473.14 | 9567.745 | 4656.385 | 10999.42 | 3773.9 |
| 176.81 | 648.645 | 4524.205 | 1723.715 | 2054.315 | 1449.315 | 1625.96 | 1139.205 | 1309.245 | 571.375 |
| 1609.74 | 2621.535 | 1692.975 | 753.57 | 1003.63 | 619.07 | 1414.215 | 668.63 | 701.4 | 330.045 |
| 45274.735 | 49554.975 | 3466.855 | 3223.005 | 1579.875 | 2492.08 | 2291.115 | 3624.155 | 1100.715 | 1603.765 |
| 1004.91 | 1500.36 | 7734.875 | 5699.545 | 4127.24 | 5070.885 | 1753.57 | 3340.55 | 2216.04 | 3545.115 |
| 1886.37 | 3025.905 | 8686.005 | 7325.39 | 3413.355 | 4406.605 | 2474.97 | 5040.385 | 2423.84 | 3182.765 |
| 7796.15 | 15694.73 | 14418.235 | 8242.165 | 6818.53 | 7568.475 | 4836.11 | 9512.495 | 6990.595 | 8861.385 |
| 4801.24 | 4682.365 | 10851.725 | 6889.435 | 4968.735 | 6854.645 | 4677.845 | 5241.825 | 5585.835 | 5765.025 |
| 2139.115 | 2379.74 | 8495.44 | 7646.065 | 4411.11 | 8359.45 | 3091.66 | 4865.665 | 2687.7 | 2474.36 |
| 882.24 | 2086.885 | 4398.01 | 1844.18 | 2524.385 | 1852.165 | 2165.565 | 3439.73 | 1118.23 | 553.495 |
| 5708.125 | 3529.485 | 9439.18 | 10084.31 | 7128.185 | 13697.73 | 3679.57 | 7066.325 | 2481.99 | 3123.86 |
| 8801.56 | 17554.985 | 1729.335 | 423.87 | 1587.935 | 967.23 | 961.925 | 1248.185 | 731.425 | 778.28 |
| 5110.4 | 7611.65 | 8393.74 | 4708.575 | 2406.325 | 2588.96 | 4350.775 | 4276.33 | 3327.355 | 2162.75 |
| 7847.255 | 8888.265 | 17383.265 | 20911.92 | 7216.61 | 17328.67 | 4234.57 | 6288.02 | 5385.085 | 6896.76 |
| 585.12 | 1774.145 | 3882.535 | 1951.86 | 1895.69 | 1510.915 | 3773.775 | 2621.445 | 1035.535 | 905.94 |
| 219.92 | 553.475 | 4808.965 | 3143.36 | 2422.865 | 3169.68 | 2013.68 | 4571.175 | 1644.22 | 1369.605 |
| 10153.11 | 15586.72 | 34789.06 | 55305.02 | 14033.165 | 48324.215 | 6914.33 | 23830.63 | 9436.82 | 23915.065 |
| 11087.47 | 18804.735 | 19989.775 | 10629.475 | 11187.675 | 12389.42 | 6109.325 | 15515.995 | 10255.15 | 12376.725 |
| 57259.575 | 53927.98 | 41273.99 | 30591.625 | 30829.225 | 35493.83 | 23233.78 | 31404.32 | 16555.325 | 20031.21 |
| 5472.99 | 7318.565 | 3702.69 | 1674.26 | 1867.605 | 1549.945 | 2763.355 | 2262.04 | 1334.325 | 870.54 |
| 14126.915 | 18956.33 | 14481.145 | 9666.55 | 5195.93 | 4363.365 | 3642.24 | 3970.47 | 2182.22 | 1842.685 |
| 1173.75 | 1198.54 | 29185.315 | 12270.96 | 10873.115 | 9601.52 | 10454.61 | 4177.645 | 13209.78 | 3661.72 |
| 1183.625 | 2293.15 | 10541.325 | 2843.91 | 4299.545 | 2465.14 | 3775.975 | 3694.23 | 4664.73 | 1908.145 |
| 23201.285 | 41703.28 | 36517.58 | 12469.54 | 13198.035 | 13885.445 | 15228.815 | 11196.48 | 18303.7 | 7777.455 |
| 10972.48 | 19055.66 | 25831.94 | 9563.655 | 16135.265 | 10472.745 | 14217.57 | 16206.985 | 15812.455 | 9756.26 |
| 57206.74 | 55111.035 | 39820.885 | 18077.905 | 24471.58 | 20848.495 | 24828.55 | 28335.015 | 31714.235 | 18241.06 |
| 2400.185 | 4416.35 | 6723.75 | 6077.845 | 1961 | 3258.22 | 2821.135 | 5423.67 | 1257.145 | 2333.415 |
| 3058.51 | 5576.4 | 5512.215 | 3094.825 | 2867 | 3457.41 | 2802.8 | 5839.54 | 3542.115 | 4617.315 |
| 15490.105 | 22493.22 | 12025.01 | 10398.855 | 3873.27 | 6592.605 | 3128.71 | 8744.605 | 1869.12 | 4568.575 |
| 52373.045 | 54345.455 | 40405.355 | 41302.755 | 21033.375 | 33339.935 | 17145.74 | 47032.135 | 16178.45 | 26718.395 |
| 570.99 | 1377.87 | 2722.785 | 1339.535 | 1911.755 | 1538.14 | 1577.475 | 923.08 | 975.085 | 382.945 |
| 15376.14 | 21873.435 | 40514.52 | 40685.97 | 19639.89 | 36988.48 | 10588.49 | 32361.905 | 23103.355 | 37476.63 |
| 13863.355 | 19114.31 | 41433.38 | 43732.875 | 19134.245 | 30446.66 | 15721.23 | 33549.605 | 19987.22 | 29885.715 |
| 38846.125 | 46892.16 | 39316.295 | 41221.505 | 18861.62 | 36691.2 | 11596.035 | 37909.85 | 13818.325 | 45696.66 |
| 4178.48 | 3981.975 | 5750.84 | 2992.265 | 2145.19 | 2035.77 | 2486.01 | 2552.67 | 1284.95 | 1163.315 |
| 13538.605 | 16767.275 | 17574.965 | 7506.445 | 5863.755 | 4731.425 | 8230.225 | 5061.59 | 6478.955 | 3373.34 |
| 5828.355 | 9870.81 | 3291.465 | 1797.785 | 1949.575 | 1831.45 | 2601.89 | 3614.15 | 1753.335 | 1692.99 |
| 456.105 | 1733.155 | 5357.45 | 1543.015 | 2032.735 | 1355.245 | 2698.455 | 2213.625 | 1403.16 | 538.94 |
| 940.64 | 1493.34 | 2366.28 | 799.335 | 1571.535 | 1142.225 | 1935.995 | 1105.27 | 599.12 | 304.01 |
| 1097.785 | 2222.535 | 1874.47 | 584.205 | 1077.875 | 662.895 | 1718.945 | 941.695 | 662.885 | 282.12 |
| 456.62 | 715.795 | 3451.505 | 2718.995 | 2750.25 | 3787.56 | 1282.485 | 2050.28 | 817.745 | 844.86 |
| 977.86 | 1791.705 | 3867.22 | 2945.025 | 2214.565 | 2685.685 | 1751.57 | 4367.39 | 1402.11 | 1544.235 |
| 1218.48 | 1365.385 | 5051.04 | 3864.22 | 3413.16 | 5530.515 | 1214.3 | 2236.64 | 1199.215 | 1085.945 |
| 5773.89 | 6862.155 | 16770.29 | 22426.835 | 6817.98 | 16606.745 | 5852.94 | 12912.6 | 5521.48 | 10937.845 |
| 1948.105 | 2727.535 | 23655.825 | 7923.23 | 6549.05 | 4497.6 | 1974.65 | 690.705 | 2456.585 | 1200.12 |
| 3390.48 | 5928.72 | 2870.815 | 2378.105 | 1938.21 | 2210.9 | 1073.24 | 444.63 | 854.54 | 264.46 |
| 5405.515 | 7418.14 | 13645.82 | 8229.665 | 4599.75 | 5598.99 | 4743.265 | 5192.145 | 2283.43 | 2661.875 |
| 7348.435 | 12151.03 | 20912.315 | 31162.68 | 8106.285 | 14992.545 | 7054.285 | 20548.805 | 4820.61 | 8415.435 |
| 4165.66 | 8716.455 | 2690.525 | 1388.185 | 2107.06 | 1303.265 | 2190.175 | 1544.85 | 1044.55 | 371.36 |
| 16156.18 | 22370.56 | 6252.49 | 6459.315 | 3234.855 | 5604.955 | 2477.415 | 4777.27 | 1961.4 | 6013.015 |
| 1430.945 | 2487.925 | 4354.6 | 1940.515 | 2661.85 | 2303.455 | 2907.38 | 2341.515 | 1408.935 | 777.44 |
| 1591.385 | 3736.995 | 6789.925 | 2914.61 | 3773.25 | 3249.235 | 4272.815 | 6478.35 | 5908.395 | 3035.945 |
| 5045.695 | 6177.005 | 12208.26 | 8924.4 | 4707.595 | 5874.915 | 3627.36 | 3964.065 | 4366.55 | 4693.01 |
| 4056.605 | 7171.755 | 16152.41 | 8905.62 | 5843.585 | 6258.535 | 5717.8 | 5375.49 | 4031.14 | 2779.74 |
| 24250.505 | 28285.755 | 14261.2 | 32485.125 | 5866.705 | 22541.755 | 5331.195 | 18221.27 | 4299.655 | 14083.2 |
| 4327.405 | 5450.99 | 18696.595 | 15947.315 | 5173.385 | 8451.565 | 4505.35 | 2977.235 | 2881.665 | 2272.62 |
| 4729.045 | 5504.19 | 12906.02 | 6830.56 | 7871.94 | 8662.49 | 6265.985 | 5178.15 | 4810.56 | 3132.165 |
| 905.94 | 981.675 | 13022.8 | 7130.89 | 4973.755 | 4780.365 | 5511.99 | 9730.115 | 4975.115 | 6946.38 |
| 1004.62 | 1863.73 | 6482.455 | 6418.99 | 3056.3 | 5040.67 | 2135.685 | 3181.395 | 1577.525 | 1010.685 |
| 45826.79 | 47148 | 49749.105 | 24867.305 | 40568.49 | 33988.11 | 29254.27 | 25953.61 | 31262.91 | 15090.32 |
| 651.75 | 1410.33 | 5235.055 | 5210.415 | 2433.405 | 5775.675 | 2778.605 | 2398.87 | 1587.07 | 877.365 |
| 2588.025 | 3440.28 | 9410.255 | 5763.375 | 3486.905 | 3993.255 | 3395.005 | 7135.535 | 1913.45 | 2260.615 |
| 1123.795 | 1899.195 | 3358.56 | 1290.445 | 1529.43 | 710.75 | 2184.39 | 865.9 | 829.8 | 301.24 |
| 611.23 | 1953.29 | 4697.205 | 1890.77 | 2502.625 | 1774.725 | 2620.015 | 3320.325 | 2556.095 | 1576.97 |
| 4402.9 | 8359.125 | 15804.32 | 7672.45 | 6536.015 | 6272.15 | 4048.79 | 5381.79 | 5664.7 | 5025.295 |
| 1394.33 | 2134.12 | 4355.13 | 2110.06 | 2394.145 | 2094.89 | 1566.2 | 2312.69 | 2135.27 | 1432.895 |
| 10349.46 | 10116.865 | 11073.405 | 5061.67 | 5817.135 | 5637.23 | 4244.44 | 4367.675 | 7678.235 | 3238.16 |
| 400.915 | 787.02 | 2804.455 | 1366.185 | 1828.555 | 1489.68 | 1300.51 | 4030.495 | 713.475 | 561.855 |
| 721.44 | 1615.35 | 2052.815 | 757.35 | 1222.23 | 876.89 | 1272.365 | 2120.32 | 806.545 | 438.575 |
| 15135.18 | 26845.825 | 23471.435 | 9339.39 | 15767.545 | 8774.225 | 14878.5 | 14713.015 | 12017.76 | 6861.14 |
| 1103.725 | 2814.145 | 9378.765 | 1645.605 | 3141.74 | 1280.88 | 2415.22 | 660.515 | 2229.53 | 267.12 |
| 10896.42 | 11813.12 | 14944.065 | 14130.675 | 4653.18 | 7389.86 | 6832.865 | 8168.38 | 4017.235 | 4562.085 |
| 3542.315 | 6487.825 | 11124.88 | 8030.29 | 2645.42 | 3685.19 | 3411.635 | 7243.615 | 2223.65 | 4812.985 |
| 1331.345 | 1778.975 | 4403.715 | 4767.185 | 3124.245 | 6493.86 | 960.64 | 1832.76 | 692.88 | 830.385 |
| 456.205 | 1335.765 | 7711.64 | 9818.855 | 2475.795 | 3723.49 | 2351.205 | 4814.99 | 1884.785 | 2827.365 |
| 756.47 | 1844.04 | 2987.295 | 673.085 | 2227.4 | 1030.595 | 1322.57 | 1091.375 | 898.845 | 486.54 |
| 5317.26 | 6649.92 | 14298.06 | 9022.03 | 7070.985 | 11654.885 | 7083.595 | 6191.1 | 4820.755 | 4827.12 |
| 869.705 | 1938.185 | 3602.665 | 2769.62 | 1407.165 | 1875.055 | 2356.995 | 3699.405 | 1086.77 | 603.005 |
| 1248.655 | 2437.74 | 2982.61 | 1147.34 | 1859.75 | 1060.365 | 2014.635 | 1015.08 | 1200.815 | 402.73 |
| 2487.46 | 2336.03 | 9650.51 | 3777.305 | 3855.745 | 3626.71 | 1582.435 | 1263.935 | 961.615 | 647.13 |
| 559.975 | 910.205 | 9156.43 | 8306.395 | 3734.105 | 6523.065 | 5005.21 | 11049.98 | 4092.25 | 7077.02 |
| 4035.615 | 4933.69 | 24597.105 | 6631.2 | 7825.11 | 6055.235 | 7085.52 | 8213.175 | 8814.195 | 5061.855 |
| 2223.32 | 3198.205 | 5176.075 | 3460.23 | 3015.295 | 3385.685 | 3800.58 | 3235.3 | 2727.085 | 2353.655 |
| 2526.85 | 3293.21 | 6317.97 | 6861.485 | 3532.695 | 5777.475 | 2004.715 | 2936.16 | 2555.71 | 3675.855 |
| 5041.09 | 8800.92 | 6948.965 | 2309.705 | 2997.05 | 1628.575 | 2524.24 | 3582.085 | 2350.62 | 2289.86 |
| 690.5 | 1479.62 | 3541.75 | 1970.285 | 1987.89 | 1462.3 | 1615.895 | 2056.435 | 1283.315 | 756.085 |
| 5404.96 | 6323.625 | 25667.435 | 36810.57 | 13988.98 | 33837.68 | 7941.255 | 22191.075 | 10970.71 | 26224.725 |
| 4501.465 | 10193.035 | 6084.845 | 5365.645 | 2384.785 | 3471.525 | 1491.265 | 3555.51 | 1189.57 | 1217.43 |
| 2931.16 | 2326.85 | 8689.535 | 38903.43 | 6149.18 | 45501.345 | 5483.995 | 52557.42 | 6263.15 | 41871.66 |
| 8324.83 | 13233.575 | 3868.475 | 2279.925 | 1884.935 | 2003.205 | 2066.875 | 3823.235 | 1525.295 | 2412.415 |
| 7692.46 | 11965.72 | 10178.97 | 11611.865 | 5256.18 | 10447.05 | 4134.735 | 15713.215 | 3520.52 | 4817.09 |
| 716.925 | 1674.685 | 3877.515 | 1528.22 | 2041.105 | 1226.015 | 1715.215 | 1333.16 | 1899.205 | 821.38 |
| 2156.025 | 4095.28 | 12032.41 | 883.63 | 4663.25 | 894.01 | 3460.525 | 2095.72 | 3371.53 | 1116.62 |
| 415.035 | 683.065 | 2294.555 | 954.715 | 1876.015 | 1442.275 | 1474.055 | 1554.14 | 912.585 | 669.235 |
| 5553.555 | 9639.925 | 15471.06 | 8108.165 | 4820.87 | 5019.625 | 3329.01 | 6600.22 | 4559.93 | 4508.005 |
| 5576.075 | 7910.535 | 11307.7 | 13731.88 | 6532.47 | 14577.305 | 3405.505 | 9416.07 | 4168.12 | 9519.355 |
| 2120.275 | 3680.6 | 9032.805 | 4620.725 | 2562.79 | 2369.675 | 1646.06 | 3770.59 | 1626.785 | 1460.365 |
| 882.07 | 1764.62 | 3403.48 | 1086.805 | 2375.345 | 1307.86 | 1938.08 | 1224.01 | 1521.54 | 754.87 |
| 3303.135 | 3104.925 | 5209.435 | 6229.355 | 3648.425 | 7319.395 | 2125.865 | 2416.395 | 1092.625 | 1058.565 |
| 1176.615 | 1798.82 | 8114.445 | 1904.565 | 3310.21 | 1528.485 | 4206.16 | 1178.905 | 3960.055 | 788.47 |
| 625.165 | 1145.265 | 13159.005 | 29497.43 | 6760.38 | 28872.12 | 4421.83 | 24787.39 | 4059.18 | 23282.07 |
| 921.97 | 1344.33 | 4202.95 | 3292.08 | 1674.93 | 1787.305 | 1827.76 | 3239.375 | 735.32 | 818.51 |
| 20285.46 | 34121.19 | 26802.645 | 10811.235 | 22054.295 | 13204.305 | 25338.29 | 22598.36 | 25082.58 | 13486.525 |
| 16041.535 | 20115.34 | 50418.665 | 44608.795 | 31741.755 | 43566.32 | 20667.43 | 24118.49 | 22071.36 | 24141.935 |
| 50356.285 | 48572.21 | 39936.455 | 30143.775 | 24279.585 | 30148.16 | 40994.6 | 38709.56 | 24641.355 | 24359.245 |
| 10199.47 | 12049.76 | 27785.3 | 15697.505 | 13561.415 | 13974.92 | 14028.15 | 13474.125 | 14555.715 | 10423.205 |
| 3949.97 | 5581.755 | 11658.115 | 11262.325 | 5148.02 | 10811.755 | 6291.825 | 7554.97 | 6731.08 | 7472.025 |
| 5354.165 | 9239.495 | 3811.89 | 3206.93 | 2495.78 | 3949.05 | 2293.29 | 1749.05 | 1662.88 | 1080.095 |
| 7990.74 | 13992.445 | 14727.02 | 4353.58 | 7082.775 | 3656.31 | 7370.05 | 5931.11 | 6617.55 | 2592.995 |
| 530.18 | 1323.205 | 10389.04 | 2667.425 | 3747.71 | 2162.8 | 3274.935 | 1897.1 | 3380.05 | 1429.05 |
| 6883.905 | 10075.365 | 9604.415 | 7188.205 | 5075.035 | 8340.535 | 2291.685 | 5065.25 | 2196.72 | 2459.25 |
| 19231.875 | 33986.06 | 17058.925 | 6474.565 | 9830.795 | 8467.145 | 5533.865 | 10343.34 | 7475.665 | 5472.76 |
| 726.725 | 2078.92 | 4174.205 | 18892.105 | 1537.4 | 6916.91 | 9552.005 | 40341.185 | 1628.025 | 11263.6 |
| 253.555 | 551.88 | 2124.12 | 702.335 | 1574.685 | 818.26 | 1705.31 | 2395.99 | 1334.285 | 669.46 |
| 475.425 | 689.935 | 2603.385 | 823.64 | 1805.555 | 995.05 | 1256.81 | 1208.115 | 724.6 | 306.595 |
| 893.615 | 1868.935 | 2724.655 | 2418.785 | 1632.56 | 2273.325 | 2840.8 | 3512.37 | 1658.025 | 1460.3 |
| 187.125 | 513.015 | 3246.805 | 3549.03 | 1700.4 | 3499.445 | 1700.445 | 3581.19 | 1117.115 | 1093.915 |
| 41612.93 | 42579.55 | 51971.8 | 17730.72 | 19359.17 | 11941.855 | 19812.765 | 6029.255 | 23441.94 | 6267.455 |
| 25951.955 | 37975.885 | 23722.99 | 52777.265 | 14697.585 | 58022.505 | 9924.475 | 41496.78 | 8490.91 | 32274.575 |
| 244.065 | 535.445 | 4830.93 | 520.48 | 2769.315 | 788.41 | 1887.78 | 638.815 | 1601.17 | 233.455 |
| 380.24 | 116.265 | 2783.13 | 893.535 | 1558.77 | 895.415 | 955.52 | 856.98 | 842.685 | 373.58 |
| 37497.005 | 46398.59 | 58079.1 | 48891.39 | 57616.6 | 41582.215 | 48314.49 | 23652.32 | 52484.18 | 19664.225 |
| 534.605 | 1166.465 | 7595.61 | 6499.055 | 5603.285 | 7581.885 | 1522.615 | 6043.105 | 1947.645 | 2358.91 |
| 830.74 | 1972.645 | 3245.72 | 1179.025 | 1397.545 | 995.515 | 2413.39 | 2029.905 | 1286.67 | 552.51 |
| 28423.045 | 47297.215 | 60439.165 | 56888.075 | 55673.08 | 59696.715 | 21080.165 | 50360.36 | 41631.27 | 40938.525 |
| 3873.55 | 6361.73 | 7883.115 | 1951.085 | 3333.42 | 1729.7 | 4874.25 | 3623.175 | 3886.99 | 1895.6 |
| 4033.95 | 5296.985 | 7298.86 | 2182.24 | 3058.965 | 1890.6 | 3190.505 | 1773.18 | 1939.985 | 862.95 |
| 10416.295 | 11845.175 | 9017.62 | 6242.815 | 2872.38 | 3052.78 | 3008.75 | 2989.945 | 2521.67 | 3820.835 |
| 348.05 | 1671.99 | 5203.775 | 1586.68 | 2455.55 | 1611.26 | 1771.815 | 2916.315 | 1529.545 | 669.415 |
| 3690.3 | 5070.46 | 7293.405 | 5256.835 | 3855.3 | 4490.5 | 1915.75 | 2738.405 | 1810.595 | 2649.695 |
| 10290.295 | 21709.47 | 39936.31 | 29342.745 | 23161.265 | 29131.645 | 8532.685 | 17968.935 | 8971.77 | 8230.53 |
| 6988.845 | 4851.61 | 4060.615 | 871.85 | 2058.27 | 946.51 | 2922.85 | 1162.73 | 1497.63 | 1080.075 |
| 2769.195 | 3547.785 | 12241.68 | 13170.71 | 4934.81 | 8097.45 | 3222.4 | 5079.46 | 4312.125 | 7066.24 |
| 17647.685 | 27284.985 | 37419.895 | 26100.195 | 15547.84 | 21924.395 | 12400.255 | 14785.95 | 13603.425 | 14863.275 |
| 4734.07 | 4525.365 | 18393.99 | 43451 | 9847.725 | 47430.305 | 7328.695 | 36308.84 | 7296.04 | 29931.26 |
| 2898.47 | 4684.23 | 11072.52 | 6555.995 | 5832.475 | 6426.81 | 7050.785 | 6691.53 | 5830.54 | 3951.71 |
| 8348.15 | 9727.66 | 37125.765 | 17677.015 | 13153.745 | 13380.9 | 15664.735 | 9281.895 | 15733.795 | 8749.925 |
| 14470.115 | 14914.695 | 54191.64 | 41125.605 | 33040.72 | 37325.92 | 31421.48 | 28878.825 | 37732.335 | 26837.97 |
| 23231.31 | 35681.05 | 16794.405 | 11885.445 | 5925.09 | 7437.635 | 3450.09 | 5889.455 | 3968.225 | 4989.705 |
| 3087.27 | 4386.54 | 4620.405 | 3076.275 | 3501.175 | 4802.09 | 1826.56 | 4810.27 | 1867.38 | 1555.47 |
| 10069.06 | 10535.735 | 23462.435 | 22466.065 | 8208.165 | 14580.355 | 9735.32 | 9449.36 | 7199.965 | 8216.115 |
| 32873.415 | 38736.76 | 58143.315 | 54638.725 | 39427.515 | 40823.055 | 41500.485 | 41334.305 | 36588.94 | 28871.175 |
| 2192.255 | 5027.725 | 2256.535 | 1110.91 | 1198.97 | 1207.69 | 2317.02 | 4627.94 | 940.08 | 1393.03 |
| 398.64 | 1005.09 | 2823.2 | 924.655 | 1720.185 | 1065.825 | 1720.215 | 1529.63 | 1433.53 | 953.935 |
| 522.355 | 1501.105 | 21758.99 | 4061.135 | 6132.21 | 2561.425 | 6259.21 | 1678.16 | 7253.005 | 1285.67 |
| 17282.6 | 18107.895 | 20995.415 | 21003.79 | 7934.595 | 13708.895 | 5912.69 | 7308.92 | 3218.01 | 5682.86 |
| 2191.195 | 3795.435 | 7120.955 | 3784.815 | 3569.685 | 3177.58 | 3512.28 | 5862.76 | 2968.755 | 3542.035 |
| 3307.035 | 5182.785 | 15818.125 | 15184.585 | 6983.47 | 12351.065 | 1201.255 | 636.725 | 846.795 | 367.34 |
| 4223.685 | 7168.66 | 33010.45 | 16878.82 | 12811.23 | 13283 | 8427.845 | 5071.005 | 10496.255 | 3292.925 |
| 2827 | 2299.2 | 9551.64 | 10883.97 | 5279.75 | 13573.99 | 2333.81 | 7237.195 | 2906.625 | 4120.29 |
| 13745.795 | 17046.4 | 14976.1 | 40925.905 | 6665.44 | 30335.79 | 4309.325 | 24689.37 | 4729.02 | 25712.315 |
| 9032.525 | 11585.14 | 16398.06 | 18859.385 | 8403.765 | 21082.16 | 4204.325 | 6072.94 | 5062.775 | 7018.76 |
| 1051.005 | 2546.18 | 3875.96 | 974.63 | 1611.65 | 764.78 | 1911.765 | 771.35 | 1285.545 | 423.65 |
| 3353.355 | 6059.08 | 7744.09 | 3007.435 | 4375.755 | 3169.32 | 1133.625 | 527.24 | 884.015 | 306.51 |
| 33083.335 | 44337.36 | 42835.495 | 34951.485 | 15317.605 | 17069.52 | 15633.83 | 17550.135 | 12233.085 | 7628.94 |
| 23037.415 | 21084.82 | 31016.365 | 26780.455 | 8053.2 | 11682.58 | 11252.965 | 8271.54 | 7837.12 | 7251.575 |
| 5752.255 | 8729.84 | 17844.845 | 8583.835 | 7767.095 | 7531.65 | 5649.005 | 10926.08 | 6847.26 | 4146.48 |
| 22697.95 | 33799.905 | 41318.42 | 40199.045 | 16376.295 | 30823.685 | 10408.805 | 26926.11 | 11425.375 | 19456.55 |
| 785.29 | 1405.495 | 1789.66 | 1233.44 | 1181.735 | 1333.725 | 1387.17 | 393.215 | 664.085 | 210.75 |
| 2601.25 | 2149.875 | 12994 | 56008.795 | 6656.59 | 57476.58 | 5805.035 | 39211.955 | 3183.795 | 24359.675 |
| 19438.805 | 29929.495 | 13865.14 | 6973.805 | 6811.245 | 9377.975 | 11912.935 | 10168.22 | 14563.835 | 6027.185 |
| 5848.5 | 7382.825 | 4525.065 | 7616.62 | 3111.88 | 8942.825 | 3066.915 | 13342.775 | 4197.04 | 13698.445 |
| 20635.055 | 44604.815 | 32532.84 | 15257.695 | 7815.025 | 7181.5 | 7196.995 | 31764.655 | 6155.195 | 7566.295 |
| 5469.855 | 9218.48 | 13335.91 | 15690.3 | 7921.395 | 16811.605 | 3518.435 | 9990.385 | 4840.025 | 12319.195 |
| 1354.495 | 2193.53 | 4207.85 | 2864.225 | 2644.01 | 2689.295 | 2380.47 | 3654.69 | 1016.17 | 944.795 |
| 8875.455 | 11937.655 | 6690.42 | 4552.985 | 3493.76 | 3961.07 | 3298.205 | 6440.235 | 4200.315 | 6701.595 |
| 932.91 | 1873.47 | 3501.825 | 1501.25 | 2292.95 | 1644.275 | 3173.9 | 2593.835 | 1820.59 | 1628.62 |
| 2119.275 | 3086.52 | 8536.63 | 2961.815 | 2997.87 | 1962.345 | 3842.385 | 3808.6 | 1994.88 | 1516.405 |
| 4489.94 | 5594.805 | 9897.99 | 5427.69 | 3979.74 | 4059.63 | 4233.355 | 2972.365 | 2488.57 | 1893.405 |

| Data 11:Cy5 | Data 11:Cy3 | Data 12:Cy5 | Data 12:Cy3 | Data 13:Cy5 | Data 13:Cy3 |
|---|---|---|---|---|---|
| 2499.845 | 983.21 | 1493.985 | 457.925 | 615.38 | 965.92 |
| 15077.045 | 6648.705 | 25176.09 | 4754.04 | 6018.96 | 8219.44 |
| 4328.785 | 3404.88 | 6839.745 | 3728.49 | 1778.335 | 1678.545 |
| 6540.19 | 6098.725 | 7679.17 | 4501.245 | 3076.215 | 3875.69 |
| 12036.26 | 15609.55 | 14924.075 | 10562.375 | 8011.55 | 11757.46 |
| 25254.085 | 42516.34 | 31446.16 | 31498.39 | 38014.2 | 39695.93 |
| 1995.165 | 1595.445 | 1825.58 | 1297.01 | 641.985 | 3043.28 |
| 2747.855 | 1985.13 | 1974.59 | 942.04 | 909.5 | 1888.24 |
| 5218.25 | 3828.105 | 4012 | 2164 | 2165.215 | 2456.46 |
| 4225.95 | 7147.025 | 13148.105 | 12881.275 | 11875.455 | 11465.525 |
| 2875.1 | 3772.875 | 2937.02 | 4171.87 | 617.77 | 1847.155 |
| 7766.985 | 15900.23 | 10830.785 | 15804.705 | 5503.945 | 19154.92 |
| 1994.185 | 1286.52 | 1630.845 | 777.85 | 1383.255 | 2216.63 |
| 3486.18 | 3913.53 | 3587.825 | 2758.925 | 1340.18 | 10267.22 |
| 2732.67 | 2204.505 | 2216.07 | 1353.03 | 1235.555 | 3287.3 |
| 2252.88 | 863.26 | 1837.425 | 528.935 | 3443.415 | 10310.73 |
| 2246.725 | 642.345 | 3335.575 | 538.57 | 1242.23 | 1283.64 |
| 14889.195 | 16772.405 | 25802.115 | 19250.655 | 7674.795 | 4894.29 |
| 3158.76 | 1966.515 | 4119.75 | 1860.27 | 5442.06 | 7761.1 |
| 6058.315 | 2224.655 | 9759.575 | 1888.91 | 3426.925 | 2499.44 |
| 4986.695 | 8611 | 7182.445 | 7299.87 | 3818.705 | 8022.905 |
| 2711.99 | 5075.45 | 2658.515 | 3983.61 | 1338.13 | 1736.935 |
| 5854.295 | 4822.55 | 9037.795 | 5302.7 | 2318.11 | 2024.44 |
| 5803.89 | 16390.535 | 10814.585 | 20855.81 | 4607.695 | 4280.86 |
| 3776.425 | 3158.06 | 3035.475 | 2177.035 | 1569.125 | 1574.115 |
| 2032.975 | 849.91 | 1168.98 | 500.795 | 965.445 | 2645.155 |
| 5179.63 | 7863.065 | 7975.52 | 8446.025 | 1876.15 | 3279.3 |
| 4481.77 | 2292.96 | 4384.695 | 1325.525 | 1248.39 | 2145.735 |
| 2624.82 | 2574.895 | 3559.625 | 3106.595 | 1432.595 | 4102.175 |
| 3042.33 | 5300.82 | 2301.045 | 4025.835 | 598.63 | 5553.51 |
| 17453.775 | 6589.135 | 30926.04 | 7922.09 | 12323.795 | 9969.495 |
| 37847.9 | 20526.305 | 53409.78 | 15830.025 | 23142.29 | 7292.025 |
| 32837.545 | 17321.605 | 37962.62 | 10898.915 | 23064.405 | 9864.585 |
| 4614.68 | 1995.66 | 5719.195 | 1405.065 | 1668.795 | 1625.895 |
| 12129.87 | 6026.305 | 15599.94 | 4292.655 | 5966.06 | 4418.95 |
| 2242.49 | 1185.535 | 1998.895 | 739.005 | 1272.415 | 1556.635 |
| 1188.175 | 638.67 | 1317.715 | 509.45 | 601.35 | 862.42 |
| 2122.78 | 2419.55 | 2072.79 | 2543 | 811.21 | 7351.78 |
| 3044.65 | 5574.215 | 3194.03 | 3517.65 | 2126.635 | 3977.705 |
| 3011.215 | 4539.05 | 5321.44 | 4525.265 | 1566.82 | 2724.62 |
| 9047.335 | 14572.71 | 8823.13 | 9360.315 | 3614.295 | 12520.425 |
| 7849.79 | 6933.22 | 7408.905 | 4608.89 | 3708.52 | 6309.09 |
| 3902.385 | 4632.36 | 5243.14 | 4435.93 | 3363.81 | 2878.01 |
| 3015.03 | 1918.97 | 3817.44 | 1405.53 | 942.815 | 1152.125 |
| 4855.61 | 7890.42 | 5541.3 | 5767.915 | 17488.205 | 10309.025 |
| 1385.49 | 1133.835 | 1281.785 | 723.11 | 414.895 | 2208.11 |
| 4658.13 | 4149.665 | 5114.255 | 3398.615 | 1434.615 | 3772.47 |
| 6883.82 | 10960.25 | 6307.91 | 6139.535 | 5614.675 | 10318.89 |
| 2537.105 | 2117.895 | 2094.98 | 1613.84 | 851.305 | 1375.155 |
| 2026.63 | 2849.47 | 3512.39 | 6750.96 | 1239.605 | 2636.865 |
| 11328.345 | 36147.425 | 11845.395 | 23694.08 | 11949.615 | 24274.375 |
| 7887.205 | 12875.9 | 14023.755 | 15014.28 | 7038.14 | 28190.705 |
| 35269.9 | 41209.62 | 36419.37 | 31353.83 | 11739.085 | 35308.635 |
| 2316.78 | 1657.51 | 2423.81 | 1081.735 | 780.11 | 1638.105 |
| 3321.57 | 3816.785 | 4112.055 | 2411.075 | 3859.635 | 6438.79 |
| 12915.51 | 4660.34 | 20553.235 | 4641.63 | 9342.205 | 5016.755 |
| 4385.47 | 2668.58 | 7761.25 | 2731.86 | 2871.02 | 2898.365 |
| 17026.89 | 10318.37 | 31336.525 | 9840.075 | 10145.985 | 9767.725 |
| 21216.905 | 19256.025 | 20388.365 | 14331.52 | 9859.08 | 11412.395 |
| 37262.06 | 33024.89 | 32188.065 | 18166.54 | 20745.57 | 19307.37 |
| 2338.13 | 3119.21 | 3277.46 | 3876.76 | 1317.18 | 3501.115 |
| 3425.32 | 4184.365 | 5927.345 | 5233.155 | 2316.075 | 12202.055 |
| 3078.16 | 6951.83 | 4314.975 | 6961.86 | 3537.655 | 9130.535 |
| 16452.27 | 36748.55 | 29857.755 | 42359.12 | 10074.54 | 17481.71 |
| 1778.58 | 736.37 | 1664.405 | 492.05 | 840.28 | 2317.025 |
| 23966.075 | 49540.255 | 22937.72 | 41576.815 | 11144.035 | 27987.36 |
| 18341.18 | 34044.835 | 33751.875 | 36457.68 | 10418.61 | 7869.745 |
| 16275.505 | 40722.7 | 22297.58 | 31342.54 | 8127.535 | 47318.075 |
| 2889.09 | 2757.13 | 2513.33 | 1840.55 | 1918.05 | 1861.9 |
| 6036.225 | 3200.985 | 14056.655 | 6200.235 | 3745.305 | 1941.025 |
| 2753.125 | 3150.475 | 3332.31 | 3586.615 | 1091.38 | 3948.355 |
| 2223.645 | 1382.68 | 2027.97 | 866.93 | 784.735 | 2247.79 |
| 1475.03 | 711.335 | 1355.2 | 433.92 | 837.51 | 1123.645 |
| 1702.275 | 796.805 | 1304.275 | 524.335 | 779.205 | 1065.995 |
| 1747.28 | 1387.75 | 1977.355 | 1219.845 | 902.45 | 1453.34 |
| 2941.535 | 3512.995 | 2894.98 | 2249.835 | 923.015 | 2300.825 |
| 2283.29 | 1943.325 | 2292.535 | 1574.92 | 2635.52 | 2518.69 |
| 6685.37 | 12461.025 | 10996.95 | 17595.25 | 3003.8 | 2996.945 |
| 3076.395 | 1526.675 | 2998.295 | 887.025 | 3444.015 | 2140.37 |
| 1996.89 | 677.585 | 1229.66 | 326.58 | 854.18 | 3206.21 |
| 3492.245 | 3824.265 | 5251.895 | 4059.535 | 2273.81 | 3423.525 |
| 8551.795 | 18639.975 | 7798.06 | 18108.645 | 5434.9 | 6491.49 |
| 1423.735 | 1082.265 | 2336.65 | 1301.955 | 1230.61 | 2687.33 |
| 2454.285 | 4607.85 | 3417.44 | 4512.955 | 2006.62 | 16070.425 |
| 2992.11 | 1699.52 | 4015.54 | 1567.075 | 1079.425 | 1315.04 |
| 6390.69 | 5235.765 | 9341.96 | 6028.535 | 2769.145 | 9176.68 |
| 3829.87 | 3988.305 | 6195.145 | 4211.35 | 2701.195 | 2657.725 |
| 5728.965 | 5815.7 | 7446.39 | 4354.85 | 3965.025 | 3542.095 |
| 6276.38 | 16792.635 | 7092.185 | 14944.545 | 3472.75 | 16541.58 |
| 4172.41 | 3266.835 | 5401.315 | 2697.59 | 7198.87 | 6813.37 |
| 7119.72 | 4734.4 | 7786.84 | 5028.775 | 4669.155 | 5400.605 |
| 6093.74 | 8304.8 | 11179.865 | 5642.76 | 2702.84 | 3219.39 |
| 3237.705 | 2061.98 | 3541.53 | 1930.69 | 1121.655 | 1095.625 |
| 38803.715 | 32004.57 | 37332.33 | 26437.735 | 21926.46 | 18605.91 |
| 3376.16 | 1939.21 | 2916.71 | 1189.795 | 1022.205 | 1593.325 |
| 4082.005 | 3957.145 | 4849.945 | 3138.535 | 1227.445 | 3639.465 |
| 2129.45 | 832.035 | 1677.16 | 461.235 | 654.825 | 1015.375 |
| 4103.855 | 3255.865 | 4194.135 | 2228.305 | 965.635 | 1179.295 |
| 7022.34 | 8468.155 | 6417.16 | 4989.4 | 3430.14 | 7897.355 |
| 3321.2 | 2548.06 | 2773.305 | 1631.145 | 1182.105 | 2413.33 |
| 7915.995 | 4218.805 | 8005.665 | 4073.855 | 3462.375 | 4780.93 |
| 1551.955 | 1209.865 | 1384.63 | 713.845 | 897.25 | 1941.37 |
| 1440.595 | 912.07 | 1357.55 | 566.54 | 549.48 | 1098.56 |
| 16430.37 | 14640.47 | 15924.765 | 10615.665 | 8498.015 | 9622.745 |
| 2581.37 | 532.48 | 4323.475 | 444.815 | 1802.645 | 1554.665 |
| 4995.4 | 6564.855 | 7663.89 | 7721.435 | 2645.275 | 5359.435 |
| 3360.67 | 5217.925 | 5061.2 | 4262.78 | 1722.515 | 4851.4 |
| 2075.27 | 1714.895 | 1696.535 | 1175.255 | 1909.175 | 2602.265 |
| 2807.44 | 5980.095 | 3110.525 | 3998.355 | 1268.215 | 3211.73 |
| 1645.83 | 917.225 | 1569.195 | 801.555 | 1064.605 | 1797.52 |
| 6632.915 | 6772.065 | 8987.995 | 5362.9 | 4152.205 | 7117.835 |
| 1946.93 | 1777.51 | 2597.16 | 1793.3 | 860.87 | 1390.535 |
| 2285.115 | 941.02 | 2373.325 | 676.97 | 880.48 | 1419.09 |
| 1861.36 | 1538.625 | 2237.21 | 1113.755 | 2386.835 | 1942.26 |
| 5461.58 | 8015.01 | 7962.22 | 9009.475 | 2215.405 | 4387.555 |
| 9810.315 | 9318.615 | 15293.165 | 7184.97 | 4834.465 | 3883.955 |
| 4142.37 | 4457.475 | 3815.11 | 2527.21 | 1768.885 | 2359.02 |
| 2648.03 | 3657.74 | 3876.66 | 3995.06 | 1664.575 | 3869.995 |
| 3519.87 | 4993.9 | 3656.605 | 3325.91 | 1313.975 | 4244.81 |
| 2195.015 | 1614.695 | 2055.28 | 1154.395 | 805.985 | 2101.195 |
| 8711.425 | 20472.21 | 14748.72 | 19734.92 | 8984.165 | 15204.445 |
| 1862.95 | 3365.855 | 1982.515 | 3837.725 | 1386.485 | 3064.65 |
| 8865.235 | 60468.53 | 8358.67 | 58067.745 | 13476.165 | 25023.29 |
| 1875.905 | 2584.39 | 2843.24 | 3481.685 | 1117.49 | 3175.01 |
| 4218.105 | 11513.8 | 6945.31 | 20725.45 | 2590.365 | 2174.52 |
| 2798.62 | 1516.665 | 2601.295 | 1136.315 | 1234.385 | 1865.25 |
| 5975.42 | 3057.395 | 6451.615 | 1841.065 | 2101.445 | 1645.4 |
| 1795.71 | 1198.81 | 1915.745 | 960.805 | 805.72 | 1075.18 |
| 5367.205 | 8405.4 | 6547.885 | 7373.495 | 2029.835 | 6714.13 |
| 4433.3 | 9745.615 | 6265.285 | 10524.195 | 4910.3 | 9971.195 |
| 1636.285 | 2724.855 | 2856.58 | 4443.245 | 1469.47 | 5004.25 |
| 2761.52 | 1518.285 | 2212.245 | 898.24 | 1361.28 | 2065.19 |
| 2470.15 | 2616.86 | 2552.875 | 1721.86 | 3376.135 | 2312.065 |
| 3715.225 | 1007.28 | 6610.615 | 782.37 | 2027.895 | 1872.38 |
| 6424.9 | 29897.1 | 7235.945 | 23528.355 | 4214.675 | 18458.71 |
| 1944.065 | 1803.385 | 1829.88 | 1421.275 | 570.14 | 1345.575 |
| 30318.855 | 25447.105 | 28098.58 | 13791.26 | 15221.425 | 17400.605 |
| 43837.395 | 46317.175 | 33279.11 | 24641.79 | 21664.195 | 47955.76 |
| 45156.825 | 46209.215 | 48681.125 | 38129.24 | 15857.825 | 26138.965 |
| 13512.11 | 12952.865 | 24228.06 | 15446.86 | 9104.735 | 12052.61 |
| 7233.835 | 7386.405 | 10611.365 | 8571.62 | 4640.325 | 5059.7 |
| 2252.845 | 1776.775 | 2743.17 | 1559.32 | 1746.005 | 2129.68 |
| 6232.38 | 4160.055 | 13000.67 | 5411.77 | 3493.845 | 3871.72 |
| 4309.39 | 2622.73 | 5827.245 | 2212.03 | 2491.495 | 2008.405 |
| 2739.255 | 4533.775 | 3713.645 | 5193.14 | 3982.21 | 5699.285 |
| 6307.16 | 9257.43 | 13374.635 | 10954.975 | 5441.695 | 11667.125 |
| 2870.74 | 25084.34 | 3549.05 | 29367.62 | 703.93 | 1338.635 |
| 2263.76 | 1263.57 | 2130.555 | 817.22 | 858.735 | 1164.11 |
| 1832.77 | 784.185 | 1487.23 | 467.66 | 636.375 | 868.235 |
| 2544.325 | 2890.16 | 2786.64 | 2745.725 | 1084.92 | 2273.695 |
| 1611.28 | 1726.175 | 1955.73 | 5943.055 | 824.625 | 3484.06 |
| 24142.96 | 9193.265 | 31951.88 | 6583.625 | 13059.64 | 7671.32 |
| 11072.16 | 51506.715 | 14292.945 | 46185.885 | 10124.42 | 44411.935 |
| 2674.72 | 633.755 | 2922.965 | 261.03 | 1322.645 | 2258.525 |
| 1594 | 804.405 | 1325.12 | 548.68 | 882.215 | 1591.12 |
| 56232.94 | 32145.855 | 58753.74 | 20462.255 | 45435.25 | 15190.265 |
| 3226.915 | 3862.28 | 4811.755 | 3441.28 | 4109.57 | 4660.58 |
| 2371.11 | 1660.52 | 2341.49 | 1225.815 | 701.6 | 2020.5 |
| 43315.82 | 58699.89 | 42801.98 | 55674.465 | 44355.69 | 40941.115 |
| 5214.2 | 3389.86 | 6792.995 | 2862.675 | 1970.855 | 1909.66 |
| 3344.59 | 1661.24 | 3438.355 | 1263.4 | 1433.245 | 2327.825 |
| 3199.235 | 3259.865 | 3999.81 | 2619.63 | 1167.305 | 5982.755 |
| 2394.21 | 1375.735 | 3250.925 | 1274.83 | 780.2 | 1216.53 |
| 2784.475 | 3431.04 | 2761.76 | 2333.045 | 2164.35 | 2060.87 |
| 12766.6 | 22470.75 | 11251.05 | 14002.74 | 16500.44 | 19215.345 |
| 2671.915 | 1429.835 | 3942.68 | 1409.405 | 902.835 | 2027.1 |
| 5676.9 | 7749.535 | 5439.61 | 5726.145 | 3350.85 | 7570.74 |
| 14299.02 | 14464.21 | 24819.865 | 14884.13 | 7075.025 | 7606.86 |
| 9668.765 | 50745.72 | 9744.19 | 34764.445 | 8190.415 | 19885.545 |
| 8377.21 | 6676.07 | 8061.83 | 3970.81 | 4095.63 | 4348.44 |
| 15826.63 | 9284.27 | 26103.785 | 7666.385 | 10330.3 | 6008.235 |
| 39184.425 | 32130.18 | 49237.09 | 29799.905 | 22240.965 | 29860.625 |
| 3991.815 | 6713.49 | 5611.3 | 6404.84 | 2643.39 | 6829.8 |
| 3184.965 | 3063.13 | 3220.55 | 4461.73 | 4009.25 | 8403.8 |
| 8714.435 | 10893.835 | 11598.16 | 9806.545 | 4632.94 | 2746.51 |
| 45028.745 | 42603.125 | 49054.695 | 43036.37 | 27685.87 | 25408.55 |
| 2686.31 | 3165.89 | 1918.755 | 2722.845 | 827.545 | 1595.495 |
| 2470.065 | 1268.585 | 1864.145 | 721.76 | 668.46 | 2046.935 |
| 7105.045 | 1975.355 | 12007.385 | 1723.415 | 4195.13 | 3835.305 |
| 4923.455 | 7527.59 | 7028.38 | 6704.82 | 4063.575 | 14160.36 |
| 3509.605 | 5112.58 | 5236.045 | 5015.675 | 1375.6 | 3964.015 |
| 2121.665 | 802.235 | 1631.45 | 489.08 | 4643.94 | 11886.16 |
| 10524.845 | 6031.4 | 18045.19 | 4612.785 | 6294.265 | 4633.54 |
| 4534.13 | 7168.02 | 5044.95 | 6239.845 | 6867.635 | 6948.925 |
| 7077.435 | 28177.855 | 7278.035 | 18025.505 | 3550.715 | 23372.47 |
| 5684.255 | 8375.295 | 7102.68 | 7075.1 | 5455.005 | 9633.99 |
| 2234.345 | 886.82 | 2438.08 | 629.36 | 963.62 | 1443.56 |
| 1959.21 | 610.725 | 1500.315 | 380.15 | 1941.31 | 12467.96 |
| 17188.865 | 16024.625 | 22802.135 | 12088.36 | 9689.11 | 11582.955 |
| 8618.41 | 9125.805 | 13858.705 | 9420.91 | 4853.49 | 2929.35 |
| 6158.155 | 7683.205 | 11203.19 | 12826.205 | 3794.715 | 8101.24 |
| 16440.415 | 37864.285 | 14619.81 | 26635.615 | 8511.455 | 23733.67 |
| 1632.45 | 511.585 | 1040.39 | 345.77 | 661.8 | 944.75 |
| 6646.235 | 55016.535 | 5488.075 | 40874.69 | 8068.81 | 21085.115 |
| 17641.77 | 12826.24 | 17413.3 | 7460.785 | 12562.41 | 10137.895 |
| 5275.285 | 15500.285 | 6191.025 | 19004.87 | 1418.84 | 5873.655 |
| 8262.21 | 15915.455 | 13267.58 | 16680.65 | 5956.745 | 20830.88 |
| 4579.005 | 9833.995 | 6379.13 | 10074.845 | 4707.05 | 14951.28 |
| 2132.9 | 1953.96 | 2976.015 | 1415.1 | 1018.365 | 1272.165 |
| 4418.04 | 8822.07 | 5956.59 | 6938.15 | 2186.855 | 4961.815 |
| 2115.02 | 1746.11 | 3588.565 | 2100.8 | 956.35 | 2240.115 |
| 3891.065 | 3545.16 | 4800.39 | 2556.45 | 1410.98 | 2437.27 |
| 3305.565 | 2706.675 | 5100.845 | 2512.01 | 2729.235 | 3043.81 |

What is claimed:

1. A micro array comprising a basal plate and a DNA fragment immobilized on the basal plate, the DNA fragment comprising an entire gene, a portion thereof, an EST thereof, or a portion of the EST, wherein the expression level of said DNA fragment is affected by a chemical substance having estrogen-like activity.

2. The microarray of claim 1, wherein the DNA fragment is selected from the group consisting of genes, gene portions, ESTs, or portions of the ESTs whose expression level changes in a breast cancer-derived MCF-7 cell in the presence of estrogen.

3. The microarray of claim 1, wherein the DNA fragment belongs to any one of groups (1), (2), and (3) below:
group (1): a gene, gene portion, EST, or portion of the EST whose expression level increases due to the presence of a chemical substance having estrogen-like activity, group (2): a gene, gene portion, EST, or portion of the EST whose expression level decreases due to the presence of a chemical substance having estrogen-like activity, and
group (3): a gene , gene portion, EST, or portion of the EST whose change in expression level arising due to the presence of a chemical substance having estrogen-like activity is smaller than that of groups (1) and (2) and whose expression level arising due to the presence of the chemical substance having estrogen-like activity is the same as or not less than that of a gene, gene portion, EST, or portion of the EST that is constitutively expressed regardless of the presence or absence of the chemical substance having estrogen-like activity.

4. The microarray of claim 3, wherein the microarray further comprises more than one DNA fragment immobilized on the basal plate, wherein said additional DNA fragment(s) are selected from the group consisting of genes, gene portions, ESTs, or portions of the ESTs belonging to the group (1), genes, gene portions, ESTs, or portions of the ESTs belonging to the group (2), and genes, gene portions, ESTs, or portions of the ESTs belonging to the group (3).

5. The microarray of claim 1, further comprising an additional DNA fragment immobilized on the basal plate, wherein the additional DNA fragment comprises a gene, gene portion, EST, or portion of the EST that is constitutively expressed regardless of the presence or absence of a chemical substance having estrogen-like activity.

6. The microarray of claim 1, wherein the DNA fragment belongs to any one of groups (4), (5), (6), (7), and (8) below:
group (4): a gene, gene portion, EST, or portion of the EST related to cancer,
group (5): a gene, gene portion, EST, or portion of the EST involved in cell proliferation and/or division,
group (6): a gene, gene portion, EST, or portion of the EST encoding a polypeptide functioning as a receptor,
group (7): a gene, gene portion, EST, or portion of the EST involved in transcription reaction, and
group (8): a gene, gene portion, EST, or portion of the EST involved in cell structure.

7. The microarray of claim 6, where in the microarray further comprises more than one DNA fragment immobilized on the base plate, wherein said additional DNA fragment(s) are selected from the group consisting of genes, gene portions, ESTs, or portions of the ESTs belonging to the group (4), genes, gene portions, ESTs, or portions of the ESTs belonging to the group (5), genes, gene portions, ESTs, or portions of the ESTs belonging to the group (6), genes, gene portions, ESTs, or portions of the ESTs belonging to the group (7), and genes, gene portions, ESTs, or portions of the ESTs belonging to the group (8).

8. The microarray of claim 1, wherein the DNA fragment belongs to any one of groups (9), (10), and (11) below:
group (9): a gene, gene portion, EST, or portion of the EST functioning in a tissue on which estrogen acts,
group (10): a gene, gene portion, EST, or portion of the EST characteristic of an epithelial cell, and
group (11): a gene, gene portion, EST, or portion of the EST functioning in a nerve cell.

9. The microarray of claim 8, wherein the microarray further comprises more than one DNA fragment immobilized on the base plate, wherein said additional DNA fragment(s) are selected from the group consisting of genes, gene portions, ESTs, or portions of the ESTs belonging to the group (9), genes, gene portions, ESTs, or portions of the ESTs belonging to the group (10), and genes, gene portions, ESTs, or portions of the ESTs belonging to the group (11).

10. The microarray of claim 1, wherein the DNA fragment belongs to group (12) or (13) below:
group (12): a gene, gene portion, EST, or portion of the EST whose expression level increases due to the presence of estrogen in a cell resistant to an antagonist of estrogen, and
group (13): a gene, gene portion, EST, or portion of the EST whose expression level decreases due to the presence of estrogen in a cell resistant to an antagonist of estrogen.

11. The microarray of claim 10, wherein the microarray further comprises more than one DNA fragment immobilized on the base plate, wherein said additional DNA fragment(s) are selected from the group consisting of genes, gene portions, ESTs, or portions of the ESTs belonging to the group (12), and genes, gene portions, ESTs, or portions of the ESTs belonging to the group (13).

12. A method for evaluating the estrogen-like activity of a compound, the method comprising utilizing the microarray of claim 1.

13. A method for predicting the effect on a cell of a compound having estrogen-like activity, the method comprising the steps of:
(a) preparing a nucleic acid sample from a cell to be evaluated,
(b) contacting the nucleic acid sample with the microarray of any one of claims 1 to 11,
(c) detecting a nucleic acid hybridizing with the microarray, and
(d) comparing a result detected in step(c) with a result detected using a nucleic acid sample prepared from a control cell.

14. The method of claim 13, wherein the control cell is an MCF-7 cell treated with 17β-estradiol.
